(19) United States
(12) Patent Application Publication
(10) Pub. No.: US 2025/0339542 A1
(43) Pub. Date: Nov. 6, 2025

(54) CONVERSION OF A BIOLOGICALLY SILENT MIRNA BINDING SMALL MOLECULE TO AN MIRNA DEGRADER

(71) Applicant: University of Florida Research Foundation, Incorporated, Gainesville, FL (US)

(72) Inventor: Matthew D. Disney, Gainesville, FL (US)

(73) Assignee: University of Florida Research Foundation, Incorporated, Gainesville, FL (US)

(21) Appl. No.: 18/023,456

(22) PCT Filed: Aug. 27, 2021
(86) PCT No.: PCT/US2021/047936

§ 371 Date: Feb. 27, 2023

Related U.S. Application Data
(60) Provisional application No. 62/706,615, filed on Aug. 28, 2020.

(51) Int. Cl. A61K 47/55; C07D 233/64; C07D 409/12; C07D 495/04

(52) U.S. Cl. CPC A61K 47/55; C07D 233/64; C07D 409/12; C07D 495/04

(57) ABSTRACT

Biologically silent small molecules that bind to RNA motifs but do not modify or negate the bioactivity of the RNA motif can be conjugated with an RNAase activator thereby degrading the RNA motif and negating its bioactivity.

### CROSS-REFERENCE TO RELATED APPLICATIONS

The subject patent application is a national stage filing under 35 U.S.C. 371 of International Patent Application Serial No. PCT/US2021/047936, filed Aug. 27, 2021, entitled “CONVERSION OF A BIOLOGICALLY SILENT MIRNA BINDING SMALL MOLECULE TO AN MIRNA DEGRADER”, which claims the benefit of priority to U.S. Application No. 62/706,615, filed Aug. 28, 2020. The full disclosure of the priority applications are incorporated herein by reference in their entirety and for all purposes.

### STATEMENT OF GOVERNMENT SUPPORT

This invention was made with government support under Contract Nos. CA249180 and GM097455 awarded by the National Institutes of Health. The government has certain rights in this invention.

### BACKGROUND

RNA structures play regulatory roles throughout all kingdoms of life and all cells and RNAs play a pervasive role in disease making it an attractive drug target.⁴⁰ One challenge is to identify small molecules that bind to (or drug) these structures to affect function and affect disease phenotypes. Two modalities exist for the drugging of RNA, antisense oligonucleotides (ASOs) and small molecules. The former, recognizes its target by base pairing, triggering degradation of the transcript by host machinery. Therefore, it was once thought that ASOs can target any transcript selectively. However, limitations have arisen with this modality such as the influence of RNA structure on the efficiency of ASO binding, limiting their efficacy.² Recent studies have demonstrated that RNA is in fact rich in structure⁴¹, and this inherently limits the targeting potential of ASOs to be confined to regions that are unstructured or dynamic. To fully utilize the drug potential of RNA it was proposed that small molecules could be a used to drug RNA broadly, however this was thought impossible due to RNAs flexibility.⁵

This notion has since been challenged as much effort has focused on identifying bioactive RNA structure binding molecules. At the forefront of this is a methodology called 2DCS⁹, that leverages high throughput screening and next generation sequencing to rapidly identify and annotate the RNA fold binding preferences of small molecules and collates them into a database of RNA fold small molecule interactions termed Inforna⁹. Since Inforna's development, a litany of molecules has been shown potently and selectively to target RNA structure and modulate disease phenotypes, however many challenges remain.^{23,29,33,42-44} These include, understanding the relationship between small molecule chemical structure and RNA fold binding, and expanding the RNA fold targeting scope beyond functional sites.

### SUMMARY

The present invention is directed to methods and compositions involving binding or cleaving (and hence drugging) micro-RNAs (miRNAs) with small molecules and enabling the cleavage, decomposition and/or concentration reduction of the miRNAs. As explained above, miRNAs play a key role in RNA silencing and in up- and down- regulation of RNA expression. The miRNA's typically have three-dimensional structures that are amenable to binding with small molecules. In the past, the small molecule-miRNA binding to functional Dicer and Drosha process sites in their precursors has been found to inhibit the miRNA's biogenesis and hence reduce levels of mature miRNA, the aberrant expression of which can cause disease. These small molecules can then be converted into ribonuclease (RNase) recruiters that also cleave miRNA precursors. The present invention, in contrast, is directed to small molecule-mRNA binding that by itself is biologically silent. That is, the binding process does not elicit inhibition of biogenesis and hence does not change the levels of pri-miRNA, pre-miRNA, or the mature, active miRNA. To achieve bioactivity, i.e., the reduction of the active, mature miRNA, the biologically silent binding small molecules have been conjugated with a moiety that activates an RNase to degrade and/or cleave the target miRNA precursor, thereby reducing the concentration levels of the mature miRNA.

The methods according to the present invention are directed to contact of a mixture comprising one or more miRNA and RNase with a compound of Formula I:

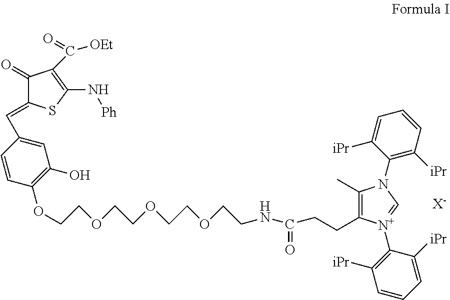

Preferably the gegenions may be chloride, sulfate, nitrate, phosphate, acetate, trifluoroacetate, mesylate or benzoate. The miRNAs comprise pri-miR-155, pre-miR-155, miR-155 and any combination thereof.

The methods according to the present invention are also directed to conversion of a biologically silent (biologically inactive) miRNA binding moiety into a biologically active compound that will cleave pri- and pre miRNAs to interrupt and/or otherwise ameliorate the biogenesis of mature miRNAs. The conversion comprises covalently linking the biologically inactive miRNA binding moiety to an RNase recruiting moiety through a polyoxyethylene amine linker. To accomplish the linkage, precursors of the biologically inactive miRNA binding moiety may be converted to a carboxylic acid derivative having approximately similar binding constant with the miRNA target. The carboxylic acid derivative is amidated with a polyoxyethylene amine carrying at its opposite terminus the RNase recruiting moiety. The resulting biologically active compound has Formula V wherein Group A is the amidated version of the carboxyl derivative of the biologically inactive miRNA binding moiety and Group B is the RNase recruiting moiety.

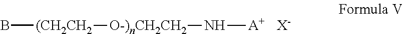

A preferred embodiment of the biologically active compound comprises Formula V in which Group A of Formula V is Moiety A and Group B of Formula V is Moiety B:

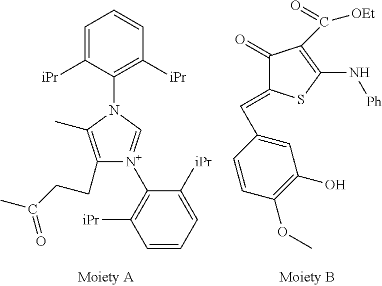 [SMILES: CC(=O)CCc1c(C)n(-c2c(C(C)C)cccc2C(C)C)c[n+]1-c1c(C(C)C)cccc1C(C)C ; CCOC(=O)C1=C(Nc2ccccc2)S/C(=C\c2ccc(OC)c(O)c2)C1=O]

X comprises a gegenion and n is an integer of 3 to 5, preferably 3. The gegenion is an organic or inorganic anion forming a salt with Formula V.

Cleavage of miRNAs with a compound of Formula V may be accomplished by contacting the compound of Formula V with a mixture of at least an RNase and an miRNA to which Group A has shown strong binding affinity. The miRNAs suitable for this embodiment include pri-miRNAs and pre-miRNAs.

The present invention is further directed to compounds of Formulas I (depicted above), II, III, IV and V. Formulas II and III are biologically silent miRNA binding compounds, that is, they have no effect on the biogenesis of miR-155 or the levels of pri-miR-155, pre-miR-155 or miR-155. Formula III is the simple alkyl amide form of the carboxylic acid Formula II. Formula IV is similar to Formula I except that the recruiting moiety is bound to the PEG moiety by its meta oxygen which renders the recruiting moiety inactive.

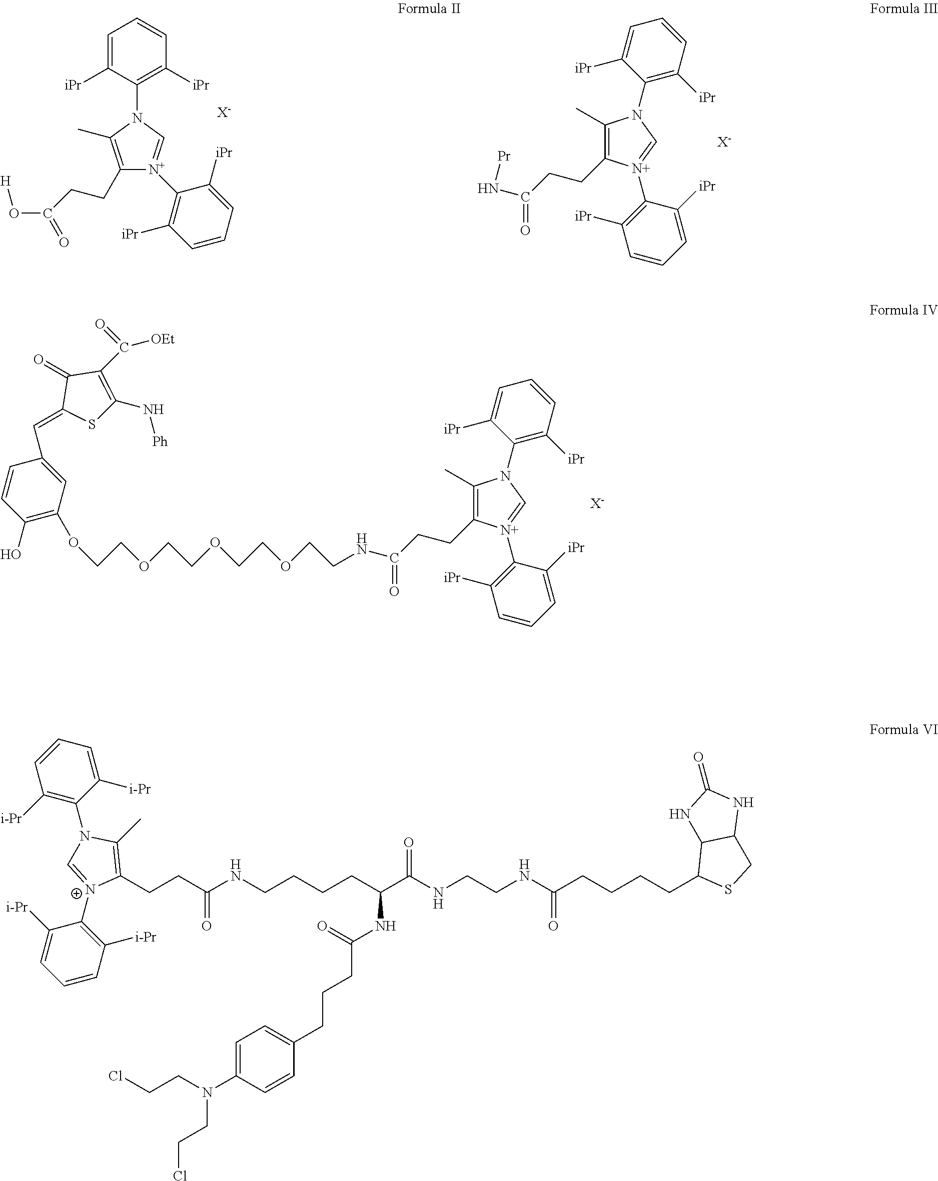

The Detailed Description describes the relationship between the biologically silent Formulas II and III and their precursor Compound C2(1). Compound C2(1) has the formula

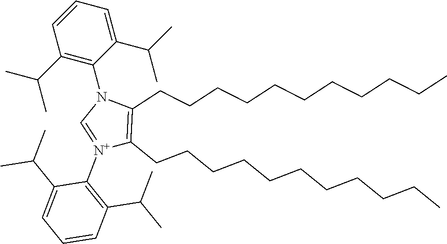 [SMILES: CCCCCCCCCCCc1c(CCCCCCCCCCC)[n+](-c2c(C(C)C)cccc2C(C)C)cn1-c1c(C(C)C)cccc1C(C)C]

According to the invention, Compound C2(1) displays significant, selective binding with miR-155 precursors but is biologically silent, i.e., is inactive. It does not inhibit miRNA biogenesis. To overcome this difficulty and expand the range of small molecules capable of intersecting and modifying the biological activities of miRNAs, Compound C2(1) was repeatedly synthetically modified to eventually produce experimentally an Azolium compound that could be synthetically combined with an RNase recruiting moiety and at the same time exhibit the significant selective binding with the miRNA target similar to the binding of Compound C2(1).

The compound of Formula IV is similar in structure to the compound of Formula I except that the ether bond of Moiety B to the PEG chain of Formula IV is through the meta oxygen of Moiety B instead of the para oxygen as in Formula I. This meta arrangement delivers an inactive RNase L-recruiting moiety. Formula IV serves as a control agent for assessing the specificity and bioactivity of Formula I.

Formula I may be combined with an in cellulis mixture of one or more of the miRNA's and RNase L to demonstrate its bioactivity against the miRNA's. Preferably, the mixture constitutes a constituent of cultured cells such as breast cancer cells MDA-MD-231 or natal umbilical cells, MUVEC cells. In the context of in vitro activity, the compound of Formula I exhibits an IC₅₀ against pre-miR-155 at no more than about 0.1 micromolar. In MDA-MD-231 and/or HUVEC cells, the compound of Formula I degrades pre-miR-155 by at least approximately 60% at a concentration of 0.1 micromolar. The compound of Formula I also exhibits a dose related response against miR-155 in the context of MDA-MD-23land/or HUVEC cells at concentrations ranging from 1 picomolar to 100 nanomolar. Dose related response ranges from 40% to 80% inhibition as the concentration of Formula I increases.

MDA-MD-231 may be transfected into an animal host such as a rat or mouse and the cells may be allowed to multiply to form a tumor. Administration of pharmaceutical composition of the compound of Formula I given as an iv or ip dose to the host may establish suppression of the tumor and remission of the cells.

Treatment with embodiments of the invention may also be directed to human diseases in which miR-155 is overexpressed, including cancer, neuroinflammation and neurodegeneration among others. Pharmaceutical compositions of Formula I in a pharmaceutically acceptable carrier serve as appropriate administration embodiments for such treatments. An example of such treatment involves MDA-MD-231 cells which may be present in a human patient having breast cancer. Treatment with a compound of Formula I given as an iv or ip dose as described in the following sections on Administration may ameliorate the breast cancer. Preferably, appropriate administration of a pharmaceutical composition of the compound of Formula I may be given as an iv or ip dose to ameliorate the cancer.

### BRIEF DESCRIPTION OF FIGURES

FIGS. 1A-1D disclose novel chemotypes that illicit identify novel binding interactions for sequence- based design targeting of RNA.

FIGS. 1A-1B disclose a schematic of the TO-Pro-1 fluorescent indicator displacement assay shows that TO-Pro-1 binds to the randomized region of the 3×3 ILL and exhibits enhanced fluorescence. Displacement of TO-Pro-1 by members of the 15,000 member COMAS library identified 330 hits, comprised of 20 novel scaffolds.

FIG. 1C discloses that the selectivity for binding RNA was studied by two-dimensional combinatorial screening (2DCS), probing 61,440,000 interactions, identifying four novel chemotypes including bipyrrolo pyrrolium salts (red), azolium salts (blue), chromones (purple) and 3-phenylfuro[3,2-b]pyridine-5-amines (black). These studies yielded 98 previously undiscovered RNA-small molecule interactions.

FIG. 1D discloses that motifs identified by the 2DCS studies herein were cross referenced to all human miRNAs, identifying 1,075 targetable miRNAs. Of these miRs, 750 miRs were only targetable in at a functionally silent site However, 90% (657) of these miRNAs also contained a potential Ribonuclease targeting Chimera (RIBOTAC) substrate. This strategy opens the door to targeting any RNA, regardless if the small molecule binding site is functional, with an RNA degrader to modulate its function.

FIGS. 2A-2E disclose that RIBOTACs activate ligandable sites in RNAs that contain motifs sensitive to RNase L.

FIGS. 2A-2B show how precursor microRNA 155 (pre-miR-155) was identified to contain both a functionally silent small molecule binding site and a RIBOTAC sensitive motif. Treatment with a RIBOTAC recruiter can therefore activate RNase L and trigger degradation of pre-miR-155, de-repressing SOCS1 and inhibiting migration.

FIG. 2C shows that Compound 2 is the modified binder to pre-miR-155 which binds with the same affinity as 1 to the 5′GAU/3′C_A bulge. Conversion to a RIBOTAC, with the synthetic recruiter C13³⁰ yielded compound 3.

FIG. 2D shows that simple binding to a non-functional site had no effect on pre-miR-155 levels (n=3).

FIG. 2E shows that treatment with a RIBOTAC (3) resulted in degradation of pre-miR-155, decreasing its expression levels (n=3). All errors are reported as the mean±S.E.M. of the measured biologically independent replicates. All p-values calculated by two-tailed Students T-test, **, p<0.01.

FIGS. 3A-3C show proteome wide upregulation of miR-155 associated targets inhibits migration in MDA-MB-231 cells.

FIG. 3A shows a global proteomics analysis of MDA-MB-231 cells treated with 3 identified 3,158 proteins, of which 98 were associated with miR-155. Comparison of treated and untreated samples showed that compound 3 significantly upregulated their expression, as indicated by a Kolmogorov-Smirnov analysis of their levels relative to all proteins (n=3).

FIG. 3B shows a western blot analysis of SOCS1 shows that 3 de-represses the target by 50% (n=3).

FIG. 3C shows a Migration analysis of MDA-MB-231 cells which is inhibited by compound 3 and is comparable to and LNA targeting miR-155 (n=3). All errors are reported as the mean±S.E.M. of the measured biologically independent replicates. All p-values are calculated using a two-tailed students t-test. *, p<0.05; **, p<0.01; ***, p<0.001.

FIGS. 4A-4E show TO-Pro-1 screening validation and results for COMAS collection.

FIG. 4A shows optimization of the signal to noise ratio shows that 200 nM of the 3×3 ILL And 200 nM of TO-Pro-1 are optimal for screening.

FIG. 4B shows, using Hoechest 33258 as a positive control, that using a 5-fold signal to noise ratio, Hoeches 33258 is able to achieve higher displacement at 10 μM with 200 nM of RNA compared to 100 nM of RNA.

FIG. 4C shows a Z-factor analysis of the screening conditions shows that using a concentration of 10 μM enables better discrimination between the positive control (Hoechest 33258) and the negative control (DMSO).

FIGS. 4D-4E show the 330 hits identified to dose responsively bind RNA, they classified into 20 unique scaffolds, of which 14 are novel, and 6 have been previously reported.

FIGS. 5A and 5B show that new hits from 2DCS are chemically dissimilar to known RNA binding matter.

FIG. 5A shows Tanimoto analysis of compounds C1-C20 compared to all 404 compounds within Inforna show a mean Tanimoto coefficient between 0.3 and 0.4 indicating that they are not similar.

FIG. 5B shows a comparison of C1 to C20 to the 104 compounds in R-BIND also show mean Tanimoto scores in the same range of 0.3 to 0.4 indicating low similarity. These indicate that the compounds are indeed novel compared to known RNA binders. Tanimoto scores generated using instant JChem (ChemAxon).

FIGS. 6A-6C graphically illustrate the unique chemical patterns indicate chemically similar compounds within the novel azolium scaffold. Compounds C7, C8, C9, and C10 have a Tanimoto score of 0.7-1.0, and cluster together. Compounds C12-C15 are chemically identical as they share the cholesteryl azolium core differing only by alkyl chain substitute. However, when compared to all other hits their Tanimoto score ranges from 0.29 -0.32 indicating they are unique among all the hits obtained. Compound C2 and C11 exhibit high similarity (Tanimoto: 0.82) although they appear very different structurally. This could be due to similar spatial orientation of the compounds which both have alkyl substituted benzenes on their azolium cores. Compounds C4, C5, C6 and C20 are structurally unique compared to all other hits.

FIG. 7 provides a LOGOS analysis of C1 to C6 showing enriched and discriminated sequences. FIG. 7 includes tables of the SEQ ID NO's for these RNA motifs.

FIG. 7 illustrates the Enriched nucleotides for the top 0.5% of sequences in the 3×3 ILL. These show a high preference for adenine and cytosine in the motifs they bind. Note: these motifs all have a fitness score >85% and are near exclusively 3×3 internal loops.

FIG. 7 illustrates nucleotides and RNA motifs that are preferentially not bound by each compound. These molecules show low propensity to bind motifs with GC closing pairs. They also almost exclusively do not bind 2×2 internal loops a feature not previously observed. Note: this analysis is based on the top 0.5% of enriched/discriminated sequences for each molecule.

FIG. 8 illustrates LOGOS of C7 to C13 for enriched and discriminated sequences.

FIG. 8 includes tables of the SEQ ID NO's for these RNA motifs.

FIG. 8 shows that globally compounds C7 to C13 prefer motifs rich in C and A, with nucleotide six of the randomized region being almost exclusively adenine. Similar to C1 -C6, 3×3 IL's are the predominantly bound motif (75%) with single nucleotide bulges and 1×1 and 2×2 internal loops occupying 14% of the remaining 25%. Interestingly, compounds C7 and C11 bind a randomized region that forms stable alternating (AU)(UA) base pairs as their highest fitness interaction (pink box). This is the first ever demonstration of selective base pair binders being identified in a target agnostic fashion.

FIG. 8 shows that discriminated motifs are predominantly bulges and lxi or 2×2 internal loops, 82% of motifs. These sequences are rich in C or U at nucleotide positions four, five, and six with position six being primarily U. Note: this analysis is based on the top 0.5% of enriched/discriminated sequences for each molecule.

FIG. 9 discloses LOGOS of C14-C20 for enriched and discriminated sequences.

FIG. 9 includes tables of the SEQ ID NO's for these RNA motifs.

FIG. 9 shows that enriched sequences for all compounds are rich in C and A with position one and position six being G and A respectively. The most variable nucleotide is position three which can be either C, G or A. Note that 93% of motifs are 3×3 IL's with the only closing pair being AU pairs.

FIG. 9 shows motif that are discriminated against are primarily lxi and 2×2 IL's, similar to C7-C13 described above. There is also a higher incidence of GC closing pairs (30%) compared to no GC closing pairs for enriched motifs. These motifs are also rich in U at positions four and six, which would suggest formation of either GU or AU pairs for smaller motifs like bulges and 1×1 internal loops which are observed. Note: this analysis is based on the top 0.5% of enriched/discriminated sequences for each molecule.

FIGS. 10A-10C show that compound C2 (1) binds to precursor miR-155 (pre-miR-155) at a non-functional site near an RNase L sensitive motif.

FIGS. 10A and 10B together show that cross referencing the HiT-StARTS analysis of compounds C1-C20 with the microRNAs that contain ligandable non-functional sites and RIBOTAC substrates and the human miRNA disease database (HMDD v3.0)² identified that C2 (1) binds a non-functional site in pre-miR-155 (SEQ ID NO:11) (5′GAU/3′C_A) and pre-miR-410 (SEQ ID NO:114) (5′CCU/3′G_A). Due to miR-155's broad role in multiple disease indications like cancer^{3, 4} and neuroinflammation and neurodegeneration^{5, 6}, there is a focus on pre-miR-155 for further study. Pre-miR-410 is also implicated in hepatic cancer however its disease scope is limited.⁷

FIG. 10C illustrates the binding affinity of compound 1 to miR-155's binding site was measured by microscale thermophoresis. This shows that 1 binds to the A bulge with a Ka of 490±122 nM. Since conjugation of the RIBOTAC recruiter requires a linker, the n-undecyl chains were replaced with a propionic space and amidated mimic a conjugated giving compound 2. Its affinity was also measured and found to be similar to that of 1 with a Ka of 552±120 nM. Neither molecule showed significant binding to the base paired control or the C bulge. Sequences of the different sites are shown (SEQ ID NOs:127-130, respectively).

FIGS. 11A and 11B show that Compound 3 cleaves pre-miR-155 in vitro and its cleavage is competed off by 1.

FIG. 11A shows treatment of Pre-miR-155 with compound 3 and recombinant RNase L. Dose dependent cleavage of pre-miR-155 is observed with and IC₅₀ of −100 nM (n=3).

FIG. 11B shows that parent compound 1 and compound 3 bind to the same site, competitive RNase L cleave was done. This shows that compound 1 can dose dependently reduce cleavage of pre-miR-155 by 3 (IC_{50=1.9±0.5} RM), indicating the compounds compete for binding to the same site (n=3). All errors are reported as the mean±S.E.M. of the measured experimentally independent replicates. All p-values are calculated using a one-way ANOVA with multiple comparisons where *, p<0.05; **, p<0.01; ***, p<0.001.

FIGS. 12A and 12B show that mutation of the RNase L cleavage site and small molecule binding site in pre-miR-155 ablates compound 3 activity.

FIG. 12A shows that in vitro cleavage of mutant pre-miR-155 is ablated upon mutation of the cleavage site. This indicates that this structure is sensitive to RNase L cleavage and is required for compound 3's activity (n=3). FIG. 12B shows that mutation of the small molecule binding site also ablates RNase L cleavage with compound 3 indicating that it is necessary for 3 to engage the RNA and recruit RNase L to cleave it (n=3).

FIG. 13 shows that control RNase L recruiter which lacks the RNA binding module, compound 4, is unable to cleave pre-miR-155 in vitro. The RNase L recruiter lacking the RNA binding module, compound 4, is inactive at recruiting RNase L to cleave pre-miR-155 (n=3). This indicates that engagement of the small molecule with the RNA is required for cleavage.

FIGS. 14A-14E show that Compound 3 selectively cleaves miR-155 and its effect is competed off by compound 1 in MDA-MB-231 cells.

FIG. 14A provides an analysis of pre-miR-155 levels shows that compound 3 cleaves pre-miR-155, decreasing its levels. When co-treated with 1, the effect is ablated dose dependently, indicating that 3 and 1 bind to pre-iR-155 at the same site (n=3).

FIG. 14B shows graphs various levels of RT-qPCR of pri-miR-155 and shows that 3 also engages the primary transcript of miR-155 and degrades it, decreasing its levels. When co-treated with 1 this effect is ablated dose dependently (n=3).

FIG. 14C shows that Compound 3 decreases levels of mature miR-155 in MDA-MB-231 cells. When co-treated with parent compound 1, the effect of 3 is ablated dose dependently (n=4).

FIG. 14D shows that cleavage is dependent on compound 3, MDA-MB-231 cells were treated with 3 for 48 h and the media was then removed and RNA harvested over time. Rescue of pre-miR-155 levels after compound removal shows that degradation is dependent on compound 3 with t_{1/2}=19.3 h, which is in accordance with reported half-lives of miR-155 that range from 12.4-28 h.

FIG. 14E shows MicroRNA profiling of 3 compared to 373 expressed miRNAs shows that miR-155 is the most significantly downregulated miRNA (n=3). All errors are reported as the mean±S.E.M. of the measured biologically independent replicates. All p values for panels A, B, and C were calculated using a one-way ANOVA adjusted for multiple comparisons. P values for panels D and E were calculated by a Students T-test assuming equal variance. *, p<0.05; **, p<0.01; ***, p<0.001.

FIGS. 15A and 15B show that Compound 3 does not affect isoforms of miR-155 in MDA-MB-231 cells.

FIG. 15A provides RNA isoforms of miR-155 identified from Targetscan and a corresponding table of the isoform SEQ ID NO'S.

FIG. 15B shows that Compound 3 has no effect on all RNA isoforms of miR-155 that contain the small molecule binding site over 48 h (n=3). All errors are reported as the mean±S.E.M. of the measured biologically independent replicates.

FIGS. 16A-16F show that simple binding compounds 1, 2, and control compound 4 have no effect on the mature precursor and primary transcripts of miR-155 in MDA-MB-231 cells.

FIGS. 16A and 16B together show that Compound 1 is unable to inhibit miR-155 biogenesis over 48 h (panel B: n=3; panel C: n=3).

FIGS. 16C and 16D together show that Compound 2, the amidated RNA binder lacking the RNase L recruiter has no effect on miR-155 biogenesis over 48 h (n=3). All errors are reported as the mean±S.E.M. of the measured biologically independent replicates.

FIGS. 16E and 16F together show that Compound 4, the RNase L recruiter lacking the RNA binding module (2) does not affect miR-155 biogenesis over 48 h (panel A: n=3; panel B: n=3).

FIGS. 17A-17C show that Control compound 5 does not affect the mature, precursor or primary transcripts of miR-155 in MDA-MB-231 cells.

FIG. 17A illustrates the structure of the inactive RIBOTAC recruiter conjugated onto compound 2 to afford control compound 5.

FIGS. 17B and 17C together show that Compound 5, the inactive structural isoform of the RNase L recruiter does not inhibit miR-155 biogenesis over 48 h (panel E: n=3; panel F: n=3). All errors are reported as the mean±S.E.M. of the measured biologically independent replicates.

FIGS. 18A-18C show that Compound 6 directly engages pre-miR-155 and is competed off by compound 1 as shown by Chemical crosslinking and isolation by pulldown (Chem-CLIP) and competitive Chem-CLIP (C-Chem-CLIP) in MDA-MB-231 cells.

FIG. 18A shows that the Chem-clip probe is obtained by conjugating compound 2 with a chlorambucil (CA) reactive module (blue triangle) that crosslinks to the RNA, and a biotin pulldown module (gold circle) to enrich the RNA samples for cross-linked RNA, affording compound 6. The control compound is the reactive and pulldown handles which lack the RNA binding module, yielding compound 7.

FIG. 18B shows that Chem-CLIP functions by using the interaction of the small molecule to bring the reactive module in close proximity with the RNA to react. Once reacted, the direct targets of the molecule can be enriched by pulldown and assessed by RT-qPCR.

FIG. 18C demonstrates that with respect to RT-qPCR of pulldown fractions, compound 7, which is the reactive probe that lacks the RNA binding modules, is unable to pull down pre-miR-155, however, treatment with compound 6 at 100 nM results in a 7-fold enrichment of pre-miR-155. When co-treated with compounds 6 and 1, compound 1 dose dependently depletes enrichment of pre-miR155, indicating that 6 and 1 compete for the same binding site (n=3). All errors are reported as the mean±S.E.M. of the measured biologically independent replicates. All p values are calculated using a one-way ANOVA adjusted for multiple comparisons **, p<0.01.

FIGS. 19A-19D show that Compound 3 directly recruits RNase L to pre-miR-155 and modulates its expression levels in an RNase L dependent fashion.

FIGS. 19A-19C together show measurement of mature, pre- and pri-miR-155 levels in MDA-MB-231 cells transfected with an siRNA that knocked down RNase L by 85%. This resulted in ablation of degradation on the precursor and primary transcripts resulting in no effect being observed on all three transcripts (n=3).

FIG. 19D shows confirmation that compound 3 in fact recruits RNase L to pre-miR-155, RNase L was immunoprecipitated from MDA-MB-231 cells. Analysis of pulldown fractions by RT-qPCR showed that cells treated with 3 had 2-fold enrichment of pre-miR-155 compared to untreated, while cells treated with the inactive recruiter 5, showed no enrichment relative to untreated. This confirms that 3's mode of action is dependent on RNase L (n=3). All errors are reported as the mean±S.E.M. of the measured biologically independent replicates. All p values were calculated by a Students T-test assuming equal variance. *, p<0.05.

FIGS. 20A-20C show that the migratory phenotype of miR-155 is binding site dependent in healthy MCF-10a cells expressing wild type or mutant pre-miR-155.

FIG. 20A shows that mock transfection of MCF-10a cells has no effect on their migratory capacity, and compound 3 is inactive, as expected (n=3).

FIG. 20B shows that overexpression of wild type miR-155 triggers migration in MCF-10a. When treated with compound 3, this migratory activity is reduced by 50% at 1×10-7 M (n=3).

FIG. 20C shows that over expression of mutant miR-155 also causes migration of the MCF-10a cells, however it is unaffected by compound 3 since the molecule cannot bind and degrade the mutant precursor transcript (n=3). All errors are reported as the mean±S.E.M. of the measured biologically independent replicates. All p-values are calculated using a two-tailed students t-test assuming equal variance. ***, p<0.001.

FIGS. 21A-21I show that Compound 3 decreases levels of pre-miR-155 and decreases angiogenesis in HUVECs.

FIGS. 21A and 21B together show that treatment with the parent compound 1 has no effect on miR-155 or pre-miR-155 levels in HUVECs (panel A:n=3; panel B: n=4).

FIGS. 21C and 21D together show that Compound 3 dose dependently decreases miR-155 and pre-miR-155 levels in HUVECs with 50% inhibition at 1×10⁻⁹ M (panel C:n=3; panel D: n=4).

FIGS. 21E and 21F together show that treatment with the control compound 5 has no effect on miR-155 and pre-miR-155 levels confirming that the binding module is required for RNase L activity.

FIG. 21G shows that Compound 3 degrades pre-miR-155 which can be competed off using compound 1 in a dose dependent manner (n=4).

FIG. 21H shows that Pre-miR-155's downstream target, Von-Hippel Lindau (VHL) is de-repressed by 50% at 10 nM of compound treatment (n=3).

FIG. 21I shows that Compound 3 modestly inhibits the angiogenic capacity of HUVECs by 20% (n=2). All errors are reported as the mean±S.E.M. of the measured biologically independent replicates. All p values are calculated using a two-tailed Students t-test except for panel A which uses a one-way ANOVA with multiple comparisons. *, p <0.05; **, p<0.01; ***, p<0.001.

FIG. 22 shows that Compound 3 does not affect isoforms of miR-155 that contain the same binding site in HUVEC cells. (n=3). All errors are reported as the mean±S.E.M. of the measured biologically independent replicates.

### DEFINITIONS

Unless defined otherwise, all technical and scientific terms used herein have the same meaning as commonly understood by a person of ordinary skill in the art.

The term “about” as used herein, when referring to a numerical value or range, allows for a degree of variability in the value or range, for example, within 10%, or within 5% of a stated value or of a stated limit of a range.

All percent compositions are given as weight-percentages, unless otherwise stated.

All average molecular weights of polymers are weight-average molecular weights, unless otherwise specified.

The term “may” in the context of this application means “is permitted to” or “is able to” and is a synonym for the term “can.” The term “may” as used herein does not mean possibility or chance.

It is also to be understood that as used herein and in the appended claims, the singular forms “a,” “an,” and “the” include plural reference unless the context clearly dictates otherwise.

The term “X and/or Y” means “X” or “Y” or both “X” and “Y”.

The letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and the right is reserved to revise the application or claims to refer specifically to any individual member or any subgroup of members of the Markush group.

The expression “effective amount”, when used to describe therapy to an individual suffering from a disorder, refers to the amount of a drug, pharmaceutical agent or compound of the invention that will elicit the biological or medical response of a cell, tissue, system, animal or human that is being sought, for instance, by a researcher or clinician. Such responses include but are not limited to amelioration, inhibition or other action on a disorder, malcondition, disease, infection or other issue with or in the individual's tissues wherein the disorder, malcondition, disease and the like is active, wherein such inhibition or other action occurs to an extent sufficient to produce a beneficial therapeutic effect. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

“Substantially” as the term is used herein means completely or almost completely; for example, a composition that is “substantially free” of a component either has none of the component or contains such a trace amount that any relevant functional property of the composition is unaffected by the presence of the trace amount, or a compound is “substantially pure” is there are only negligible traces of impurities present.

“Treating” or “treatment” within the meaning herein refers to an alleviation of symptoms associated with a disorder or disease, or inhibition of further progression or worsening of those symptoms, or prevention or prophylaxis of the disease or disorder, or curing the disease or disorder. Similarly, as used herein, an “effective amount” or a “therapeutically effective amount” of a compound of the invention refers to an amount of the compound that alleviates, in whole or in part, symptoms associated with the disorder or condition, or halts or slows further progression or worsening of those symptoms or prevents or provides prophylaxis for the disorder or condition. In particular, a “therapeutically effective amount” refers to an amount effective, at dosages and for periods of time necessary, to achieve the desired therapeutic result. A therapeutically effective amount is also one in which any toxic or detrimental effects of compounds of the invention are outweighed by the therapeutically beneficial effects.

Phrases such as “under conditions suitable to provide” or “under conditions sufficient to yield” or the like, in the context of methods of synthesis, as used herein refers to reaction conditions, such as time, temperature, solvent, reactant concentrations, and the like, that are within ordinary skill for an experimenter to vary, that provide a useful quantity or yield of a reaction product. It is not necessary that the desired reaction product be the only reaction product or that the starting materials be entirely consumed, provided the desired reaction product can be isolated or otherwise further used.

By “chemically feasible” is meant a bonding arrangement or a compound where the generally understood rules of organic structure are not violated; for example, a structure within a definition of a claim that would contain in certain situations a pentavalent carbon atom that would not exist in nature would be understood to not be within the claim. The structures disclosed herein, in all of their embodiments are intended to include only “chemically feasible” structures, and any recited structures that are not chemically feasible, for example in a structure shown with variable atoms or groups, are not intended to be disclosed or claimed herein.

An “analog” of a chemical structure, as the term is used herein, refers to a chemical structure that preserves substantial similarity with the parent structure, although it may not be readily derived synthetically from the parent structure. A related chemical structure that is readily derived synthetically from a parent chemical structure is referred to as a “derivative.”

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described. Moreover, where features or aspects of the invention are described in terms of Markush groups, those skilled in the art will recognize that the invention is also thereby described in terms of any combination of individual members or subgroups of members of Markush groups. Thus, for example, if X is described as selected from the group consisting of bromine, chlorine, and iodine, and Y is described as selected from the group consisting of methyl, ethyl, and propyl, claims for X being bromine and Y being methyl are fully described.

If a value of a variable that is necessarily an integer, e.g., the number of carbon atoms in an alkyl group or the number of substituents on a ring, is described as a range, e.g., 0-4, what is meant is that the value can be any integer between 0 and 4 inclusive, i.e., 0, 1, 2, 3, or 4.

In various embodiments, the compound or set of compounds, such as are used in the inventive methods, can be any one of any of the combinations and/or sub-combinations of the above-listed embodiments.

In various embodiments, a compound as shown in any of the Examples, or among the exemplary compounds, is provided. Provisos may apply to any of the disclosed categories or embodiments wherein any one or more of the other above disclosed embodiments or species may be excluded from such categories or embodiments.

At various places in the present specification substituents of compounds of the invention are disclosed in groups or in ranges. It is specifically intended that the invention include each and every individual subcombination of the members of such groups and ranges. For example, the term “C1-C6 alkyl” is specifically intended to individually disclose methyl, ethyl, propyl, isopropyl, n-butyl, sec-butyl, isobutyl, etc.

For a number qualified by the term “about”, a variance of 2%, 5%, 10% or even 20% is within the ambit of the qualified number.

Standard abbreviations for chemical groups such as are well known in the art are used; e.g., Me=methyl, Et=ethyl, i-Pr=isopropyl, Bu=butyl, t-Bu=tert-butyl, Ph=phenyl, Bn=benzyl, Ac=acetyl, Bz=benzoyl, and the like.

A “salt” as is well known in the art includes an organic compound such as a carboxylic acid, a sulfonic acid, or an amine, in ionic form, in combination with a counterion. For example, acids in their anionic form can form salts with cations such as metal cations, for example sodium, potassium, and the like; with ammonium salts such as NH₄⁺ or the cations of various amines, including tetraalkyl ammonium salts such as tetramethylammonium, or other cations such as trimethylsulfonium, and the like.

A “pharmaceutically acceptable” or “pharmacologically acceptable” salt is a salt formed from an ion that has been approved for human consumption and is generally non-toxic, such as a chloride salt or a sodium salt. A “zwitterion” is an internal salt such as can be formed in a molecule that has at least two ionizable groups, one forming an anion and the other a cation, which serve to balance each other. For example, amino acids such as glycine can exist in a zwitterionic form. A “zwitterion” is a salt within the meaning herein. The compounds of the present invention may take the form of salts. The term “salts” embraces addition salts of free acids or free bases which are compounds of the invention. Salts can be “pharmaceutically-acceptable salts.” The term “pharmaceutically-acceptable salt” refers to salts which possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds of the invention.

Suitable pharmaceutically acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Examples of pharmaceutically unacceptable acid addition salts include, for example, perchlorates and tetrafluoroborates. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, laurylsulphonate salts, and amino acid salts, and the like. (See, for example, Berge et al. (1977) “Pharmaceutical Salts”, J. Pharm. Sci. 66: 1-19.)

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylenediamine, chloroprocaine, choline, diethanolamine, ethylenediamine, meglumine (N-methylglucamine) and procaine. Examples of pharmaceutically unacceptable base addition salts include lithium salts and cyanate salts. Although pharmaceutically unacceptable salts are not generally useful as medicaments, such salts may be useful, for example as intermediates in the synthesis of Formula (I) compounds, for example in their purification by recrystallization. All of these salts may be prepared by conventional means from the corresponding compound according to Formula (I) by reacting, for example, the appropriate acid or base with the compound according to Formula (I). The term “pharmaceutically acceptable salts” refers to nontoxic inorganic or organic acid and/or base addition salts, see, for example, Lit et al., Salt Selection for Basic Drugs (1986), Int J. Pharm., 33, 201-217, incorporated by reference herein.

Each of the terms “halogen,” “halide,” and “halo” refers to —F, —Cl, —Br, or —I.

A “hydroxyl” or “hydroxy” refers to an —OH group.

Compounds described herein can exist in various isomeric forms, including configurational, geometric, and conformational isomers, including, for example, cis- or trans-conformations. The compounds may also exist in one or more tautomeric forms, including both single tautomers and mixtures of tautomers. The term “isomer” is intended to encompass all isomeric forms of a compound of this disclosure, including tautomeric forms of the compound. The compounds of the present disclosure may also exist in open-chain or cyclized forms. In some cases, one or more of the cyclized forms may result from the loss of water. The specific composition of the open-chain and cyclized forms may be dependent on how the compound is isolated, stored or administered. For example, the compound may exist primarily in an open-chained form under acidic conditions but cyclize under neutral conditions. All forms are included in the disclosure.

Some compounds described herein can have asymmetric centers and therefore exist in different enantiomeric and diastereomeric forms. A compound of the invention can be in the form of an optical isomer or a diastereomer. Accordingly, the disclosure encompasses compounds and their uses as described herein in the form of their optical isomers, diastereoisomers and mixtures thereof, including a racemic mixture. Optical isomers of the compounds of the disclosure can be obtained by known techniques such as asymmetric synthesis, chiral chromatography, simulated moving bed technology or via chemical separation of stereoisomers through the employment of optically active resolving agents.

Unless otherwise indicated, the term “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound. The stereoisomer as described above can be viewed as composition comprising two stereoisomers that are present in their respective weight percentages described herein.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

As used herein, and unless otherwise specified, the term “compound” is inclusive in that it encompasses a compound or a pharmaceutically acceptable salt, stereoisomer, and/or tautomer thereof. Thus, for instance, a compound of Formula I includes a pharmaceutically acceptable salt of a tautomer of the compound.

The terms “prevent,” “preventing,” and “prevention” refer to the prevention of the onset, recurrence, or spread of the disease in a patient resulting from the administration of a prophylactic or therapeutic agent.

A “patient” or “subject” or “host” includes an animal, such as a human, cow, horse, sheep, lamb, pig, chicken, turkey, quail, cat, dog, mouse, rat, rabbit or guinea pig. In accordance with some embodiments, the animal is a mammal such as a non-primate and a primate (e.g., monkey and human). In one embodiment, a patient is a human, such as a human infant, child, adolescent or adult.

The term miRNA means a micro RNA sequence that is non-coding for peptides and functions at least for mRNA silencing and post-translational regulation of gene expression. Complementary base pairing of miRNA with messenger RNA molecules manages translation of the mRNA by up and/or down regulation, inhibition, repression and similar translation effects. Typical pre- and pri-miRNA sequences include structured and unstructured motifs. A structured motif is a segment of a pre- miRNA and its embedment within a pri-miRNA having a stable three-dimensional structure that is not wholly dependent upon the particular nucleotide sequence of the structure motif. Hairpin stem, bulge and/or terminal loop regions of pre-miRNA's are typical structured motifs. Groups of miRNAs often cooperate to manage mRNA function. An example is the pri-miRNA-17-92 cluster and the resulting pre-miRNA's and mature miRNA's produced by nuclease action on the cluster and pre-miRNA's respectively.

The terms pri-miRNA and pre-miRNA are the precursor RNA transcripts from which mature miRNA is produced. Transcription of DNA in the cell nucleus produces among other RNA molecules, pri-miRNA, a long RNA sequence which is capped and polyadenylated. Cleavage of the pri-miRNA and RNA chain processing in the nucleus produces the shorter pre-miRNA for export to the cellular cytoplasm. Pre-miRNA is further processed in the cytoplasm by RNAase Dicer to produce double stranded short RNA and one of the two strands becomes mature, single strand miRNA for interaction with messenger RNA.

The term “biologically silent” in the context of miRNA binding compounds or moieties means that the compound or moiety does not bind to a functional site of the miRNA molecule that is subject to enzymatic cleavage required for miRNA biogenesis. Typical functional sites of miRNAs are Dicer and Drosha processing sites. A synonym for biologically silent is biologically inactive in that a compound that binds with miRNA but is biologically inactive does not inhibit biogenesis of miRNA such as pri- and pre-miRNA to mature miRNA.

The term “aka” means also known as.

### DETAILED DESCRIPTION

As explained above, sequence-based targeting of RNA typically uses oligonucleotides that bind to an RNAs sequence and then recruit RNase H to cleave the RNA target.¹ This modality is generally best suited to target unstructured regions in an RNA, as molecular recognition occurs via base pairing.² RNA, however, plays a myriad of biological roles dictated by its diverse structures which control its function. 3,4 Small molecules are best suited to target highly folded regions as they can form complementary interactions in the pockets presented by an RNA fold.⁵ The coupling of small molecules to RNA structures alone does not necessarily cause an interaction that affects biological function.⁶ Therefore, the development of aspects of the invention are based upon several research focal points: i) to define avid interactions between small molecules and functionally silent RNA folds in human miRNAs and ii) to deploy RNA degrading modalities to elicit bioactive outcomes.

According to the invention, a number of molecular hits were developed through study of molecular recognitions in a target agnostic- and massively parallel-library versus library format between a diverse small molecule library and a library of three dimensionally folded RNA structures. These interactions produced a high resolution map between small molecule molecular structure and RNA three-dimensional structure binding and defined new chemotypes that avidly bind RNA. This interaction map was mined in a target-agnostic fashion across the folded RNA structures derived from the human genome to define avid molecular recognition events. Amongst thousands of interactions, a highly selective one between a novel RNA-binding small molecule and the precursor of disease-associated microRNA-155 (pre-miR-155). However, this binding interaction was biologically inactive as it does not bind to a functional site on pre-miR-155 in cells, i.e. a site that is subject to enzymatic cleavage required for miRNA biogenesis. Fortuitously, the binding site in pre-miR-155 is proximal to an RNA structure that has high potential to be cleaved by ribonuclease L (RNase L). To effect potent and selective biological activity the binding compound was appended with a second small molecule that binds to and activates RNase L to construct a ribonuclease targeting chimera (RIBOTAC). The RIBOTAC potently and selectively degrades pre-miR-155 in a variety of cell lines even at picomolar concentrations, selectively affecting disease-associated phenotypes in multiple cellular models. These studies illustrate that small molecule RNA-targeted degradation can be leveraged to activate biologically inactive binding events using RNA quality control machinery.

To accomplish the development of the interaction map, a 15,000 member natural product-like collection from the compound management and screening center (COMAS)⁷ was studied using a fluorescent indicator displacement (FID)⁸ assay in tandem with 2-Dimensional Combinatorial Screening (2DCS)⁹. This library was analyzed for binding to the 3-dimensional RNA folds presented in a 3×3 internal loop pattern with 4,096 members (3×3 ILL), probing 61,440,000 interactions. The FID assay identified 330 novel RNA binders (hit rate=2.2%) comprised of 14 novel scaffolds. Then, using 2DCS¹⁰ their RNA binding interactions were isolated and characterized, identifying six novel and selective RNA binding molecules (hit rate=0.04%) comprised of four novel chemotypes. These include bipyrrol pyrrolium salts, azolium salts, chromones, and 3-phenylfuro[3,2-b]pyridine-5-amines. These novel chemotypes were defined as new RNA binders as an analysis of the structural similarity between these compounds and all known RNA-binding small molecules by using Tanimoto coefficients.

A current gap in the field of small molecules targeting RNA is understanding how a small molecules structure influences its RNA binding preferences. To explore this avenue, 220 derivatives of readily available azolium salts were studied by 2DCS, identifying an additional 14 novel RNA binders (hit rate=6.4%). These chemotypes were confirmed to be unique relative to databases of known RNA binders such as Inforna¹¹ and R-BIND¹², as determined by calculating Tanimoto coefficients¹³ and comparing their physiochemical properties. Then, by utilizing LOGOs and DiffLOGO¹⁴ analyses of their RNA binding preferences, molecular similarity was shown to directly correlate with RNA sequence preferences. Moreover, novel chemotypes also expanded the known RNA-small molecule binding landscape, identifying 98 new RNA motifs that bind small molecules.

Leveraging this knowledge, the folds encoded by these miRNAs were then inspected for juxtaposition with potential ribonuclease targeting chimera (RIBOTACs)⁶ substrates which are rich in UA and AUU steps¹⁵, and disease relevant miRNAs.¹⁶ This identified precursor miR-155 as a potential target with clinical relevance in breast cancer¹⁷ and neurodegenerative inflammation.¹⁸ Using breast cancer we show that conjugation of RIBOTAC recruiters onto small molecules that bind functionally silent RNA folds, can convert them to potent bioactive compounds and alleviate disease phenotypes.

### Identification of Novel Natural Product Like RNA Binding Scaffolds

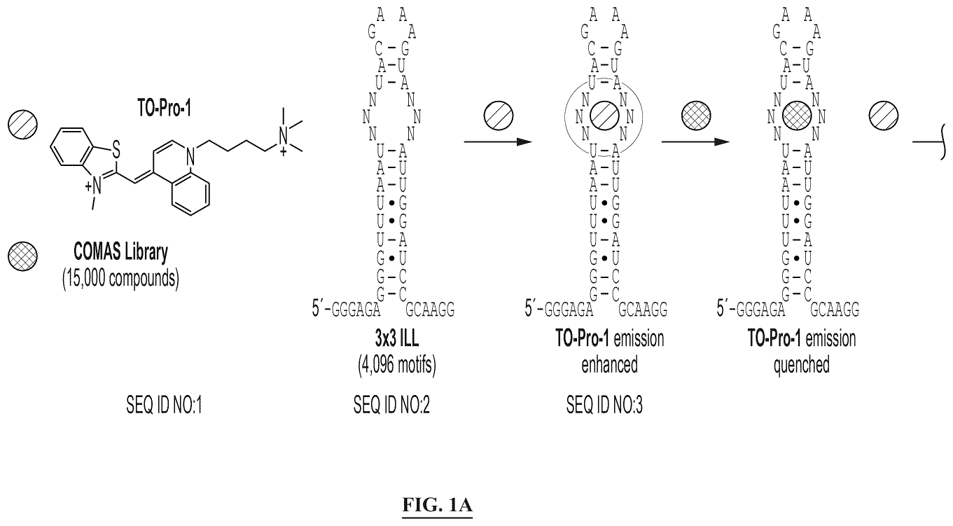

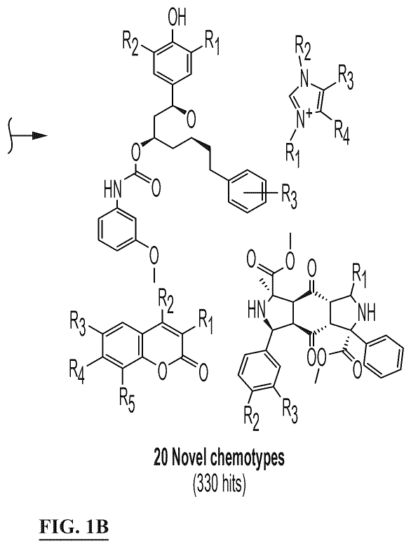

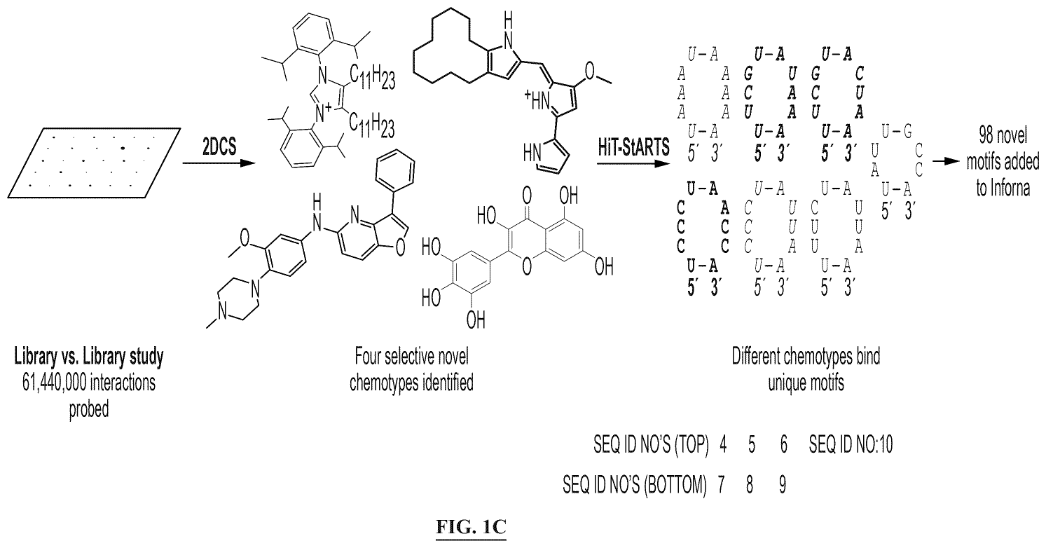

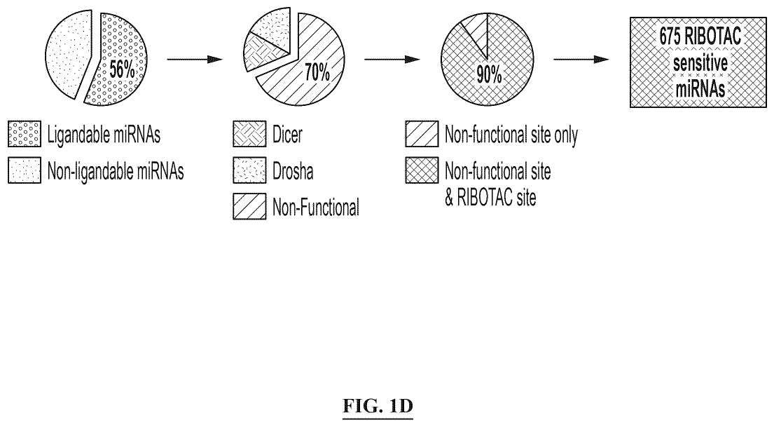

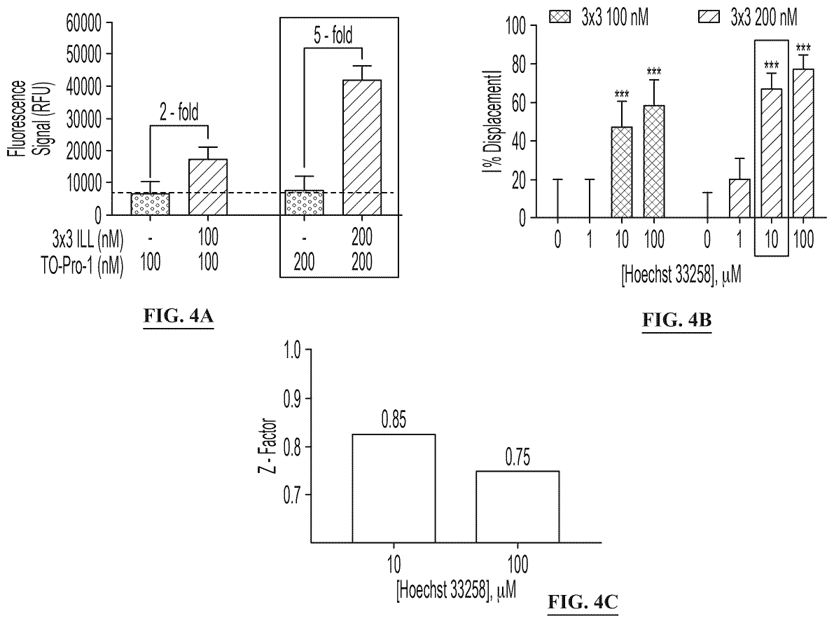

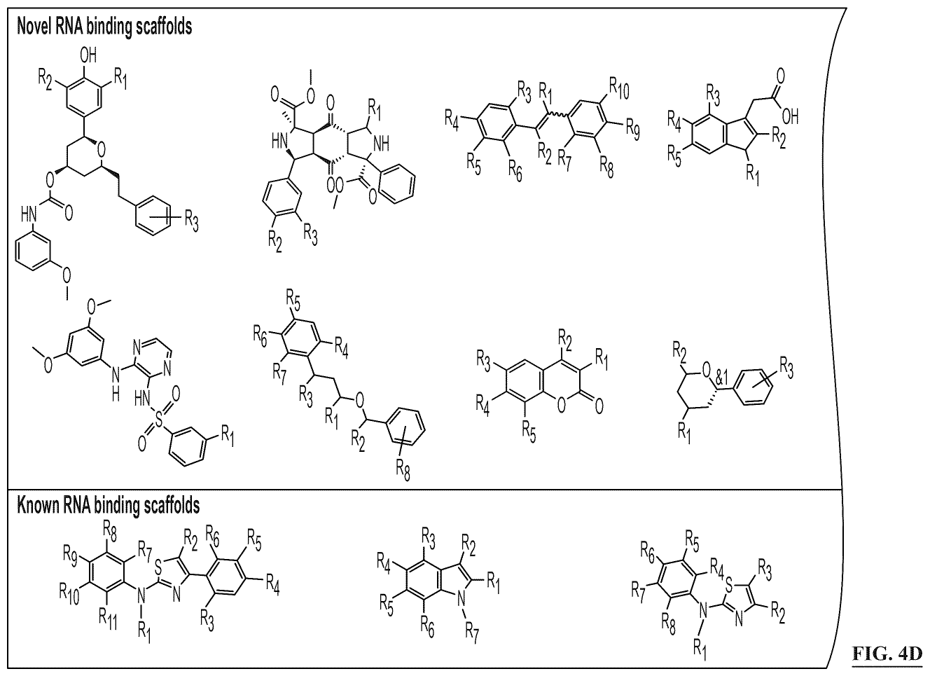

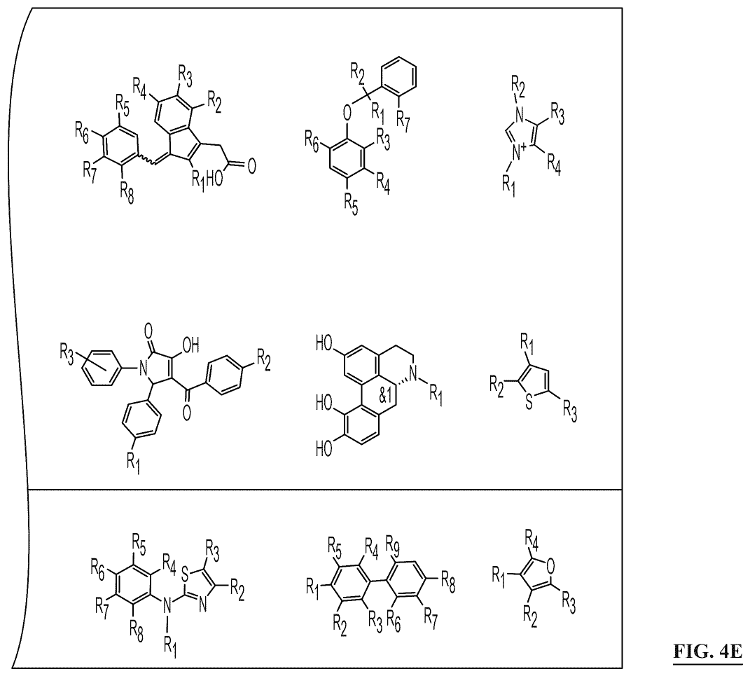

To identify novel RNA binders from natural product like scaffolds, a 15,000-member subset of the COMAS library was screened by in solution dye displacement assay using TO-Pro-1 bound to the 3×3 internal loop library. This library contains 4,096 unique RNA three dimensional folds, resulting in 61,440,000 RNA small molecule interactions being probed simultaneously (FIGS. 1A-1B). In this initial study, 330 molecules were identified (hit rate=2.2%) to dose dependently bind the RNA library. These molecules encompassed 20 unique scaffolds of which, 14 are novel (FIGS. 4A-4E).

The precise molecular recognition events of these 330 hits were then were studied by 2DCS¹⁰ to identify selective RNA binders. From these studies, six selective RNA binders (hit rate=1.8%) comprised of four novel scaffolds, including bipyrrol pyrrolium salts (C1, C4), chromones (C5), azolium salts (C2, C3) and 3-phenylfuro[3,2-b]pyridine-5-amines (C6), were identified. see FIG. 1C and Table 1 for structures. Because the goal was to study the effect of small molecule structure on RNA 3D fold binding preferences, an SAR library of 220 azolium salts were also studied for their RNA binding preferences by 2DCS. This afforded 14 additional azolium derivatives (C7-C20) as selective RNA binders (Table 1).

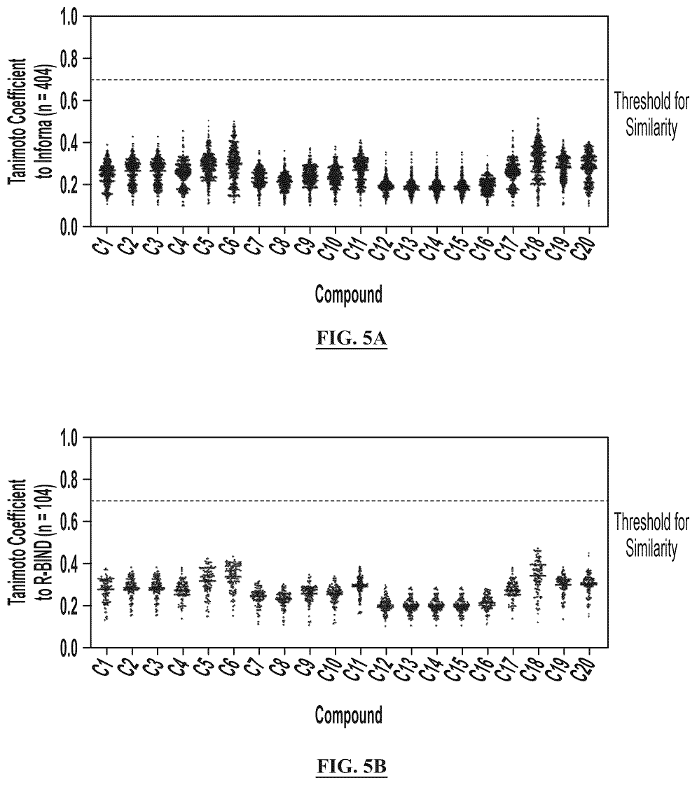

To confirm that these molecules were in fact unique, their similarity to compounds within Inforna¹⁹ and R-BIND¹², two databases of known RNA binders, was assessed by Tanimoto analysis (see footnote 13). This method scores molecules by chemical and spatial similarity and assigns them a coefficient ranging from 0 (being dissimilar) to 1(exactly the same). The analysis showed that compounds C1-C20 have a mean Tanimoto coefficient <0.4 indicating that these molecules are dissimilar to both databases and in fact are unique (FIGS. 5A-5B). To further corroborate this, we also analyzed their physiochemical properties and compared them to the molecules within Inforna and R-BIND. On average, compound C1-C20 exhibited higher cLogP (5.8 vs. 0.2) lower polar surface areas (33.4 Å² vs. 156.1 Å²) and fewer hydrogen bond donors and acceptors (donor: 0.5 vs. 5.2; acceptor: 1.4 vs. 8.6) than currently reported RNA binders (Table 2). The molecules also exhibited a higher number of rotatable bonds and aliphatic character, indicating they were less rigid than currently known compounds. Therefore, these molecules are structurally and chemically distinct from known RNA binding matter.

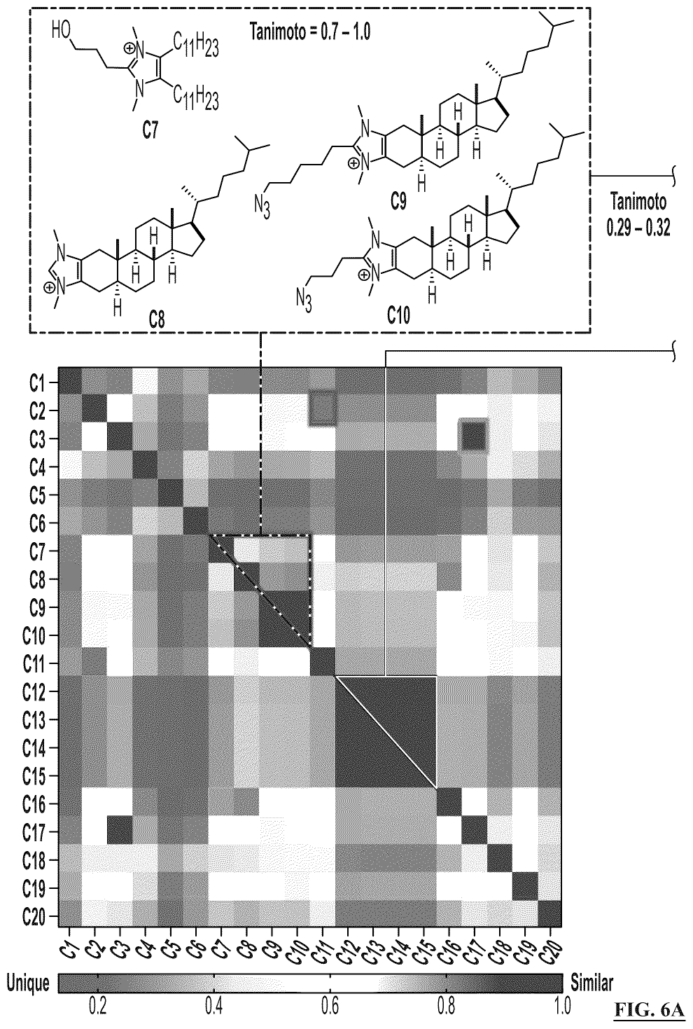

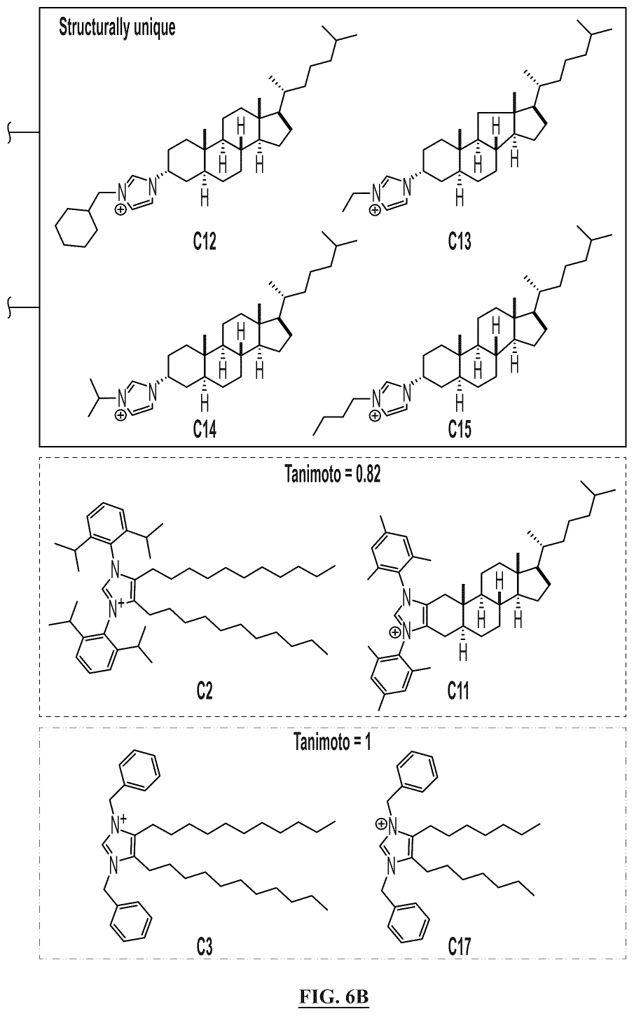

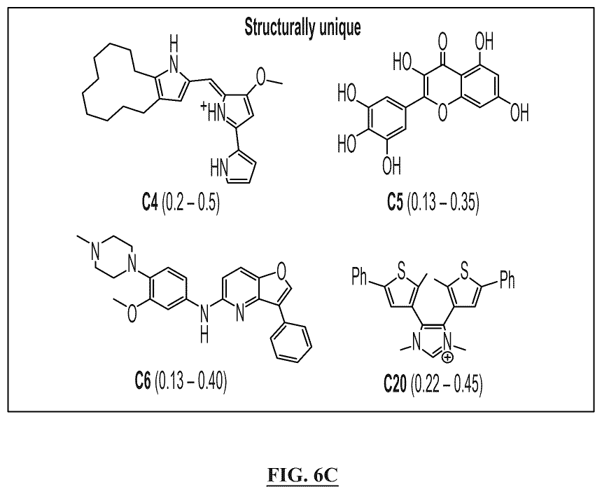

A Tanimoto comparison of C1-C20 among themselves indicated clustering of the hits into three distinct groups with Tanimoto scores ranging from 0.7 to 1.0. Compounds C7, C8, C9 and C10 clustered together and exhibit highly aliphatic functionalities such as n-undecyl (C7) cholesteryl groups (C8-C10) fused to the azolium core. Interestingly, compounds C12-C15 also share their cholesteryl moiety, however, instead of the connectivity being a fused ring, the cholesteryl group is appended on one of the azolium nitrogen's. This change in connectivity makes C12-C15 spatially distinct from C7 -C10 with 11their Tanimoto scores ranging from 0.29-0.32 (FIG. 6A-6C). Other molecules with high similarity include compounds C2 and C11 which are chemically different based on their 2D structure, however Tanimoto analysis indicates they are spatially very similar. Molecules which exhibited spatial and chemical distinctiveness compared to all others include C4, C5, C6, C7, and C20, which had mean Tanimoto scores <0.5 to all other compounds in the series (FIGS. 6A-6C). After establishing the chemical uniqueness of these molecules, we next assessed their RNA binding preferences.

### Novel Chemotypes Exhibit Unique RNA Fold Preferences.

To understand the effect of chemical structure on a small molecule's RNA binding preferences, 2DCS was used along with High-throughput Structure Activity Relationships Through Sequencing (HiT-StARTS)¹⁰ as previously described to annotate and rank each compounds RNA binding preferences by affinity.^{19, 10} This information was then deposited into Inforna for cross referencing to known RNA folds.

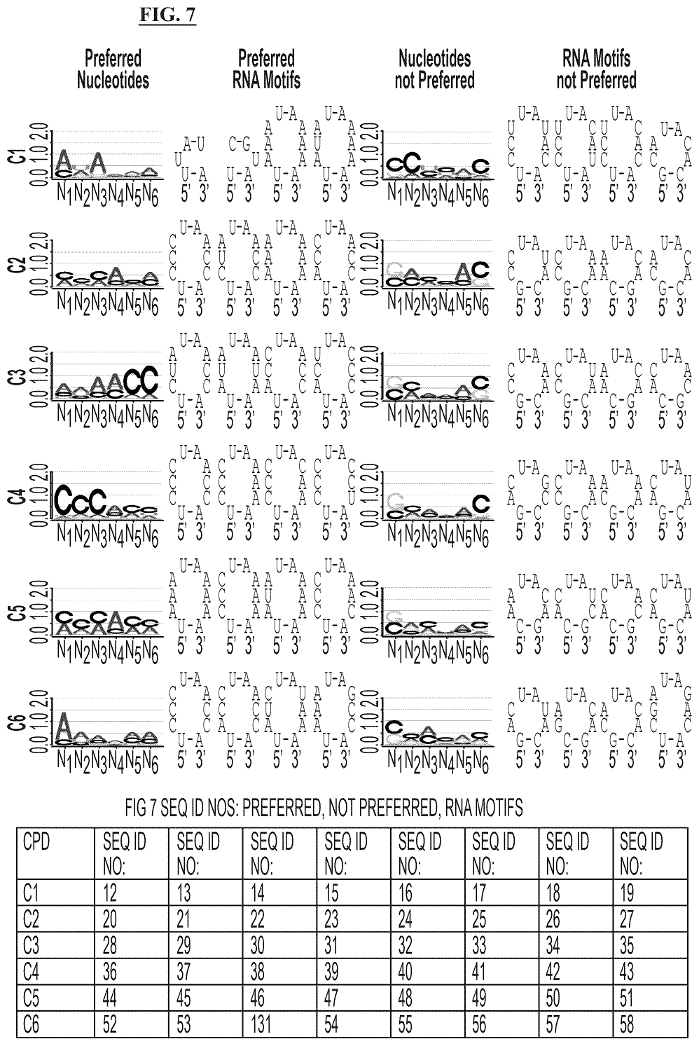

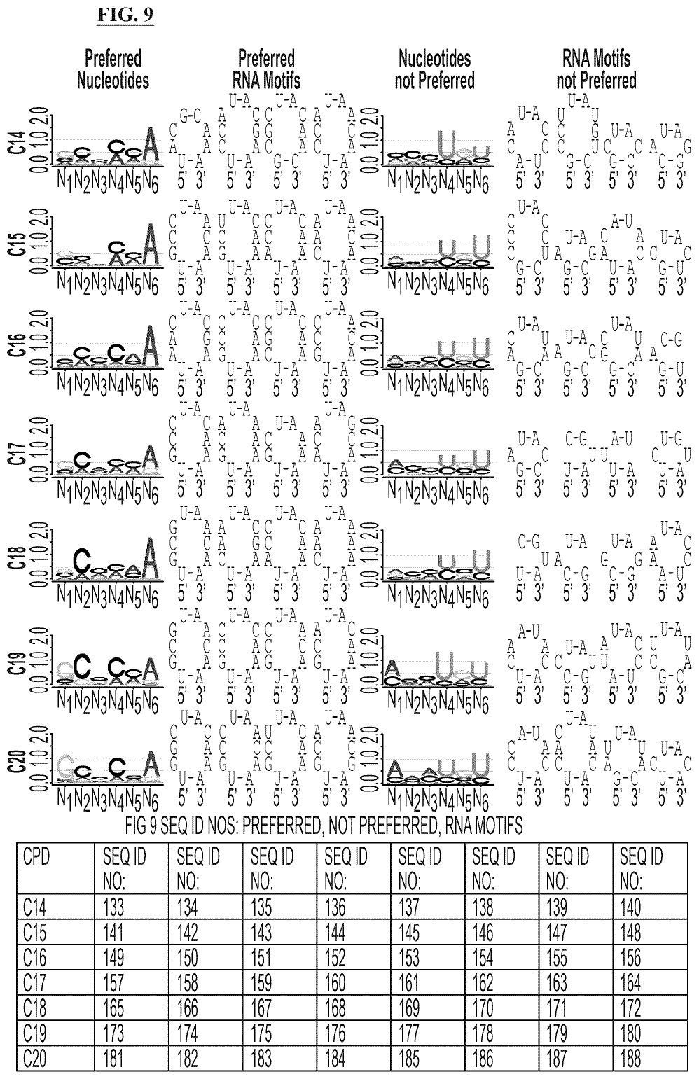

To rapidly assess trends in their RNA fold preferences the top 0.5 and bottom 0.5% of RNA motifs were analyzed by LOGOS and DiffLOGOS¹⁴ as previously described.²⁰ This revealed that globally the molecules preferred 3×3 internal loops, which comprised 90% of all motifs, followed by 2×2 IL (5%), single nucleotide bulges (3.8%) and 1×1 internal loops (1.2%), see FIGS. 7-9.

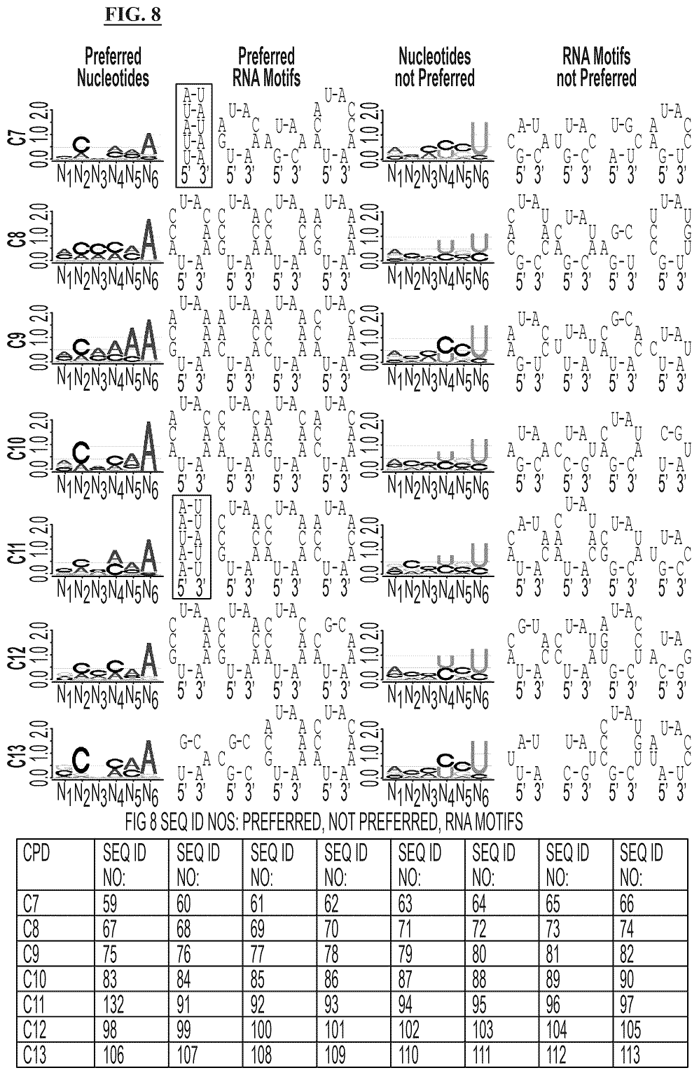

Interestingly, while it is known that sequences within the 3×3 ILL can form fully base paired motifs, previous 2DCS studies have never identified them due to 2DCS's highly stringent competitive binding analysis. However, even with such stringent conditions, compounds C7 and C1l were identified to bind alternating (AU)(UA) pairs as their highest affinity interactions, with a fitness score of 100% (FIG. 8). The next motifs after these had Fitness Scores of 46 and 56% respectively, indicating that the next significant interactions were much weaker.

Such base pair binding compounds are of tremendous utility in RNA structure targeting applications as RNA is˜50% base paired^{21,22} and that RNA folds can be separated by long stretches of base pairs, making multivalent ligands targeting only internal loops, bulges etc. intractable. This is exemplified in the targeting of precursor miRNA-200c, which required generation of an internal loop-base pair targeting hybrid to afford a potent and selective inhibitor of the miRNA.²³

Given that the 3×3 ILL was used in this study, an enrichment of 3×3 internal loops might be expected. However, 3×3 internal loops comprise only 35% of all of the motifs in the library with 1×1 IL, 2×2 internal loops and bulges comprising 25, 29 and 11% respectively. An analysis of the enriched motifs for C1-C20 showed that 56% of the motifs were 3×3 internal loops, 21% more than the 3×3 internal loops (p<0.00001, 99% CI). Similarly, 2×2 internal loops comprised 25% of the enriched motifs compared to 29% for the 3×3 internal loops (p<0.0001, 99% CI). All other motifs did not show a statistically significant differences between what was observed for the 3×3 ILL and the enriched motifs for C1-C20 (See Table 3).

To confirm that these differences are due to the chemical uniqueness of C1 -C20, the motif distributions observed for molecules reported by Haniff et al.²⁴ using the 3×3 ILL were compared against those identified herein.¹ This analysis showed that in previous 2DCS analysis using the 3×3 ILL, 3×3 internal loops comprised only 15% of all the enriched motifs, compared to 56% for the molecules identified herein (p<0.000001, 99% CI). Similarly, compounds C1-C20 favored 2×2 internal loops by 2-fold compared to previous studies²⁴ (29% vs. 14%; p<0.00001, 99 CI). Interestingly, lxi internal loops were significantly dis-enriched by 2-fold (59% vs. 29%) for the data reported by Haniff et al.²⁴ compared to these studies (p<0.000001, 99% CI). Neither study show a significant deviation for their enrichment of bulged loops (Table 3).²⁴ To understand this, the physiochemical properties of C1-C20 were compared to those reported by Haniff et al.²⁴ The molecules C1-C20 exhibited 3-fold more aliphatic atoms, -2-fold fewer aromatic atoms, 2.7-fold lower polar surface area, and 1.5-fold higher cLogP than the molecules previously reported (Table 2).²⁴ This indicates that the compounds are less rigid due to their higher aliphatic and lower aromatic character and thus occupy a larger 3D volume necessitating larger motifs to accommodate them. ¹ All of the molecules within Inforna, including those reported by Haniff et al. are chemically dissimilar to C1 -C20 as they have Tanimoto coefficients <0.4 (FIGS. 5A and 5B).

Non-Functional RNA Motifs are Prevalent in the Human miRnome and Inactive Small Molecules that Binding them can be Optimized for Bioactivity by Conversion to Ribonuclease Targeting Chimeras (RIBOTACs).

A major gap in RNA small molecule targeting is the inability to target RNAs which lack ligandable functional sites, i.e., Dicer and Drosha sites in miRNAs.⁶ Numerous studies have shown that simple binding can yield highly potent bioactive interactions with miRNAs²⁴⁻²⁸ and when conjugated with RNA degrader modules such as Bleomycin²⁹ and RIBOTACs³⁰, their potency is enhanced by >10-fold.³⁰⁻³³ A RIBOTAC, involves the conjugation of a simple binder with a module that recruits and dimerizes Ribonuclease L (RNase L), activating it locally within a cell to cleave an RNA transcript and decrease its expression levels. The advantage of RIBOTAC conjugates as enhancers of activity are many fold, such as i) catalytic cleavage of the RNA; ii) specific degradation of UU and UA rich motifs; iii) formation of a ternary complex that spatially restricts cleavage.⁶ Previous studies of miR-21³⁰ and miR-96³¹ using RIBOTAC have enhanced the efficacy of simple binders to functional sites, however, not all miRNAs contain sites functional sites that are sensitive to simple binding.

To understand the scope of miRNAs capable of being targeted by compounds C1-C20, their bound motifs were compared to all motifs found within human miRNAs (1,917 miRNAs). This identified that 56% (1,075 miRNAs) were targetable by C1-C20. These sites were then classified by whether they occupied Dicer, Drosha, or non-functional sites within each hairpin precursor, revealing that 70% of the motifs (750 miRNAs) are non-functional and have a small molecule that targets them. As, mentioned earlier, RIBOTAC sites are rich in UU and UA steps, however, little is known about the exact nature of the most efficient substrates of RNase L.

The known RIBOTAC cleavage sites reported for miR-21³⁰ and miR-210²⁶ which showed at least 50% UA content were therefore analyzed. Using this as a cutoff, the number of non-functional ligandable miRNAs that contain at least one motif with a potential RIBOTAC cleavage site were assessed to afford 675 miRNAs, including miR-21 and miR-210. Thus, 90% of miRNAs with a targetable non-functional site are also potential RIBOTAC substrates (FIG. 1D).

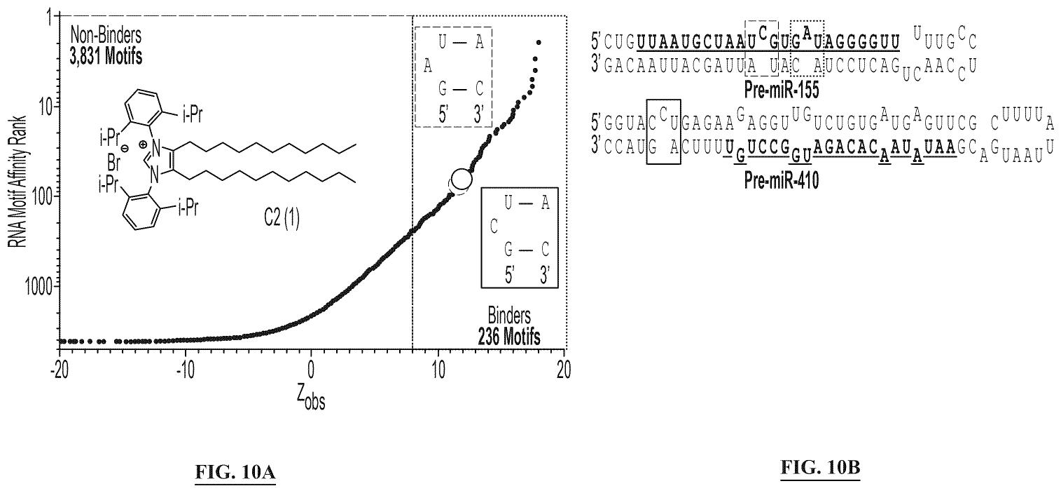

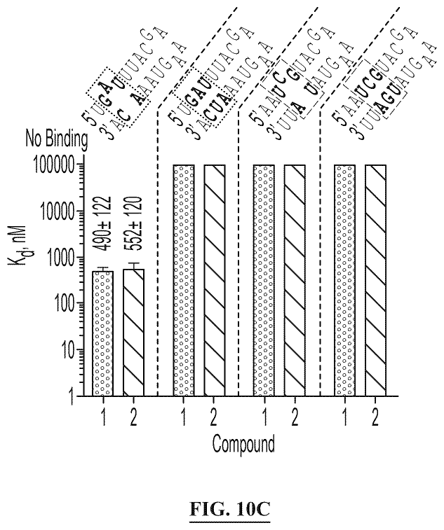

Cross referencing these miRNAs with known disease associated miRNAs housed within the Human microRNA Disease Database (HMDD v3.0)¹⁶ revealed that C2 targeted non-functional sites in pre-miR-155 and pre-miR-410 (FIGS. 10A-10C). Due to miR-155s broad implication in multiple disease settings including cancer³⁴, neuroinflammation¹⁸ and neurodegenerative¹⁸ disorders like Alzheimer's disease this miRNA was selected for further study. Henceforth, compound C2 will be referred to as compound 1, e.g., Compound C2(1) or simply compound 1.

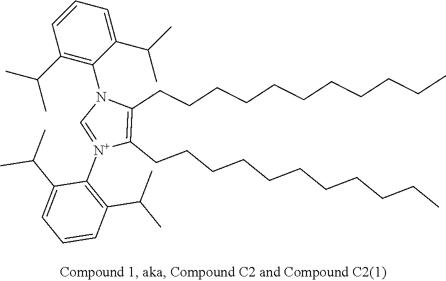 [SMILES: CCCCCCCCCCCc1c(CCCCCCCCCCC)[n+](-c2c(C(C)C)cccc2C(C)C)cn1-c1c(C(C)C)cccc1C(C)C]

### Compound 1 Binds to pre-miR-155's A Bulge And Is Activated By Conversion To A RIBOTAC degrader

Before conversion to a RIBOTAC, compound 1's affinity to pre-miR-155's 5′GAU/3′C_A bulge was measured by microscale thermophoresis (MST). This afforded a Ka of 490±122 nM with no binding to a base paired control RNA. Compound 1 however, lacks a functional handle for conjugation of the RIBOTAC recruiter, and was modified to replace its -n-undecyl alkyl chains with a propionic acid linker, Compound S5, see experimental section and the compound of Formula II depicted below. In order to mimic conjugation, this linker was amidated with 1-propylamine to afford control Compound 2, depicted below (Formula III).

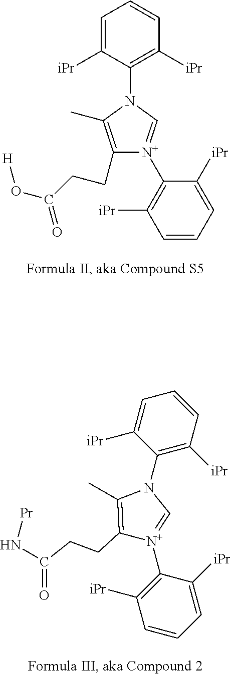

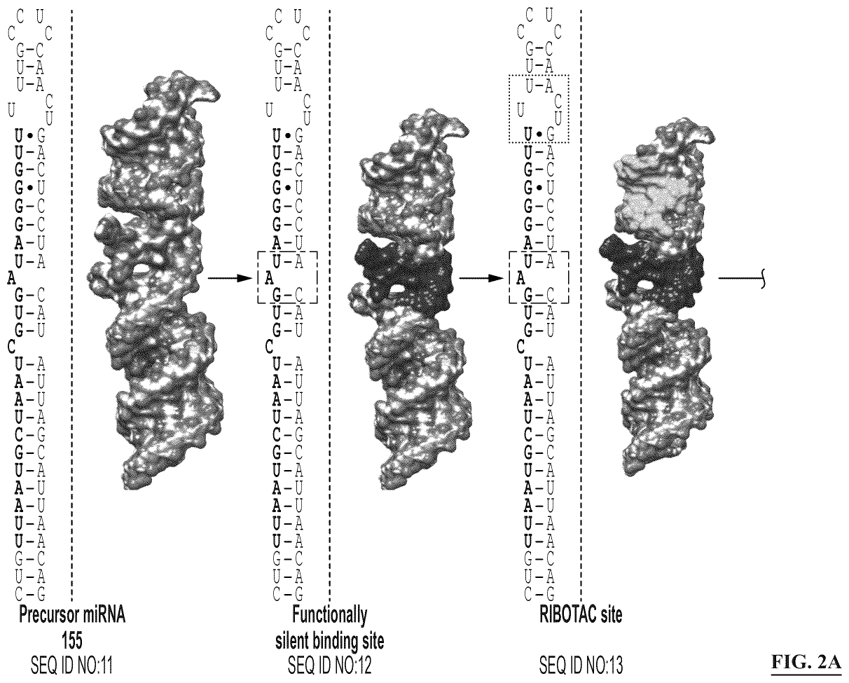

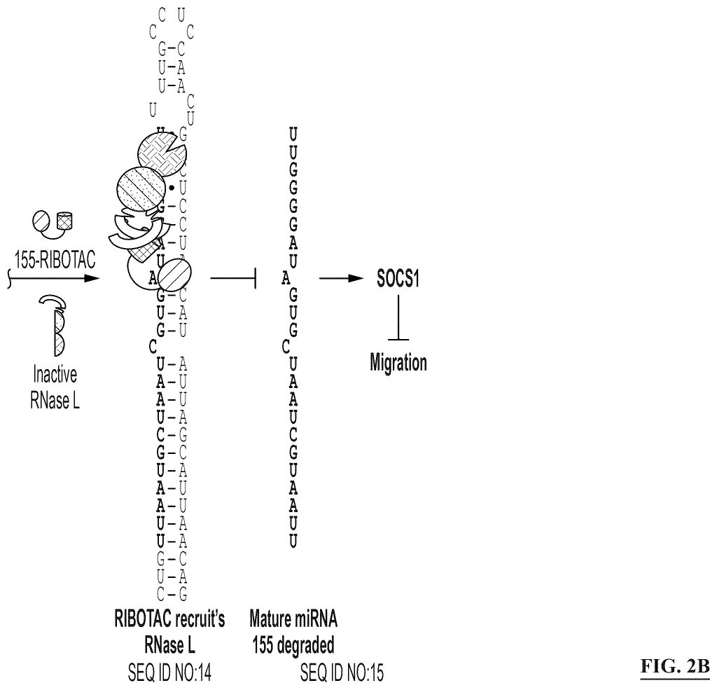

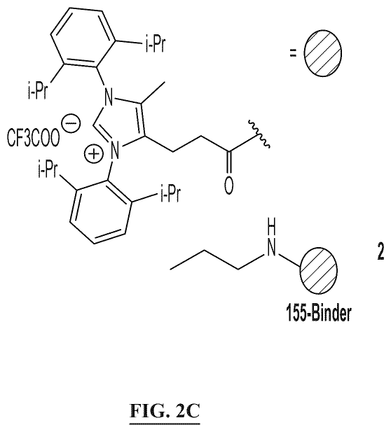

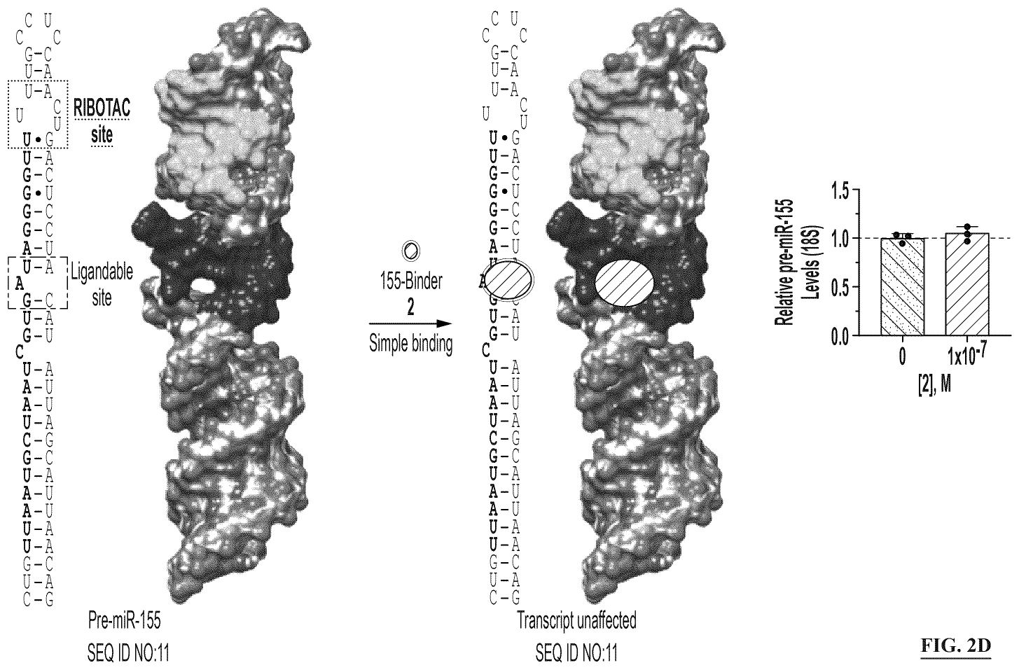

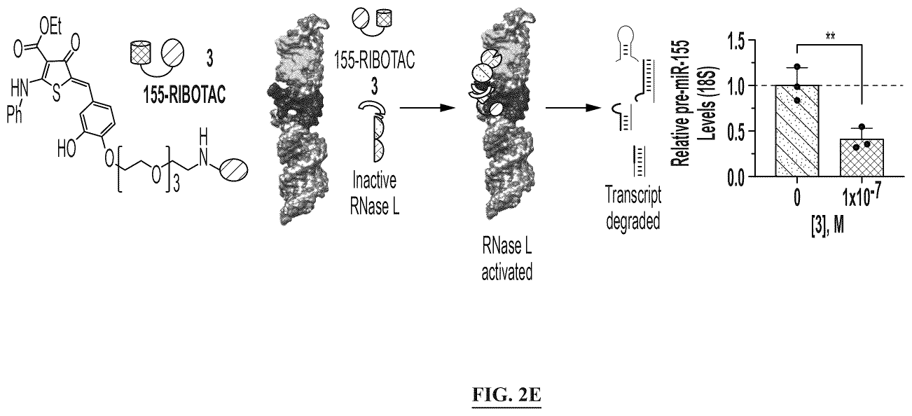

To determine whether this modification affected the molecules RNA binding affinity, the binding of Compound 2 to the A bulge and a base paired control were measured. Compound 2, bound with a Ka of 552±120 nM, with no affinity to the base paired control (FIG. 2C). Neither compound (1 or 2) had any affinity to pre-miR-155's C-bulge and GC paired control as expected because Inforna did not predict the 5′UCG/3′A_U bulge as a binding partner to 1. Also, removal of the alkyl chains did not significantly alter its affinity (FIG. 10C).

As a result of 2's binding to a non-functional site, the molecule is unable to elicit a bioactive interaction to inhibit miR-155 biogenesis or reduce mature miR-155 levels. To activate it, the acid precursor of 2, Compound S5, Formula II, was conjugated with the RIBOTAC recruiter module C13³⁰ to afford compound 3 (Formula I) depicted below.

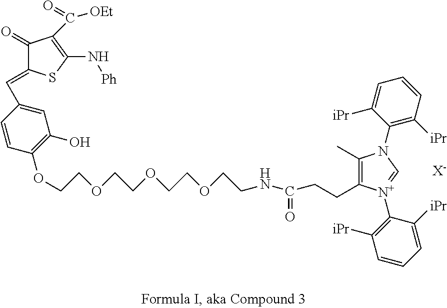

As a control, the RIBOTAC recruiter that is not conjugated to the RNA binding module S5 was used, which should not trigger specific cleavage of pre-miR-155 as the molecule will be unable to engage the RNA. This compound is referred to as compound 4 (see the experimental section for the structure of compound 4).

pre-miR-155 was then incubated with compound 3 in vitro in an RNase L cleavage assay. Addition of compound 3 triggered dose dependent cleavage of pre-miR-155 at residues U28 to U30 with an IC₅₀ of ˜1×10⁻⁷ M (FIG. 11A), which corresponded to the 5′ side of the predicted cleavage site (5′UUU/3′GUCA). To study if compounds 1 and 3 bound to the same site, pre-miR155 was cotreated with compound 1 in dose response (1×10⁻⁷ -1×10⁻⁴ M) and constant concentration of 3 at 1×10⁻⁷ M. This resulted in dose dependent inhibition of pre-miR-155 cleavage by RNase L, with an IC₅₀ 1.9±0.5 RM, indicating that 1 and 3 compete for binding to the same site (FIG. 11B).

To assess the site specificity of cleavage for RNase L pre-miR-155's RIBOTAC site was mutated to AU and GC base pairs. Treatment of the mutated pre-miR-155 with 3 and RNase L had no effect on the miRNA, indicating that this site must be single stranded to be accessible for RNase L cleavage.

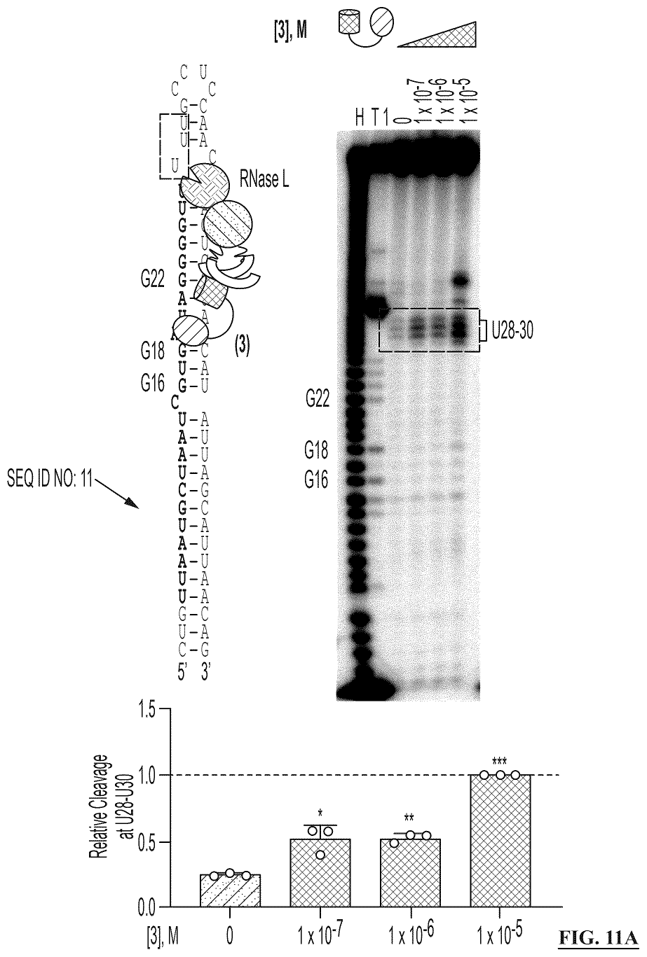

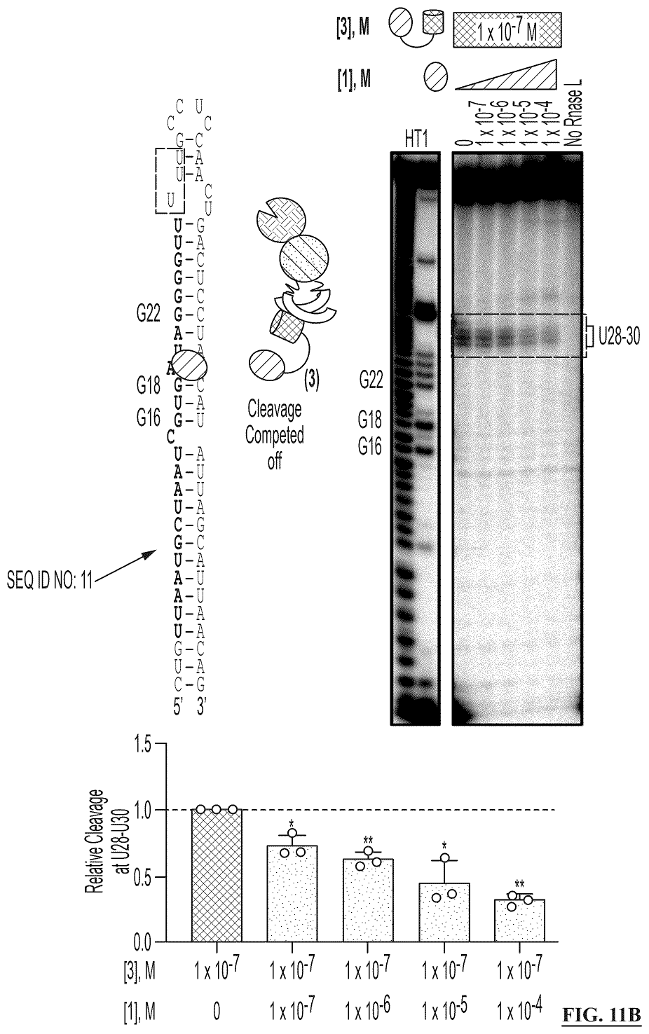

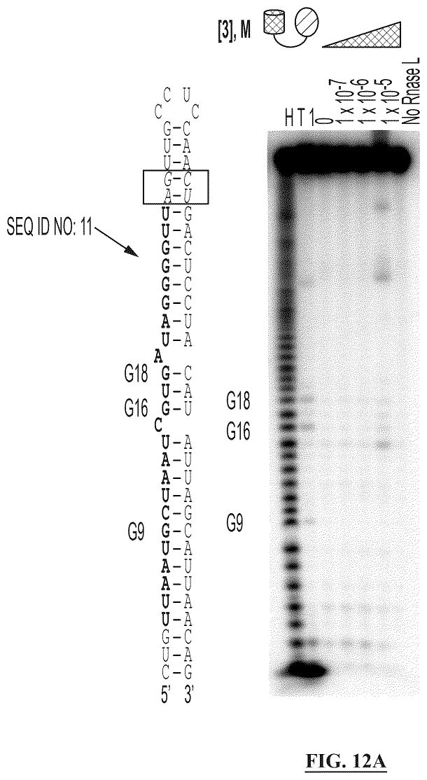

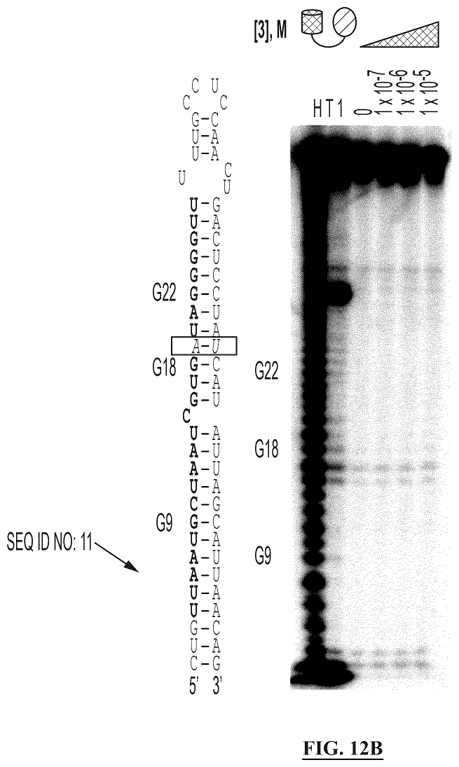

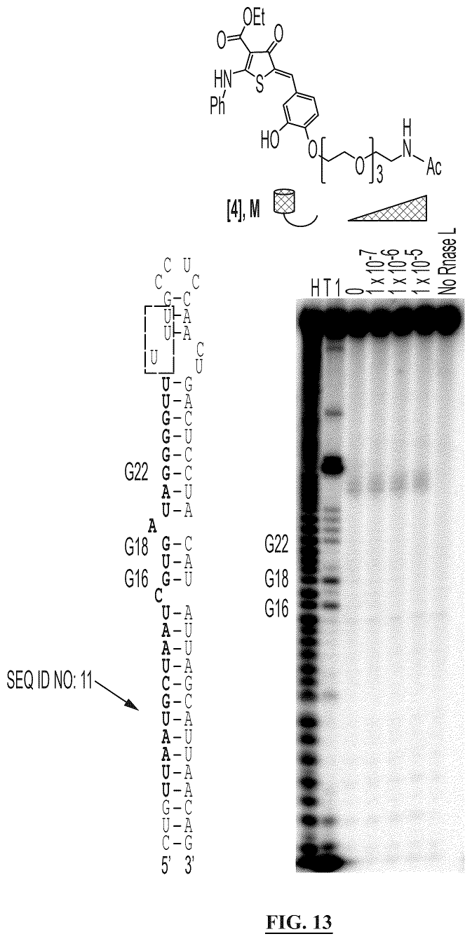

To ensure that the activity of 3 is due to the RNA binding module engaging the 5′GAU/3′C_A bulge, this site of pre-miR-155 was next mutated to an AU base pair. As shown earlier this mutation ablated binding of compound 2 (Formula II), and as shown in FIG. 12B, this mutation ablated cleavage by compound 3 (Formula I). This suggests that for on target cleavage the RIBOTAC molecule must directly engage the miRNA, similar to previous observations.^{26,30,31} This observation was confirmed by incubation of pre-miR-155 with control compound 4, the RIBOTAC recruiter lacking the RNA binding module, which resulted in no significant cleavage (FIG. 13).

### Compound 3 (Formula I) Selectively Degrades pre-miR-155 in MDA-MB-231 cells in an RNase L-dependent manner

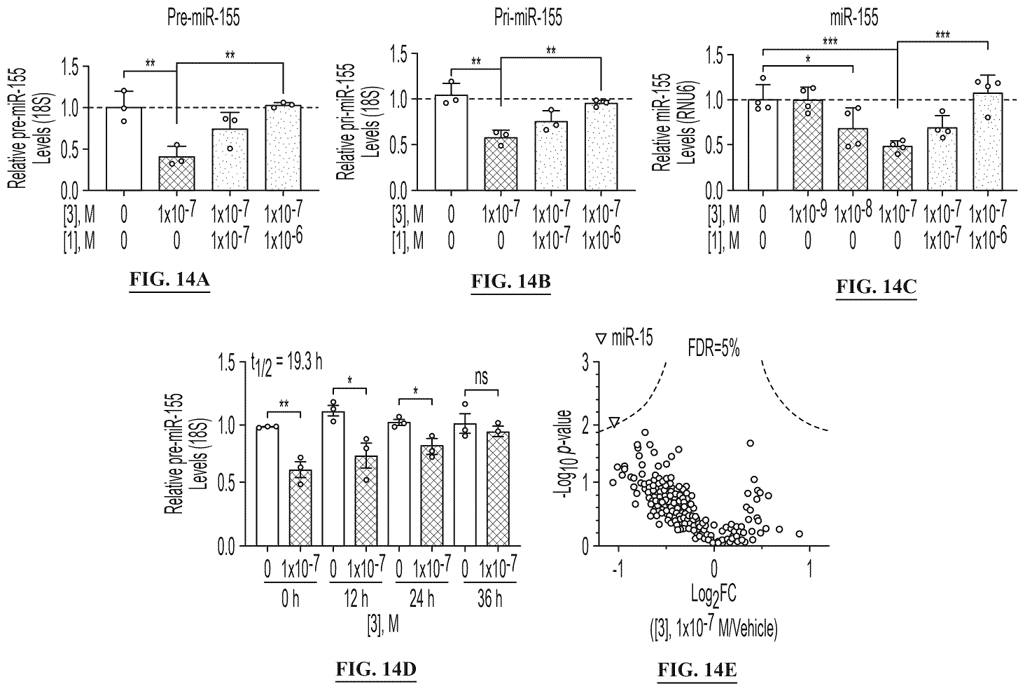

The next determination considered whether activation of the 5′GAU/3′C_A site also occurred in cells. MDA-MB-231 cells were treated with compound 2 or 3 (Formulas III and I respectively) and the levels of mature, pre and pri miR-155 were measured by RT-qPCR. Compound 2 (no recruiter moiety) had no effect on pre-miR-155 levels (FIG. 2D). However, compound 3 (Formula I) resulted in degradation of pre-miR-155 by 60% at 1×10⁻⁷ M (FIG. 2E and FIG. 14A). Since the 5′GAU/3′C_A bulge is also present in pri-miR-155, The ability of compound 3 (Formula I) to cleave the primary transcript was measured. These studies showed that compound 3 (Formula I) degraded the primary transcript by 43% (FIG. 14B). This lower efficacy is likely due to the low concentrations of RNase L in the nucleus.³⁵ Analysis of mature miR-155 levels corroborates this showing dose dependent cleavage from 1×10⁻⁹ (1 nanomolar) to 1×10⁻⁷ M (0.1 micromolar) (FIG. 14C). To confirm if compound 1 and 3 bind to the same site, MDA-MB-231 cells were co-treated with 1 in dose response and 3 at 1×10⁻⁷ M. Co-treatment with compound 1 attenuated cleavage by 3 dose dependently, with complete ablation at 1×10⁻⁶ M for all three forms of miRNA (mature, pre- and pri-) (FIGS. 14A-14C).

The half-life and selectivity of the cleavage effects of compound 3 (Formula I) in MDA-MB-231 cells were studied. After dosing cells with compound 3 for 48 h, compound 3 was removed, and total RNA was harvested at 0, 12, 24 and 36 h post-removal of compound. RT-qPCR analysis showed that pre-miR-155 levels returned to normal at 36 h, and a linear regression analysis of this data calculated a half-life of 19.3 h for the effect of compound 3 to be reduced by 50%. This corresponds well with reported half-lives of miR-155 which range between 12.4³⁶ and 28³⁷ h depending on the RNAs location (FIG. 14D).

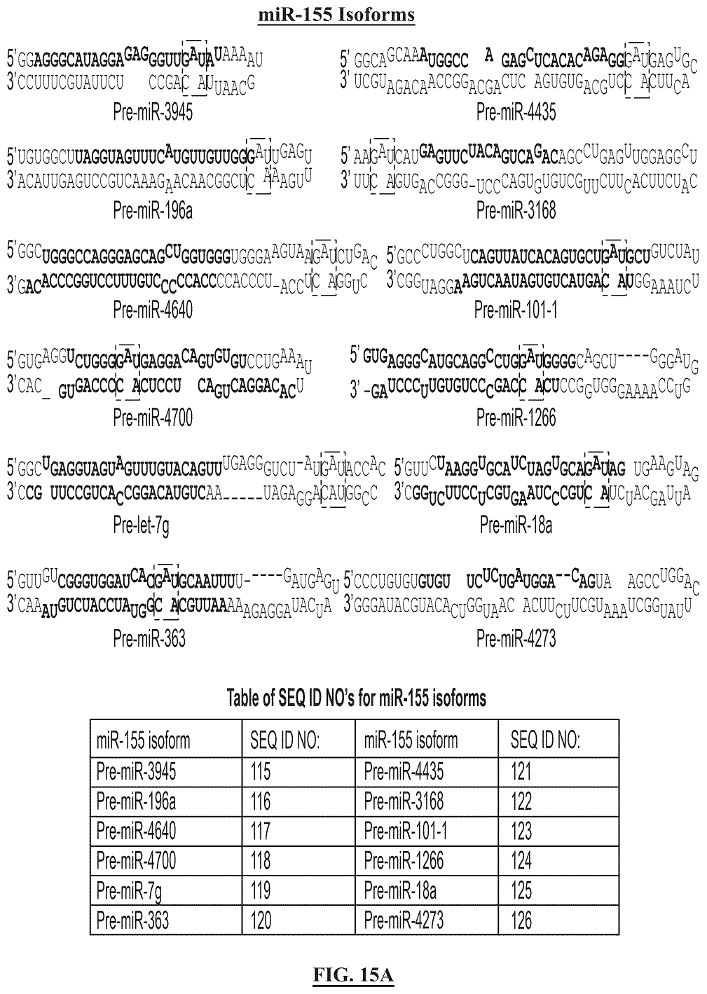

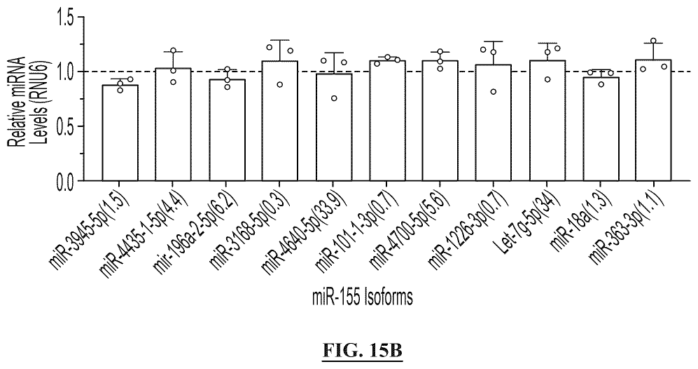

Next, the selectivity of 3 to inhibit miR-155 out of 373 expressed miRNAs in MDA-MB-231 cell was assessed. Mature miRN-155 was the most significantly downregulated amongst all miRNAs studied (FIG. 14E). Since multiple RNAs can also exhibit the same binding site as 3 other human miRNAs that contain the 5′GAU/3′C_A binding site were searched and 12 other RNAs dubbed RNA isoforms²⁷ were identified (FIG. 15A). A comparison of their structures to the aforementioned list of miRNAs with potential RIBOTAC sites identified that all except pre-miR-101-1 and pre-let-7g have a RIBOTAC targetable motif. However, RT-qPCR revealed that all of these isoforms were unaffected by 3 (FIG. 15B). This suggests that other factors beyond UU or UA content influence how suitable an RNA is as a substrate for RNase L cleavage.

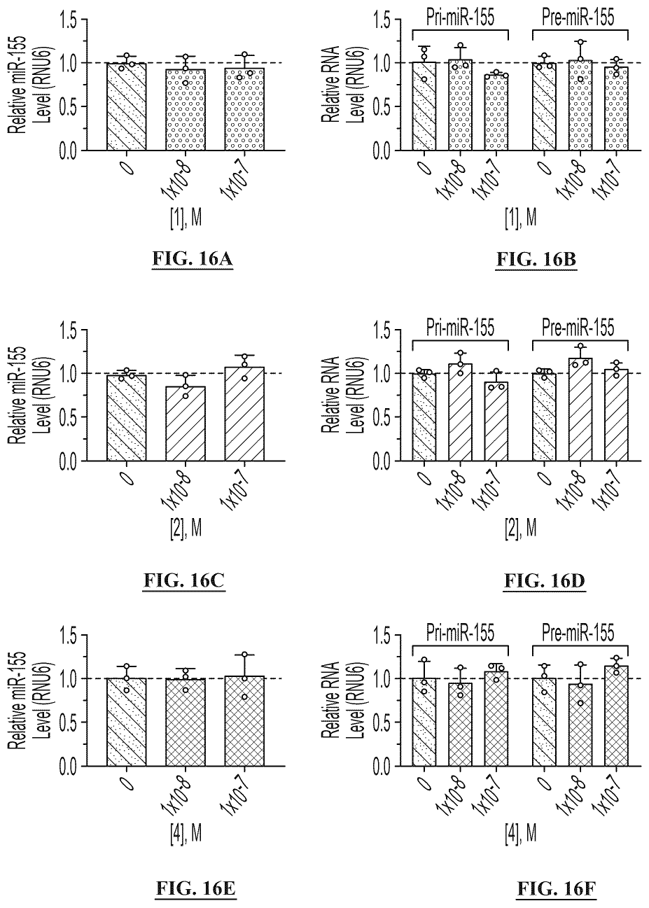

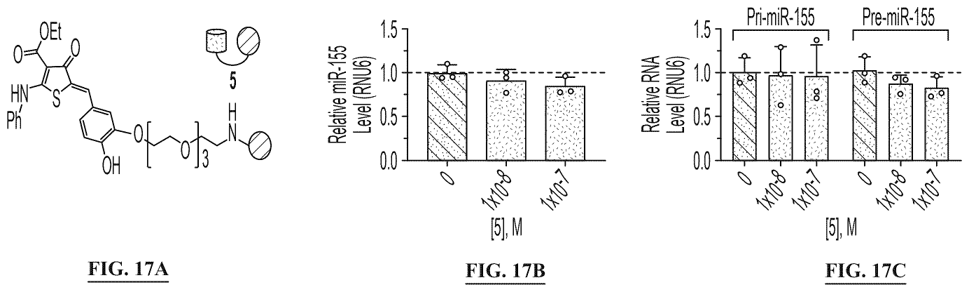

Lastly, the effects of the simple binders 1 and 2, and the inactive RIBOTAC probes 4 (no RNA binding module) and 5 (inactive RNase L recruiter module) on miR-155 levels were studied. These analyses showed that all four molecules had no effect on miR-155 levels at all tested concentrations (FIGS. 16A-17C).

### Compound 3 (Formula I) Directly Engages pre-miR-155 and RNase L in Cells to Elicit Bioactivity

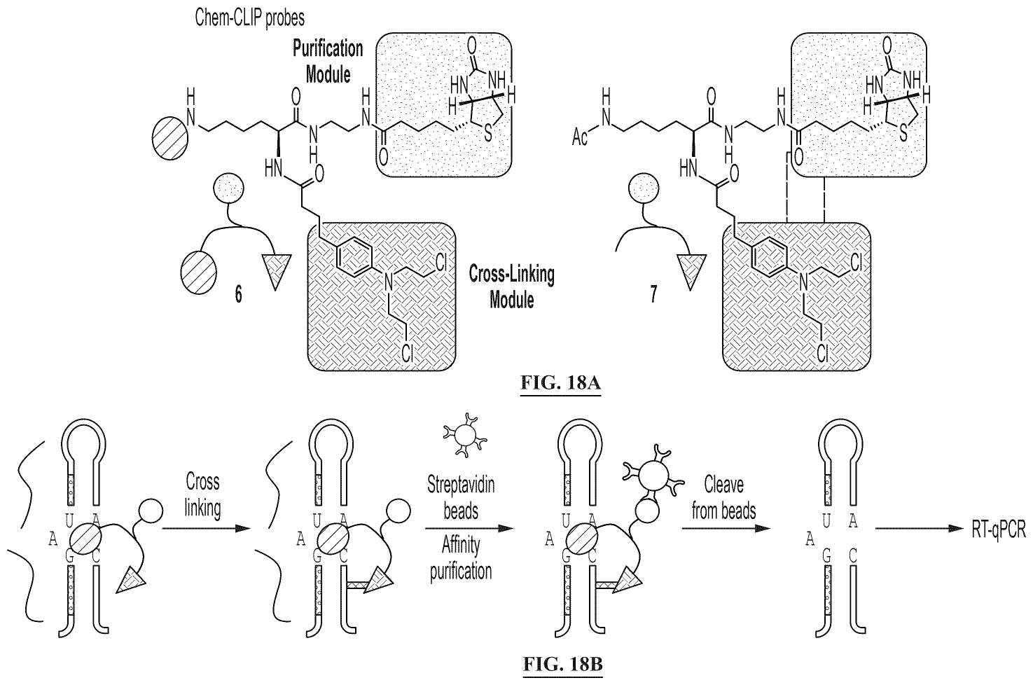

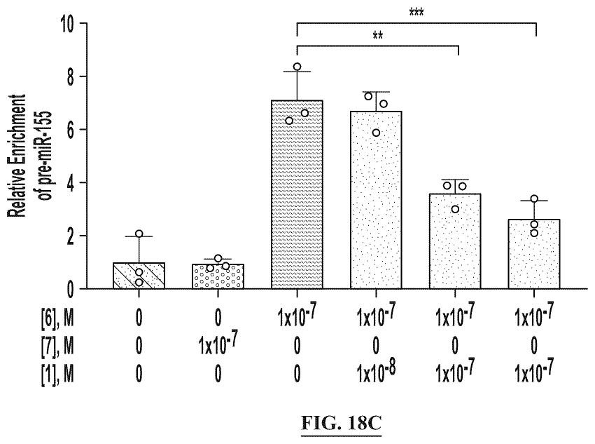

After establishing that 3 (Formula I) decreased levels of miR-155, its mode of action was studied. Using Chemical-Crosslinking and Isolation by Pulldown (Chem-CLIP) and competitive Chem-CLIP (C-Chem-CLIP) we studied the engagement of a derivative of compound 2 (Formula III) with pre-miR-155. To do this, we conjugated the carboxylic acid precursor of 2, RNA binding module S5/Formula II, with a reactive module, chlorambucil (CA), and a pull-down handle (biotin) to allow for reaction with bound RNAs and enrichment, respectively, affording compound 6 (Formula VI) and a control compound 7 that lacks the RNA binding module, (FIGS. 18A-18C). Control compound 7 depicted in the experimental section was synthesized by substituting an acetyl group for the Formula II moiety of Formula VI to provide an acetamido group at the left side of Formula VI instead of the Formula II moiety.

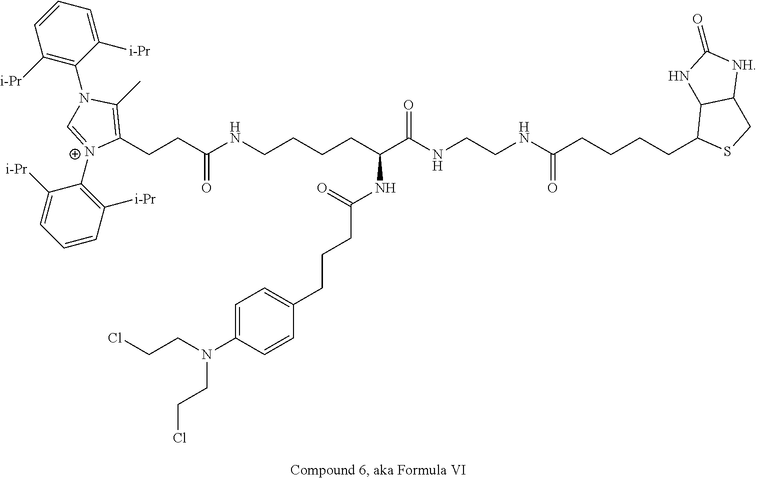 [SMILES: Cc1c(CCC(=O)NCCCC[C@H](NC(=O)CCCc2ccc(N(CCCl)CCCl)cc2)C(=O)NCCNC(=O)CCCCC2SCC3NC(=O)NC32)[n+](-c2c(C(C)C)cccc2C(C)C)cn1-c1c(C(C)C)cccc1C(C)C]

Treatment with 6 at 1×10⁻⁷ M resulted in a 4-fold enrichment of pre-miR-155 levels in pulled down fractions, while compound 7 at the same concentration show no enrichment. To confirm that 1 and 6 engaged the same site, cells were co-treated with 1 in dose response while 6 was held constant at 1×10⁻⁷ M. Compound 1, dose dependently reduced the enrichment of pre-miR-155 with an IC₅₀ of 1×10⁻⁷ M. Thus, compound 1 and 6 bind the same site in pre-miR-155.

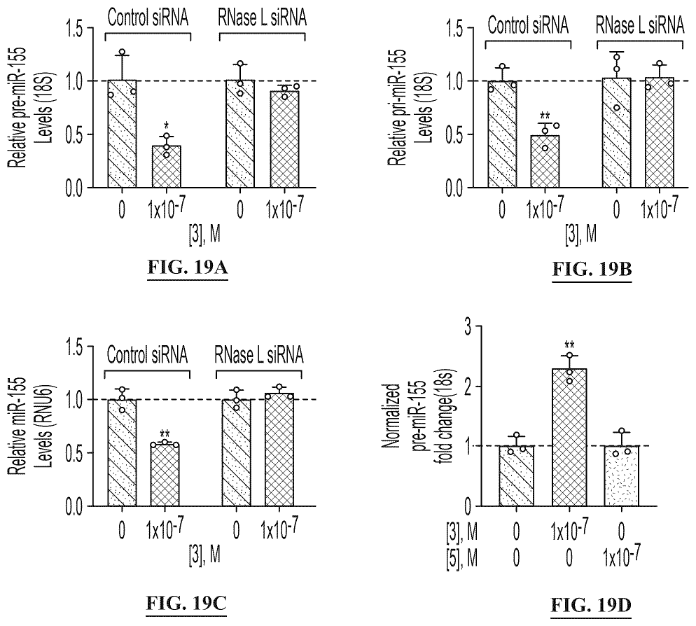

Compound 3's (Formula I) mode of action to reduce miR-155 levels was assessed. To do this, MDA-MB-231 cells were transfected with an anti-RNase L-siRNA (40 nM), which reduced RNase L levels by 85%.³¹ Then, the cells were treated with 3 at 1×10⁻⁷ M and the levels of miR-155 were measured. Knockdown of RNase L ablated 3's ability to degrade pre-miR-155 and pri-miR-155 (FIGS. 19A-19C). To further substantiate that 3's activity is a direct result of RNase L cleavage of pre-miR-155, immunoprecipitation studies of RNase L were conducted on cells treated with 3 or 5 (inactive RNase recruiter module). RT-qPCR analysis of the RNase L pulldown fractions from cells treated with 3 showed a 2.3-fold enrichment in pre-miR-155 levels, in contrast to cells treated with inactive RIBOTAC recruiter 5 which showed no enrichment (FIG. 19D). Taken together with the above Chem-CLIP studies, these data show that the activity of compound 3 is due to direct engagement of 3 with pre-miR-155 and by the recruitment of RNase L to pre-miR-155 to trigger its degradation.

### RIBOTAC Compound 3 (Formula I) Selectively Upregulates miR-155 Associated Proteins Proteome Wide and Inhibits an Oncogenic Migratory Phenotype

Having demonstrated that compound 3 is able to inhibit miR-155 levels in MDA-MB-231 cells, the effects of compound 3 (Formula I) on the proteome were studied. It has been previously reported that in triple negative breast cancer, miR-155 directly controls migration¹⁷ and angiogenesis³⁸ via the proteins suppressor of cytokine signaling 1 (SOCS1) and von Hippel-Lindau (VHL) respectively. The focus of this experimentation was upon the effect of 3 (Formula I) on MDA-MB-231's migratory phenotype.

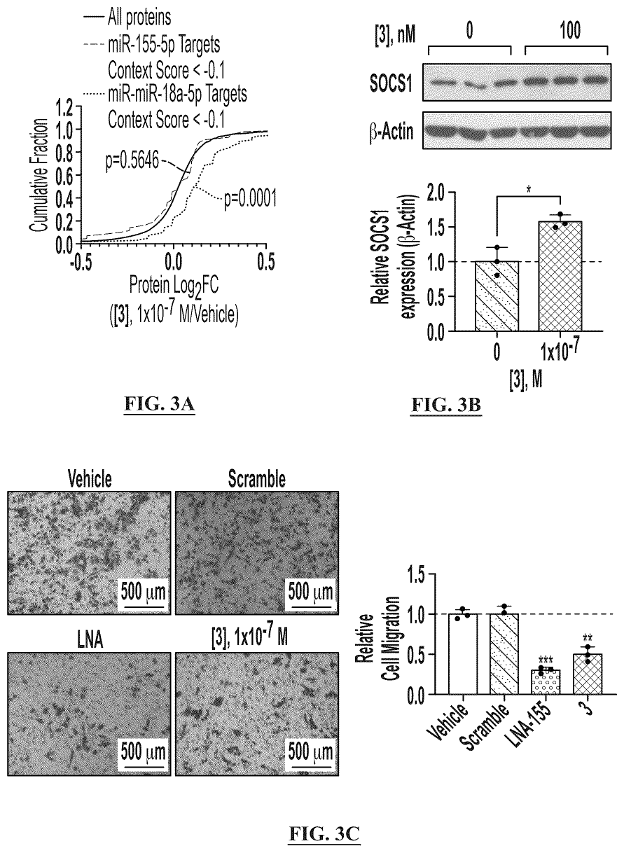

To assess compound 3's ability to modulate miR-155 associated proteins, a global proteomics analysis was conducted on cells treated with 3 at 1×10⁻⁷ M. The analysis was able to detect 3,158 proteins, of which 98 were direct targets of miR-155 according to TargetScan³⁹. These 98 proteins exhibited significant upregulation compared to all detected proteins, with a p=0.0001 (FIG. 3A). To see if this effect was specific, the effect on an isoform of equal expression to miR-155, miR-18a was assessed. The multiple miRNA study discussed above, showed that miR-18a was unaffected by compound 3, therefore, modulation of miR-18a's downstream targets is not expected. Indeed, no significant change in miR-18a associated proteins was observed (p=0.5646) (FIG. 3A).

The downstream target of miR-155, SOCS1, is reported to control the migration of MDA-MB-231 cells¹⁷. Western blotting for SOCS1 revealed that it is indeed expressed. Upon treatment with 3, SOCS1 is upregulated 1.6-fold (FIG. 3B) confirming inhibition of miR-155. The migratory capability of MDA-MB-231 cells were then measured with and without treatment of 3, an LNA oligonucleotide targeting miR-155 and a scrambled control LNA. These studies show that compound 3 at 1×10⁻⁷ M inhibited cellular migration by 50%, while the LNA-155 control inhibited migration by 70%, and the scrambled LNA control had no effect (FIG. 3C).

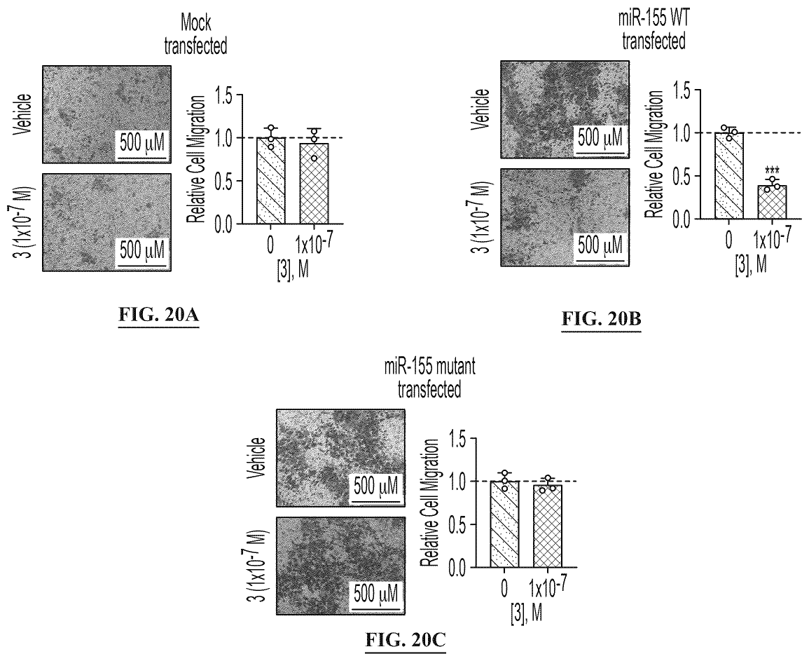

Lastly, the dependence of migration on miR-155 and the activity of 3 on the presence of the 5′GAU/3′C_A bulge was assessed. MCF-10a cells, a model of healthy breast epithelium, were transfected with wild type pre-miR-155 and mutant pre-miR-155, where the 5′GAU/3′C_A bulge is mutated to an AU base pair. Mock transfected cells showed no significant migration, nor was there an effect on these mock transfected cells with compound 3, as expected. When transfected with wild-type, and mutant pre-miR-155, however, the cellular migration of MCF-10a cells increased (FIGS. 20A and 20B), indicating that both wild type and mutant pre-miR-155 are properly processed. Treatment with 3 reduced migration of MCF-10a cells transfected with WT pre-miR-155 by 60%, in contrast to mutant miR-155 expressing cells in which the compound's binding site has been ablated, the migration of which was unaffected by 3 treatment. Taken together, these studies show that 3's ability to inhibit cellular migration is a direct result of its interaction and degradation of pre-miR-155.

### Compound 3 Potently Degrades miR-155 in Human Umbilical Vein Endothelial Cells (HUVECS) to Inhibit Angiogenesis

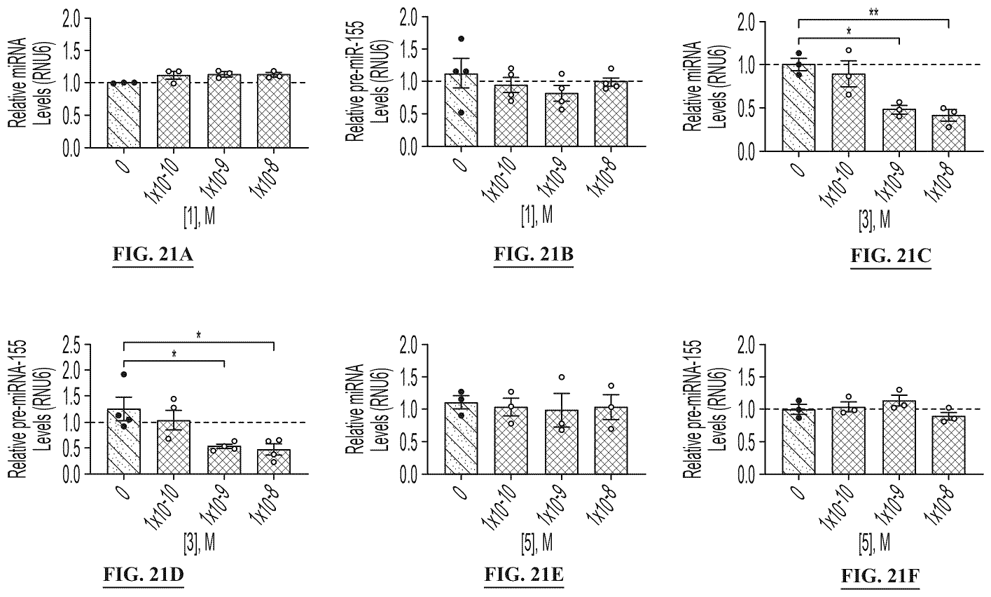

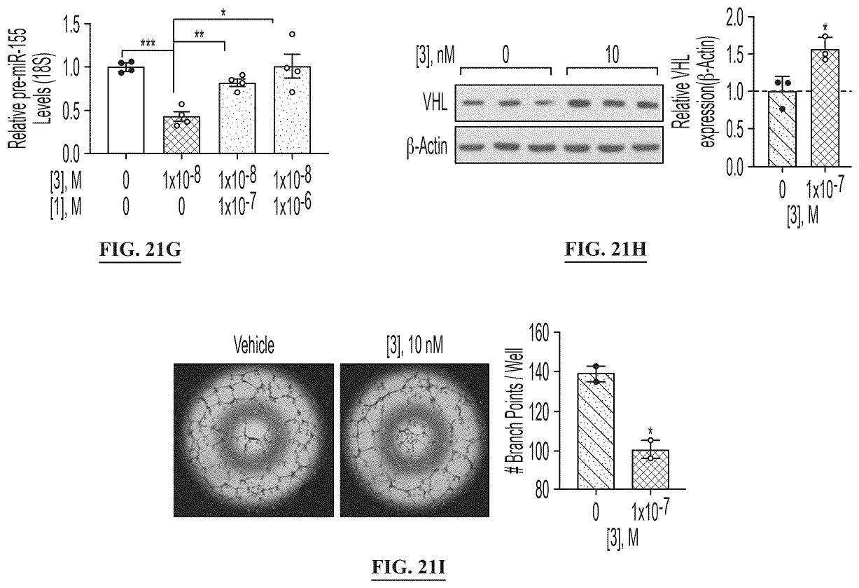

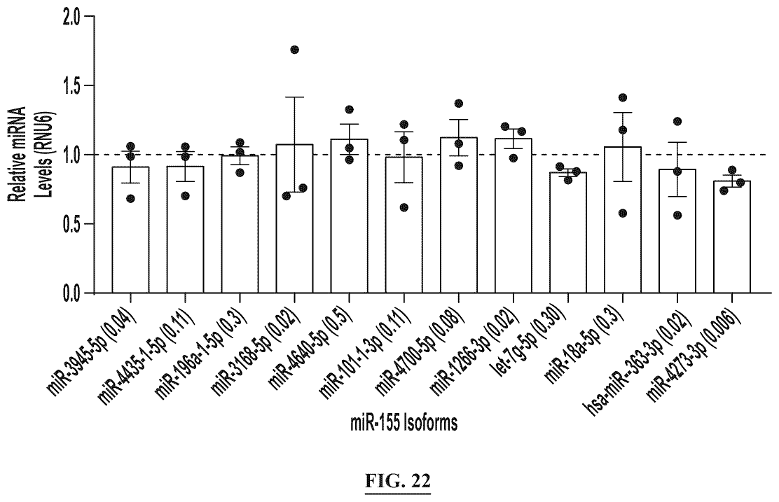

As mentioned earlier, upregulation of miR-155 is also known to promote angiogenesis in breast cancer and HUVEC models of angiogenesis. Inhibition of miR-155 by antisense oligonucleotides has been shown to decrease their angiogenic capacity via upregulation of von Hippel-Lindau.³⁸ Therefore, compound 3's ability to decrease miR-155 levels in HUVECs was studied. Treatment with simple binder compound 1 had no effect on miR-155 levels as expected (FIGS. 21A and 21B), however, treatment with RIBOTAC compound 3 (Formula I), showed dose dependent cleavage of pre-miR-155 and reduction of miR-155 levels with as little as 1×10⁻⁹ M (FIGS. 21C and 21D). Compound 5, the inactive RIBOTAC recruiter had no effect on miR-155 levels (FIGS. 21E and 21F). To assess if compound 1 and 3 compete for the same binding site in HUVECs, cells were treated with 1 in dose response and 3 at 1×10⁻⁸ M. Compound 1, dose dependently reduced cleavage by 3 at both 1×10⁻⁷ and 1×10⁻⁶ M of 1, indicating that the two molecules bind to the same site. Similar to the observation made with MDA-MB-231 cells; 3 did not affect isoforms of miR-155 even though some contain potential RIBOTAC substrates in their precursor hairpins (FIG. 22).

Lastly, compound 3's (Formula I) effect on VHL protein and angiogenesis in HUVECs was studied. Compound 3, de-repressed VHL by 1.5-fold at 1×10⁻⁷ M supporting the observation that miR-155 is being downregulated (FIG. 21H). Since upregulation of VHL should attenuate the angiogenic capacity of HUVECs, their ability to differentiate into tubule networks on Matrigel as previously described was measured.²⁴ Treatment with compound 3, caused a decrease of 29% (100 vs. 140 branch points) in tubule branching for differentiated HUVECs. Although small, this decrease does correspond with suppression of miR-155 and de-repression of VHL, which is in line with the initial study by Kong et al. who observed a 42% reduction in branching points (FIG. 21I).³⁸ Taken together compound 3 degrades miR-155 in HUVECs with low nanomolar activity and is able to attenuate their angiogenic capacity.

### Mechanism of Action and Medical Treatment

In certain embodiments, the invention is directed to methods of inhibiting, suppressing, derepressing and/or managing biolevels of the miRNA-155, pre-miRNA-155, and/or the corresponding pri-miR-155 and/or any mixture thereof as well as these RNA entities present in oncologic or inflammatory cell lines and in animals and humans having such oncologic or inflammatory cells. The Compound 3 (Formula I) as an embodiment of the invention for use in the methods disclosed herein bind to and cleave the above identified RNA entities as well in the above identified cell lines, animals and humans.

Embodiments of the Compounds applied in methods of the invention and their pharmaceutical compositions are capable of acting as “inhibitors”, suppressors and or modulators of the above identified miRNA entities which means that they are capable of blocking, suppressing or reducing the expression of the miRNA entities. An inhibitor can act with competitive, uncompetitive, or noncompetitive inhibition. An inhibitor can bind reversibly or irreversibly.

The compounds useful for methods of the invention and their pharmaceutical compositions function as therapeutic agents in that they are capable of preventing, ameliorating, modifying and/or affecting a disorder or condition. The characterization of such compounds as therapeutic agents means that, in a statistical sample, the compounds reduce the occurrence of the disorder or condition in the treated sample relative to an untreated control sample or delays the onset or reduces the severity of one or more symptoms of the disorder or condition relative to the untreated control sample.

The ability to prevent, ameliorate, modify and/or affect in relation to a condition, such as a local recurrence (e.g., pain), a disease known an oncologic disease such as but not limited to breast cancer and/or prostate cancer or any other neoplastic and/or oncologic disease or condition, especially having etiology similar to breast and/or prostate cancer may be accomplished according to the embodiments of the methods of the invention and includes administration of a composition as described above which reduces, or delays or inhibits or retards the oncologic medical condition in a subject relative to a subject which does not receive the composition.

The compounds of the invention and their pharmaceutical compositions are capable of functioning prophylactically and/or therapeutically and include administration to the host/patient of one or more of the subject compositions. If it is administered prior to clinical manifestation of the unwanted condition (e.g., disease or other unwanted state of the host animal/patient) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The compounds of the invention and their pharmaceutical compositions are capable of prophylactic and/or therapeutic treatments. If a compound or pharmaceutical composition is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof). As used herein, the term “treating” or “treatment” includes reversing, reducing, or arresting the symptoms, clinical signs, and underlying pathology of a condition in manner to improve or stabilize a subject's condition.

The compounds of the invention and their pharmaceutical compositions can be administered in “therapeutically effective amounts” with respect to the subject method of treatment. The therapeutically effective amount is an amount of the compound(s) in a pharmaceutical composition which, when administered as part of a desired dosage regimen (to a mammal, preferably a human) alleviates a symptom, ameliorates a condition, or slows the onset of disease conditions according to clinically acceptable standards for the disorder or condition to be treated, e.g., at a reasonable benefit/risk ratio applicable to any medical treatment.

### Administration

Compounds of the invention and their pharmaceutical compositions prepared as described herein can be administered according to the methods described herein through use of various forms, depending on the disorder to be treated and the age, condition, and body weight of the patient, as is well known in the art. As is consistent, recommended and required by medical authorities and the governmental registration authority for pharmaceuticals, administration is ultimately provided under the guidance and prescription of an attending physician whose wisdom, experience and knowledge control patient treatment.

For example, where the compounds are to be administered orally, they may be formulated as tablets, capsules, granules, powders, or syrups; or for parenteral administration, they may be formulated as injections (intravenous, intramuscular, or subcutaneous), drop infusion preparations, or suppositories. For application by the ophthalmic mucous membrane route or other similar transmucosal route, they may be formulated as drops or ointments.

These formulations for administration orally or by a transmucosal route can be prepared by conventional means, and if desired, the active ingredient may be mixed with any conventional additive or excipient, such as a binder, a disintegrating agent, a lubricant, a corrigent, a solubilizing agent, a suspension aid, an emulsifying agent, a coating agent, a cyclodextrin, and/or a buffer. Although the dosage will vary depending on the symptoms, age and body weight of the patient, the gender of the patient, the nature and severity of the disorder to be treated or prevented, the route of administration and the form of the drug, in general, a daily dosage of from 0.0001 to 2000 mg, preferably 0.001 to 1000 mg, more preferably 0.001 to 500 mg, especially more preferably 0.001 to 250 mg, most preferably 0.001 to 150 mg of the compound is recommended for an adult human patient, and this may be administered in a single dose or in divided doses. Alternatively, a daily dose can be given according to body weight such as 1 nanogram/kg (ng/kg) to 200 mg/kg, preferably 10 ng/kg to 100 mg/kg, more preferably 10 ng/kg to 10 mg/kg, most preferably 10 ng/kg to 1 mg/kg. The amount of active ingredient which can be combined with a carrier material to produce a single dosage form will generally be that amount of the compound which produces a therapeutic effect.

The precise time of administration and/or amount of the composition that will yield the most effective results in terms of efficacy of treatment in a given patient will depend upon the activity, pharmacokinetics, and bioavailability of a particular compound, physiological condition of the patient (including age, sex, disease type and stage, general physical condition, responsiveness to a given dosage, and type of medication), route of administration, etc. However, the above guidelines can be used as the basis for fine-tuning the treatment, e.g., determining the optimum time and/or amount of administration, which will require no more than routine experimentation consisting of monitoring the subject and adjusting the dosage and/or timing.

The phrase “pharmaceutically acceptable” is employed herein to refer to those excipients, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

### Pharmaceutical Compositions Incorporating Compound 3 AKA Formula I

The pharmaceutical compositions of the invention incorporate embodiments of Compound 3 also known as (aka) Formula I useful for methods of the invention and a pharmaceutically acceptable carrier. The compositions and their pharmaceutical compositions can be administered orally, topically, parenterally, by inhalation or spray or rectally in dosage unit formulations. The term parenteral is described in detail below. The nature of the pharmaceutical carrier and the dose of these Compounds depend upon the route of administration chosen, the effective dose for such a route and the wisdom and experience of the attending physician.

A “pharmaceutically acceptable carrier” is a pharmaceutically acceptable material, composition, or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose, and sucrose; (2) starches, such as corn starch, potato starch, and substituted or unsubstituted (3-cyclodextrin; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers, and lubricants, such as sodium lauryl sulfate and magnesium stearate, as well as coloring agents, release agents, coating agents, sweetening, flavoring, and perfuming agents, preservatives and antioxidants can also be present in the compositions. Examples of pharmaceutically acceptable antioxidants include: (1) water soluble antioxidants, such as ascorbic acid, cysteine hydrochloride, sodium bisulfate, sodium metabisulfite, sodium sulfite, and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert matrix, such as gelatin and glycerin, or sucrose and acacia) and/or as mouthwashes, and the like, each containing a predetermined amount of a compound of the invention as an active ingredient. A composition may also be administered as a bolus, electuary, or paste.

In solid dosage form for oral administration (capsules, tablets, pills, dragees, powders, granules, and the like), a compound of the invention is mixed with one or more pharmaceutically acceptable carriers, such as sodium citrate or dicalcium phosphate, and/or any of the following:
(1) fillers or extenders, such as starches, cyclodextrins, lactose, sucrose, glucose, mannitol, and/or silicic acid;
(2) binders, such as, for example, carboxymethylcellulose, alginates, gelatin, polyvinyl pyrrolidone, sucrose, and/or acacia;
(3) humectants, such as glycerol;
(4) disintegrating agents, such as agar-agar, calcium carbonate, potato or tapioca starch, alginic acid, certain silicates, and sodium carbonate;
(5) solution retarding agents, such as paraffin;
(6) absorption accelerators, such as quaternary ammonium compounds;
(7) wetting agents, such as, for example, acetyl alcohol and glycerol monostearate; (8) absorbents, such as kaolin and bentonite clay;
(9) lubricants, such a talc, calcium stearate, magnesium stearate, solid polyethylene glycols, sodium lauryl sulfate, and mixtures thereof; and
(10) coloring agents. In the case of capsules, tablets, and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols, and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered inhibitor(s) moistened with an inert liquid diluent.

Tablets, and other solid dosage forms, such as dragees, capsules, pills, and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes, and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner.

Examples of embedding compositions which can be used include polymeric substances and waxes. A compound of the invention can also be in micro-encapsulated form, if appropriate, with one or more of the above-described excipients.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents, and emulsifiers such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols, and fatty acid esters of sorbitan, and mixtures thereof.

Besides inert diluents, the oral compositions can also include adjuvants such as wetting agents, emulsifying and suspending agents, sweetening, flavoring, coloring, perfuming, and preservative agents.

Suspensions, in addition to the active inhibitor(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more inhibitor(s) with one or more suitable nonirritating excipients or carriers comprising, for example, cocoa butter, polyethylene glycol, a suppository wax or a salicylate, which is solid at room temperature, but liquid at body temperature and, therefore, will melt in the rectum or vaginal cavity and release the active agent.

Formulations which are suitable for vaginal administration also include pessaries, tampons, creams, gels, pastes, foams, or spray formulations containing such carriers as are known in the art to be appropriate.

Dosage forms for the topical or transdermal administration of an inhibitor(s) include powders, sprays, ointments, pastes, creams, lotions, gels, solutions, patches, and inhalants. The active component may be mixed under sterile conditions with a pharmaceutically acceptable carrier, and with any preservatives, buffers, or propellants which may be required.

The ointments, pastes, creams, and gels may contain, in addition to a compound of the invention, excipients, such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to a compound of the invention, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

A compound useful for application of methods of the invention can be alternatively administered by aerosol. This is accomplished by preparing an aqueous aerosol, liposomal preparation, or solid particles containing the composition. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of a compound of the invention together with conventional pharmaceutically acceptable carriers and stabilizers. The carriers and stabilizers vary with the requirements of the particular composition, but typically include nonionic surfactants (Tweens, Pluronics, sorbitan esters, lecithin, Cremophors), pharmaceutically acceptable co-solvents such as polyethylene glycol, innocuous proteins like serum albumin, oleic acid, amino acids such as glycine, buffers, salts, sugars, or sugar alcohols. Aerosols generally are prepared from isotonic solutions.

Transdermal patches have the added advantage of providing controlled delivery of a compound of the invention to the body. Such dosage forms can be made by dissolving or dispersing the agent in the proper medium. Absorption enhancers can also be used to increase the flux of the inhibitor(s) across the skin. The rate of such flux can be controlled by either providing a rate controlling membrane or dispersing the inhibitor(s) in a polymer matrix or gel.

Pharmaceutical compositions of this invention suitable for parenteral administration comprise one or more compounds of the invention in combination with one or more pharmaceutically acceptable sterile aqueous or nonaqueous solutions, dispersions, suspensions or emulsions, or sterile powders which may be reconstituted into sterile injectable solutions or dispersions just prior to isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include tonicity-adjusting agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a compound useful for practice of methods of the invention, it is desirable to slow the absorption of the compound from subcutaneous or intramuscular injection. For example, delayed absorption of a parenterally administered drug form is accomplished by dissolving or suspending the drug in an oil vehicle.

Injectable depot forms are made by forming microencapsule matrices of inhibitor(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

The pharmaceutical compositions may be given orally, parenterally, topically, or rectally. They are, of course, given by forms suitable for each administration route. For example, they are administered in tablets or capsule form, by injection, inhalation, eye lotion, ointment, suppository, infusion; topically by lotion or ointment; and rectally by suppositories. Oral administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection, and infusion.

The pharmaceutical compositions of the invention may be “systemically administered” “administered systemically,” “peripherally administered” and “administered peripherally” meaning the administration of a ligand, drug, or other material other than directly into the central nervous system, such that it enters the patient's system and thus, is subject to metabolism and other like processes, for example, subcutaneous administration.

The compound(s) useful for application of the methods of the invention may be administered to humans and other animals for therapy by any suitable route of administration, including orally, nasally, as by, for example, a spray, rectally, intravaginally, parenterally, intracisternally, and topically, as by powders, ointments or drops, including buccally and sublingually.

Regardless of the route of administration selected, the compound(s) useful for application of methods of the invention, which may be used in a suitable hydrated form, and/or the pharmaceutical compositions of the present invention, are formulated into pharmaceutically acceptable dosage forms by conventional methods known to those of skill in the art.

Actual dosage levels of the compound(s) useful for application of methods of the invention in the pharmaceutical compositions of this invention may be varied so as to obtain an amount of the active ingredient which is effective to achieve the desired therapeutic response for a particular patient, composition, and mode of administration, without being toxic to the patient.

The concentration of a compound useful for application of methods of the invention in a pharmaceutically acceptable mixture will vary depending on several factors, including the dosage of the compound to be administered, the pharmacokinetic characteristics of the compound(s) employed, and the route of administration.

In general, the compositions useful for application of methods of this invention may be provided in an aqueous solution containing about 0.1-10% w/v of a compound disclosed herein, among other substances, for parenteral administration. Typical dose ranges are those given above and may preferably be from about 0.001 to about 500 mg/kg of body weight per day, given in 1-4 divided doses. Each divided dose may contain the same or different compounds of the invention. The dosage will be an effective amount depending on several factors including the overall health of a patient, and the formulation and route of administration of the selected compound(s).

### REFERENCES

1 Frazier, K. S. Antisense oligonucleotide therapies: the promise and the challenges from a toxicologic pathologist's perspective. Toxicol. Pathol. 43, 78-89, doi:10.1177/0192623314551840 (2015).
2 Lenartowicz, E. et al. Antisense oligonucleotides targeting influenza A segment 8 henomic RNA inhibit viral replication. Nucleic Acid Ther. 26, 277-285, doi:10.1089/nat.2016.0619 (2016).
3 Mandal, M. & Breaker, R. R. Gene regulation by riboswitches. Nat Rev Mol Cell Biol 5, 451-463, doi:10.1038/nrm1403 (2004).
4 Winkler, W., Nahvi, A. & Breaker, R. R. Thiamine derivatives bind messenger RNAs directly to regulate bacterial gene expression. Nature 419, 952-956, doi:10.1038/nature01145 (2002).
Rizvi, N. F. et al. Targeting RNA with Small Molecules: Identification of Selective, RNA-Binding Small Molecules Occupying Drug-Like Chemical Space. SLAS discovery: advancing life sciences R & D 25, 384-396, doi:10.1177/2472555219885373 (2020).
6 Costales, M. G., Childs-Disney, J. L., Haniff, H. S. & Disney, M. D. How we think about targeting RNA with small molecules. J. Med. Chem., Article ASAP, doi:10.1021/acs.jmedchem.9b01927 (2020).
7 Karageorgis, G., Foley, D. J., Laraia, L. & Waldmann, H. Principle and design of pseudo-natural products. Nature Chemistry 12, 227-235, doi:10.1038/s41557-019-0411-x (2020).
8 Wicks, S. L. & Hargrove, A. E. Fluorescent indicator displacement assays to identify and characterize small molecule interactions with RNA. Methods 167, 3-14, doi:10.1016/j.ymeth.2019.04.018 (2019).
9 Velagapudi, S. P. & Disney, M. D. Two-dimensional combinatorial screening enables the bottom-up design of a microRNA-10b inhibitor. Chem Commun (Camb) 50, 3027-3029, doi:10.1039/c3cc00173c (2014).
Velagapudi, S. P. et al. Defining RNA-Small Molecule Affinity Landscapes Enables Design of a Small Molecule Inhibitor of an Oncogenic Noncoding RNA. ACS Cent. Sci. 3, 205-216, doi:10.1021/acscentsci.7b00009 (2017).
11 Disney, M. D. et al. Inforna 2.0: A Platform for the Sequence-Based Design of Small Molecules Targeting Structured RNAs. ACS Chem Biol 11, 1720-1728, doi:10.1021/acschembio.6b00001 (2016).
12 Morgan, B. S., Forte, J. E., Culver, R. N., Zhang, Y. & Hargrove, A. E. Discovery of Key Physicochemical, Structural, and Spatial Properties of RNA-Targeted Bioactive Ligands. Angew. Chem. Int. Ed. Engl. 56, 13498-13502, doi:10.1002/anie.201707641 (2017).
13 Bajusz, D., Ricz, A. & H6berger, K. Why is Tanimoto index an appropriate choice for fingerprint-based similarity calculations?Journal of Cheminformatics 7, 20, doi:10.1186/s13321-015-0069-3 (2015).
14 Nettling, M. et al. DiffLogo: a comparative visualization of sequence motifs. BMC bioinformatics 16, 387, doi:10.1186/s12859-015-0767-x (2015).
Han, Y. et al. Structure of human RNase L reveals the basis for regulated RNA decay in the IFN response. Science 343, 1244-1248, doi: 10.1126/science.1249845 (2014).
16 Huang, Z. et al. HMDD v3.0: a database for experimentally supported human microRNA-disease associations. Nucleic Acids Research 47, D1013-D1017, doi:10.1093/nar/gky1010 (2018).
17 Jiang, S. et al. MicroRNA-155 Functions as an OncomiR in Breast Cancer by Targeting the <em>Suppressor of Cytokine Signaling 1</em>Gene. Cancer Research 70, 3119-3127, doi:10.1158/0008-5472.Can-09-4250 (2010).
18 Guedes, J. R. et al. Early miR-155 upregulation contributes to neuroinflammation in Alzheimer's disease triple transgenic mouse model. Hum Mol Genet 23, 6286-6301, doi:10.1093/hmg/ddu348 (2014).
19 Velagapudi, S. P., Gallo, S. M. & Disney, M. D. Sequence-based design of bioactive small molecules that target precursor microRNAs. Nat. Chem. Biol. 10, 291-297, doi:10.1038/Nchembio.1452 (2014).
Childs-Disney, J. L. et al. A Massively Parallel Selection of Small Molecule-RNA Motif Binding Partners Informs Design of an Antiviral from Sequence. Chem 4, 2384-2404, doi:10.1016/j.chempr.2018.08.003 (2018).
21 Yesselman, J. D. et al. Sequence-dependent RNA helix conformational preferences predictably impact tertiary structure formation. Proc. Natl. Acad. Sci. U.S.A. 116, 16847-16855, doi:10.1073/pnas.1901530116 (2019).
22 Haniff, H. S., Graves, A. & Disney, M. D. Selective Small Molecule Recognition of RNA Base Pairs. ACS Comb Sci 20,482-491, doi:10.1021/acscombsci.8b00049 (2018).
23 Haniff, H. S. et al. Small molecules exploiting structural differences within microRNA-200 precursors family members reverse a type 2 diabetes phenotype. bioRxiv, 2020.2006.2027.175281, doi:10.1101/2020.06.27.175281 (2020).
24 Hafeez, H. et al. Design of a Small Molecule That Stimulates VEGFA Informed from an Expanded Encyclopedia of RNA Fold-Small Molecule Interactions. (2020).
Costales, M. G. et al. A Designed small molecule inhibitor of a non-coding RNA sensitizes HER2 negative cancers to herceptin. J. Am. Chem. Soc. 141, 2960-2974, doi:10.1021/jacs.8b10558 (2019).
26 Costales, M. G., Suresh, B., Vishnu, K. & Disney, M. D. Targeted Degradation of a Hypoxia-Associated Non-coding RNA Enhances the Selectivity of a Small Molecule Interacting with RNA. Cell Chem Biol 26, 1180-1186 e1185, doi:10.1016/j.chembiol.2019.04.008 (2019).
27 Costales, M. G. et al. Small molecule inhibition of microRNA-210 reprograms an oncogenic hypoxic circuit. J. Am. Chem. Soc. 139, 3446-3455, doi:10.1021/jacs.6b11273 (2017).
28 Liu, X. et al. Targeted degradation of the oncogenic microRNA 17-92 cluster by structure-targeting ligands. J. Am. Chem. Soc. 142, 6970-6982, doi:10.1021/jacs.9bl3159 (2020).
29 Angelbello, A. J. & Disney, M. D. Bleomycin Can Cleave an Oncogenic Noncoding RNA. ChemBioChem 19, 43-47, doi:10.1002/cbic.201700581 (2018).
30 Costales, M. G. et al. Small-molecule targeted recruitment of a nuclease to cleave an oncogenic RNA in a mouse model of metastatic cancer. Proc. Nat'l. Acad. Sci. U.S.A. 117, 2406-2411, doi:10.1073/pnas.1914286117 (2020).
31 Costales, M. G., Matsumoto, Y., Velagapudi, S. P. & Disney, M. D. Small molecule targeted recruitment of a nuclease to RNA. J. Am. Chem. Soc. 140, 6741-6744, doi:10.1021/jacs.8b01233 (2018).
32 Angelbello, A. J., DeFeo, M. E., Glinkerman, C. M., Boger, D. L. & Disney, M. D. Precise Targeted Cleavage of a r(CUG) Repeat Expansion in Cells by Using a Small-Molecule-Deglycobleomycin Conjugate. ACS Chem Biol 15, 849-855, doi:10.1021/acschembio.0c00036 (2020).
33 Angelbello, A. J. et al. Precise small-molecule cleavage of an r(CUG) repeat expansion in a myotonic dystrophy mouse model. Proc. Nat'l. Acad. Sci. U.S.A. 116, 7799-7804, doi:10.1073/pnas.1901484116 (2019).
34 Dacic, S., Kelly, L., Shuai, Y. & Nikiforova, M. N. miRNA expression profiling of lung adenocarcinomas: correlation with mutational status. Mod Pathol 23, 1577-1582, doi:10.1038/modpathol.2010.152 (2010).
35 Al-Ahmadi, W., Al-Haj, L., Al-Mohanna, F. A., Silverman, R. H. & Khabar, K. S. RNase L downmodulation of the RNA-binding protein, HuR, and cellular growth. Oncogene 28, 1782-1791, doi:10.1038/onc.2009.16 (2009).
36 Marzi, M. J. et al. Degradation dynamics of microRNAs revealed by a novel pulse-chase approach. Genome Res 26, 554-565, doi:10.1101/gr.198788.115 (2016).
37 Gantier, M. P. et al. Analysis of microRNA turnover in mammalian cells following Dicer1 ablation. Nucleic Acids Res 39, 5692-5703, doi:10.1093/nar/gkrl48 (2011).
38 Kong, W. et al. Upregulation of miRNA-155 promotes tumour angiogenesis by targeting VHL and is associated with poor prognosis and triple-negative breast cancer. Oncogene 33, 679-689, doi:10.1038/onc.2012.636 (2014).
39 Agarwal, V., Bell, G. W., Nam, J.-W. & Bartel, D. P. Predicting effective microRNA target sites in mammalian mRNAs. eLife 4, e05005, doi:10.7554/eLife.05005 (2015).
40 Disney, M. D. Targeting RNA with Small Molecules To Capture Opportunities at the Intersection of Chemistry, Biology, and Medicine. J. Am. Chem. Soc. 141, 6776-6790, doi:10.1021/jacs.8b13419 (2019).
41 Zhang, P. et al. Translation of the intrinsically disordered protein a-synuclein is inhibited by a small molecule targeting its structured mRNA. Proc. Nat'l. Acad. Sci. U.S.A. 117, 1457-1467, doi:10.1073/pnas.1905057117 (2020).
42 Haniff, H. S., Knerr, L., Chen, J. L., Disney, M. D. & Lightfoot, H. L. Target-directed approaches for screening small molecules against RNA targets. SLAS Discov., 2472555220922802, doi:10.1177/2472555220922802 (2020).
43 Wang, Z. F. et al. The hairpin form of r(G4C2)(exp) in c9ALS/FTD is repeat-associated non-ATG translated and a target for bioactive small molecules. Cell Chem. Biol. 26, 179-190 e112, doi:10.1016/j.chembiol.2018.10.018 (2019).
44 Velagapudi, S. P. et al. Design of a small molecule against an oncogenic noncoding RNA. Proc. Natl. Acad. Sci. U.S.A. 113, 5898-5903, doi:10.1073/pnas.1523975113 (2016).

### Methods

General. All DNA templates and primers were purchased from Integrated DNA Technologies (IDT) and used directly. Chemically synthesized RNA and oligonucleotide competitors were obtained from Dharmacon and deprotected by incubation with deprotection buffer per the manufacturers protocol. After deprotection the RNAs were desalted using a PD-10 sephadex column (GE Healthcare) according to the manufacturers protocol. Briefly the columns were equilibrated with 10 column volumes of nano pure water. The RNA was then loaded and eluted in 3 column volumes of water collecting 1 mL fractions. All autoradiography was obtained on a Typhoon FLA9500 variable mode imager (GE Healthcare) and the band were quantified using Quantity One (Bio-Rad) software. All oligonucleotides were quantified by UV-Vis at 90° C. using their absorption at 260 nM. For RNA sequencing, cDNA samples were quantified on an Agilent Technologies 2100 Bioanalyzer (Model #: G1939A) and on a Qbit 2.0 (Invitrogen) fluorimeter. Sequencing was done on an Ion Proton sequencer (Life Technologies) with >200-fold coverage/base. Cells were grown in RPMI 1640 (Corning) supplemented with 10% (v/v) FBS, and 1× antibiotic/antimycotic (Corning). All cells were grown at 37° C. in 5% CO₂.

All RT-qPCR experiments were performed on an Applied Biosystems QS5 384 well PCR system. and gene expressions were tested by using Power Sybr Green Mater mix (Life Technologies). Radioactive 32P-γ-ATP was purchased from Perkin Elmer and used directly to label RNA. Radiolabeled gels were scanned on a Typhoon FLA9500 (General Electric) was used to scan radiolabeled gels and images were analyzed and quantified by Quantity One image analysis software (Bio-Rad).

HPLC purification was performed with Waters 1525 Binary HPLC Pump equipped with a Waters 2487 Dual Absorbance Detector system. A reverse phase Atlantis®Prep T3 C18 5 μM column used for purification. The gradient used for purification is from 100% of H₂O (containing 0.1% TFA) to 100% MeOH(containing 0.1% TFA) in 60 min. Purity of the products were evaluated with a analytical HPLC equipped with a reverse phase column-Waters Symmetry C18 5 μm 4.6×150 mm column with a flow rate of 1 mL/min from 100% of H₂O(containing 0.1% TFA) to 100% MeOH(containing 0.1% TFA) in 60 min. The detected absorbance was at 220 nm and 254 nm. Mass spectra were obtained on a 4800 plus MALDI TOF/TOF analyzer. All NMR spectra were obtained by using a Bruker 400 UltraShield™. The chemical shifts listed are shown in ppm relative to residual solvents for ¹H and ¹³C as internal standards. Coupling constants(J) are described in hertz.

Chemicals were purchased from the suppliers without further purification. Chemicals used in this study are from the following suppliers: HATU and trifluoroacetic acid from Oakwood Chemical; 2, 6-diisoproplaniline, n-BuLi and hydrogen bromide from Alfa Aesar; diacetyl from TCI; ethyl bromoacetate and paraformaldehyde from Acros Organics; and anhydrous dimethyl sulfoxide and anhydrous N,N-dimethylformamide from EMD.

### Screening of COMAS Library by TO-Pro-1

Validation. The RNA library was transcribed as described previously.⁸ TO-Pro-1 was obtained from Life Technologies (Thermo Fisher T3602) and used directly as a 1 mM DMSO stock solution. RNA solutions were prepared in DNase RNase free water supplemented with 1×DNA buffer (8.0 mM Na₂PO₄, 185 mM NaCl, pH=7.0) and 3×3 ILL at 100 or 200 nM. The RNA was annealed at 75° C. for 30 min followed by slow cooling to room temperature on the benchtop. After cooling, TO-Pro-1 was added to a final concentration of 100 or 200 nM, equimolar to the RNA and allowed to equilibrate for 30 min at room temperature. During equilibration, the Multidrop Combi nL® (ThermoFisher) was washed with nanopure water per the manufacturers protocol. The solution containing the TO-Pro-1 and RNA mixture were then plated in 1536 format (Greiner 782076) at 5 μL/well in duplicate. After plating, the plates were spun down and read on a Tecan Safire* (Tecan) plate reader (Ex. 485±5 nm; Em. 520±1 nm) with a gain of 225 optimized on empty wells. After reading, compounds were dosed using a 1536 well pin transfer tool by the TSRI HTS core, transferring 50 nL of Hoechst 33258 (0, 1,10 and 100 μM final concentration) or DMSO into each well. Dosed plates were then sealed and incubated for 1 h at room temperature followed by readout of the TO-Pro-1 signal using the aforementioned settings. Percent displacement was calculated using equation (1) below.

$$( \frac{{F}_{B} - {F}_{A}}{{F}_{B}} ) * 100 = \% Displacement \qquad ( 1 )$$

where F_{B} is the fluorescence before compound addition and FA is the fluorescence after compound addition.

To determine this assay's amenability to HTS screening in this format, a Z-factor was calculated according to equation (2):

$$Estimated Z Factor = 1 - \frac{3 ( {σ}_{p} + {σ}_{n} )}{\vert {μ}_{p} - {μ}_{n} \vert} \qquad ( 2 )$$

where σₚ and σₙ are the standard deviations of the percent changes of the positive and negative controls respectively and μₚ and μₙ are the mean percent changes for the positive and negative control respectively.

Validation was replicated on the COMAS screening platform which uses a Thermo Fisher F5 automation system integrating plate hotels for incubation, an Echo520 acoustic dispenser for compounds (Labcyte Inc.), Multidrop Combi nL (Thermo Fisher) dispensers for plating solutions and an Envision plate reader (PerkinElmer) for reading out emission. A signal to noise ratio of 6 and Z-factor of 0.67 were obtained readily.

COMAS Screening. The 3×3 ILL was prepared as described above. A control RNA lacking the randomized region of the 3×3 ILL was used as a control counter screen and transcribed as described previously. Its sequence can be found in Table 4. The RNA solutions were made as described earlier. Using the Multidrop Combi dispensers, 5 μL of the Dye/RNA mixture were added to each well in a 1536 well plate Greiner (782076) with buffer containing TO-Pro-1 only being added to the control wells. The plates were then read out on a PerkinElmer EnVision® plate reader (Ex/Em: 480/535). After reading, 5 nL of compound was added using an Echo 520 acoustic dispenser (Labcyte Inc.) and the plates incubated at room temperature in the plate hotels. All data were normalized to DMSO controls and then % displacement calculated as described above. This procedure was repeated for the base paired control RNA as well. The signal was triaged to compounds that had reduced the signal below 30% and then these were cherry picked for dose responsive behavior. Dose responsive behavior was done on 330 compounds with a 3-fold dilution across eight steps to a final concentration from 30 μM to 0.001 μM. Curves were fit to give ICso's using the Quatro Workflow software (Quattro-Research GmbH). Dose dependency was done in triplicate. These 330 compounds were then screened by 2DCS to annotate their RNA binding landscapes.

Buffers used in 2DCS. These buffers and the methods for 2DCS below were described previously and are as follows.^{9, 10} 1× Folding Buffer (FB): 20 mM HEPES, pH 7.5, 150 mM NaCl, 5 mM KCl; 1× Hybridization Buffer (HB): 20 mM HEPES, pH 7.5, 150 mM NaCl, 5 mM KCl, 1 mM MgCl₂ and 40 μg/mL BSA; 10× PCR Buffer: 100 mM Tris, pH 9.0, 500 mM KCl and 1% Triton X-100.

Preparation of RNA Libraries. PCR amplification was done as previously described.¹⁰ Briefly, amplification of DNA templates by PCR was done in 1× PCR buffer supplemented with 0.33 μL of 5 mM dNTPs, 4.25 mM MgCl₂, 500 nM of reverse primer, and 500 nM of forward primer, 20 nM of DNA template and 2 μL of Taq DNA polymerase. The amplification was completed using three-step PCR as follows: 95° C. for 60 s, 50° C. for 30 s, and 72° C. for 60 s. The size of PCR products were verified by 3% agarose gel and then transcribed in vitro using a Stratagene RNAMaxx™ High Yield transcription kit following the manufacturers guidelines. To hot label the libraries. The purified RNA transcripts were phosphatased with Calf Intestinal Phosphatase (Promega) and then Kinased with T-³²P ATP using T4 polynucleotide kinase (New England Biolabs) according to the manufacturer's guidelines. The RNA was purified by 15% denaturing polyacrylamide gel electrophoresis and quantified by UV-Vis using 10,800 M⁻¹ cm⁻¹/nucleotide to estimate the extinction coefficients.

2DCS: Primary Screening. The microarrays were constructed as previously described.¹⁰ Microarrays were by coating glass plates with 25 mL of molten 1% agarose and allowing it to set for 2 h. After setting, the plates were pinned with compound using a Biomek® NX robotic pintool, pinning 100 nL of each compound. The array was dried in a fume hood overnight and then washed in 1× FB supplemented with 0.1% (v/v) Tween-20 and then Nano pure water two times each followed by air drying on the bench top. Once dried, 200 pmol of radiolabeled 3×3 ILL was added to 0.5 mL of 1× FB and folded by heating to 95° C. for 1 min and then cooling to room temperature slowly on the benchtop. Magnesium chloride was added after cooling. Once cooled the solution was brought to a volume of 2.5 mL with 1× FB. The microarrays were pre-hybridized with 1× HB for 5 min and the excess buffer was removed by touching the edge with a Kimwipe. The arrays were then Hybridized with the RNA for 30 min, spreading the solution evenly over the surface with parafilm. After hybridization, the array was washed four times with 1× HB followed by air drying and imaging by autoradiography. Hits were identified as signals with an intensity greater than 3-fold over background radiation on the array. Positive control compounds Mitoxantrone was present on all arrays.

2DCS: tRNA Counter Screen. To remove non-specific RNA binders hits identified above were subjected to competitive screening with cold yeast tRNA. The assay was carried out as previously described.¹⁰ Briefly, arrays were constructed on Inkjet Superfrost microscope slides (Fisher) by applying 2 mL of molten 1% agarose onto each slide and curing them for 2 h. Once cured, 200 nL of each compound was spotted onto each plate and the plates dried overnight as previously described. After drying, the slides were washed three times for 5 min each with nano pure water and dried under a stream of compressed air. The slides were then pre-hybridized with 1× HB for 5 min, and then incubated with 100 pmol of folded hot RNA and tRNA (1× relative to the total moles of compound spotted) in 0.4 mL of 1× FB for 30 min. The slides were then washed with 1× HB five times, air dried, and imaged to identify library specific binders.

2DCS: Competitor Oligonucleotide Screening. Microarrays were constructed and screened as described previously.¹⁰ Arrays were made as mentioned earlier on microscope slides except hits were spotted in a dose response from 10 mM to 0.625 mM, with each slide holding a maximum of eight compounds (31 nmol of compounds per slide). Competitor oligonucleotide samples were prepared as follows. Stem, Tail, and Hairpin RNA competitors, d(GC)₁₁ and d(AU)₁₁ and tRNA were all folded separately in 0.05 mL of 1× FB. Upon cooling 100 pmol of hot 3×3 ILL RNA was added and the solution brought to a final volume of 0.4 mL. All competitors are 310× relative to the total moles of compound spotted. Pre-hybridization and hybridization were carried out as described above. RNA that was bound to the compounds were excised if the signal was >3-fold above the background radiation of the array. Both the excised RNAs and the unselected library was sequenced.

Statistical analysis of selected RNAs. The HiT-StARTS statistical analysis methodology was applied to this data as previously described.¹⁰ Briefly, to identify statistically significant enrichments in bound RNAs from the 3×3 ILL a pooled population analysis was conducted to calculate a Z-score (Z_{obs}) for each sequence using equation (3) and (4) below by comparing frequency of reads in the selected library to the starting library.

$$ϕ = \frac{{n}_{1} {p}_{1} - {n}_{2} {p}_{2}}{{n}_{1} + {n}_{2}} \qquad ( 3 )$$

$${Z}_{o b s} = \frac{( {p}_{1} - {p}_{2} )}{\sqrt{ϕ ( 1 - ϕ ) ( \frac{1}{{n}_{1}} + \frac{1}{{n}_{2}} )}} \qquad ( 4 )$$

In these equations n₁ is the observed reads for all selected RNAs in the RNA-seq data; n₂ is the total reads observed for the starting library; p₁ is the proportion of the reads for a particular sequence to the total reads in the selected library; p₂ is the proportion of the reads for a particular sequence to the total reads of the starting library. We can then use Z_{obs} to rank each sequence, and therefore each RNA motif, by its affinity or fitness which is calculated according to equation (5) below described previously:¹⁰

$$Fitness = \frac{{Z}_{n} - {Z}_{obs ( 8 )}}{{Z}_{obs max} - {Z}_{obs ( 8 )}} * 100 \qquad ( 5 )$$

where Z, is the Z_{obs} for a particular sequence in the RNA-seq data; Z_{obs (8)}=8 for all non-fragment small molecule RNA binders as previously shown^{9,10}; Z_{obs max} is the Z_{obs} for the highest ranked sequences (rank 1) of the RNA-seq data.

Tissue Culture. MDA-MB-231 cells were obtained from ATCC (HTB-26) and cultured in RPMI medium with L-30 glutamine & 25 mM HEPES (Corning) supplemented with 10% FBS (sigma). MCF-10a cells were obtained from ATCC(CRL-10317) and cultured in DMEM/F12 50/50 with glutamine and 15 mM HEPES(Corning) containing 20% FBS(Sigma), 1×Antibiotic-Antimycotic(Corning), 20 ng/mL of human epidermal growth factor(Pepro Tech Inc.), 100 μg/mL of insulin and 0.5 mg/mL of hydrocortisone (Pfizer &Bauer). HUVECs were cultured in EGM (Lonza) made using the EGM-2 bullet kit (Lonza) per the manufacturers protocol.

PCR amplification and transcription of DNA templates. PCR amplification of DNA templates of pre-miR-155 and mutants were performed using the pre-miR-155 forward primer containing T7 RNA polymerase promoter and a reverse primer common to both the pre-miR-155 Wild type and Mutant templates. The sequence of Oligonucleotides used in this study can be found in Table 4. PCR amplification was carried out in 350 μL of 1× PCR buffer (10 mM Tris-HCl, pH 9.0, 50 mM KCl, and 0.1% (v/v) Triton X-100), 0.33 mM dNTPs, 4.25 mM MgCl₂, 2 μM of each primer(100 μM), and 1.7 μL of Taq DNA polymerase. Thermocycling was done for 35 cycles at 95° C. for 30 s, 55° C. for 30 s, and 72° C. for 60 s. PCR products were confirmed by a 2.5% Agarose Gel stained with ethidium bromide before in vitro transcription. In vitro RNA transcription for the target RNAs were carried out using house made reagents as described previously.¹¹

Tanimoto scoring analysis. Small molecule structures were analyzed for chemical similarity to the Inforna⁸ and R-BIND¹² databases of RNA-small molecule binders.

Using instant JChem (ChemAxon), an overlap analysis was done using Chemical Hashed Fingerprinting to determine the structural similarity of each compound C1-C20 to those contained in each database. This score was then averaged to see the mean similarity to known RNA binders.

TABLE 4
Summary of Oligonucleotides used

| | | | SEQ | |
|---|---|---|---|---|
| | | | ID | |
| Oligonucleotide | Sequence 5′ -> 3′ | Experiment | NO: | Supplier |
| RNU6 | ACACGCAAATTCGTGAAGCGTTC | RT-qPCR | 1 | IDT |
| miR-155-5p Fwd. | TTAATGCTAATCGTGATAGGGGTT | RT-qPCR | 2 | IDT |
| Universal Reverse | GAATCGAGCACCAGTTACGC | RT-qPCR | 3 | IDT |
| Pri-miR-155 Fwd. | GTGTATGATGCCTGTTACTAGCA | RT-qPCR | 4 | IDT |
| Pri-miR-155 Rev | GCCTGAAGTCTAAGTTTATCCAGC | RT-qPCR | 5 | IDT |
| Pre-miR-155 Fwd. | TGCTAATCGTGATAGGGGTTTT | RT-qPCR | 6 | IDT |
| Pre-miR-155 Rev | TGCTAATATGTAGGAGTCAGTTGG | RT-qPCR | 7 | IDT |
| | A | | | |
| Pre-miR-155 | CTGTTAATGCTAATCGTGATAGGGG | In vitro | 8 | IDT |
| Template | TTTTTGCCTCCAACTGACTCCTACA | cleavage | | |
| | TATTAGCATTAACAG | | | |
| Pre-miR-155 A19 | CTGTTAATGCTAATCGTGATAGGGG | In vitro | 9 | IDT |
| mutant Template | TTTTTGCCTCCAACTGACTCCTATC | cleavage | | |
| | ATATTAGCATTAACAG | | | |
| Pre-miR-155 U29 | CTGTTAATGCTAATCGTGATAGGGG | In vitro | 10 | IDT |
| mutant Template | TTAGTTGCCTCCAACTGACTCCTAC | cleavage | | |
| | ATATTAGCATTAACAG | | | |
| Pre-miR-155 T7 | GGCCGGATCCTAATACGACTCACT | In vitro | 11 | IDT |
| Fwd. | ATAGGCTGTTAATGCTAATCGTGAT | cleavage | | |
| | AGGG | | | |
| Pre-miR-155 A19 | CTGTTAATGCTAATATGATAGGAGT | In vitro | 12 | IDT |
| Mutant T7 Fwd. | CAG | cleavage | | |
| Pre-miR-155 A | GGCCGGATCCTAATACGACTCACT | Binding | 13 | IDT |
| Bulge Fwd. | ATAGGGAGAGGGUUUAAU | | | |
| Pre-miR-155 A | CCTTGCGGATCCAAT | Binding | 14 | IDT |
| Bulge Rev. | | | | |
| Cy5-155-A | Cy5- | MST Binding | 15 | Dharmacon |
| | GUAAUGAUUACGAAAGUAACAUU | | | |
| | GC | | | |
| Cy5-155-AU | Cy5- | MST Binding | 16 | Dharmacon |
| | GUAAUGAUUACGAAAGUAAUCAU | | | |
| | UGC | | | |
| Cy5-155-C | Cy5- | MST Binding | 17 | Dharmacon |
| | GUAAUUCGUACGAAAGUAUAAUU | | | |
| | GC | | | |
| Cy5-155-GC | Cy5- | MST Binding | 18 | Dharmacon |
| | GUAAUUCGUACGAAAGUAUGAAU | | | |
| | UGC | | | |
| 18S Fwd. | GTAACCCGTTGAACCCCATT | RT-qPCR | 19 | IDT |
| 18S Rev | CCATCCAATCGGTAGTAGCG | RT-qPCR | 20 | IDT |
| GAPDH Fwd. | GTTCGACAGTCAGCCGCATC | RT-qPCR | 21 | IDT |
| GAPDH Rev | GGAATTTGCCATGGGTGGA | RT-qPCR | 22 | IDT |
| Control Screen | GGGAGAGGGTTTAATTACGAAAGT | Screening | 23 | IDT |
| Template | ATTGGATCCGCAAGG | | | |
| 3x3 ILL | GGGAGAGGGTTTAATNNNTACGAA | Screening | 24 | IDT |
| | AGTNNNATTGGATCCGCAAGG | | | |
| T7 Forward | GGCCGGATCCTAATACGACTCACT | Screening | 25 | IDT |
| | ATAGGGAGATTTUUUAAU | PCR | | |
| Reverse | CCTTGCGGATCCAAT | Screening | 26 | IDT |
| | | PCR | | |

Bolded nucleotides are mutations, N = any nucleotide A, T, G or C.

LOGO and DiffLOGO RNA Motif Analysis. Z obs scores that were corresponding to the top (enriched) and bottom (discriminated) 0.5 percent were used to select the sequences that were enriched or discriminated. The resulting list of sequences were converted to position weight matrix (PWM) lists for each compound however the “enriched” and “discriminated” sequences were kept separate using JMP®, Version <13.2.1>. SAS Institute Inc., Cary, NC, 1989-2007. The R package Difflogo (Nettling et al, 2015) which a part of Bioconductor, was utilized to create the sequence logos from PWM lists for each compound and visually compare the differences between them. Difflogo was installed on RStudio (version 1.2.5042, RStudio Team 2020) with R 3.6.3 (R Core Team, 2014).¹³

Microscale thermophoresis study of binding. Binding constants were measured by MST using premium capillaries (Nanotemper) using serial dilations of compound from 50 μM to 1 nM in 1:2 dilutions. RNA-Cy5-155-A (20 nM) or Cy5-155-AU (20 nM), Cy5-155-C(20 nM), and Cy5-155-GC (20 nM) were annealed in 2×DNA buffer by heating at 70° C. with cooling to room temperature on the bench. Then the 2× compound and 2× RNA solutions were mixed together in a 1:1 ratio and allowed to equilibrate at room temperature for 10 min. Then, the capillaries were loaded and run on a Nanotemper Monolight 0.115 under previously described conditions.¹⁴ MST power was set to 40% and the LED power was set to 3%.

RT-qPCR analysis for Mature, Primary and Precursor miRNAs and mRNA levels. After compound treatment for the indicated time (MDA-MB-231 48 h and HUVECs 24 h), total RNA was extracted using the Zymo Quick-RNA mini prep kit according to the manufacturers protocol. Reverse Transcription (RT) for microRNAs and pri- and pre-miR-155 were done on 200 ng of RNA using the miScript II RT kit (Qiagen) according to the manufacturer's protocol. The mRNAs were done using QScript (Quanta Bio) on 200 ng of total RNA according to the manufacturer's protocol. RT-qPCR was done as mentioned above. The obtained data was analyzed by using the ΔΔCt method as described previously. For the washout experiment, MDA-MB-231 cells were treated with 3 for 48 h. Then the media was changed with fresh growth medium without compound and the cells incubated for 12, 24 and 36 h with RNA harvested and pre-miR-155 levels analyzed as described above.

Western-Blotting. MDA-MB-231 cells were plated in 6 well plates at 150,000 cells/well. And at 50% confluency, cells were treated with Vehicle or 155-ribotac for 48 h. Then the medium was removed and washed with 1× DPBS. The cells were trypsinized and pelleted. The pellets were washed twice with 1× DPBS and then lysed in 50 L of M-PER (Thermo Fisher) buffer with 1× protease inhibitor cocktail (Roche) on ice for 20 min. The lysate was centrifuged at 4° C. for 15 min and the supernatants were collected. Protein concentration were determined using a Pierce Mico BCA Protein Assay kit (Fisher Scientific) according to the manufacturer's protocol. Approximately 20 g of total protein for each sample was resolved on a 10% SDS-polyacrylamide gel and the protein were then transferred to a PVDF membrane. The membrane was then blocked in 1× TBST (1× TBS with 0.1% of Tween 20) with 5% milk for 40 min at RT. The membrane was then incubated with 1× TBST containing 5% milk with either SOCS1(Cell Signaling Technology, 3950S) primary antibody (1: 1000) at 4° C. overnight. The membrane was then washed 1× TBST for 10 min for 3 times and incubated with 1:2000 anti-rabbit IgG horseradish-peroxidase secondary antibody conjugate (Cell Signaling Technology, 7074S) in 1×TBST with 5% milk for 2 h. After washing with 1× TBST for 15 min for 3 times, SOCS1 protein expression was detected by using SuperSignal West Pico Chemiluminescent Substrate (Pierce Biotechnology).

The membrane was then stripped by Stripping Buffer (200 mM glycine with 0.1% SDS, pH 2.2) for 90 min at RT and then the membrane was washed with 1× TBST for 10 min for 3 times. The membrane was then incubated with 1:10000 j-actin primary antibody (Cell Signaling Technology, 3700S) in 1× TBST containing 5% milk for 2 h at RT, followed by washing with 1× TBST for 10 min for 3 times and incubated with 1:10,000 anti-mouse IgG horseradish-peroxidase secondary antibody conjugate (Cell Signaling Technology, 7076S) in 1× TBST with 5% milk for 1 h. The membrane was then washed with 1× TBST for 10 min for 3 times and j-actin expression level was detected. ImageJ software was used to quantify the protein bands.

Western blot for VHL was performed the same as for SOCS1 with primary antibody(Cell Signaling Technology, 68547S) in a 1:1000 dilution.

Cellular Chem-CLIP and Competitive-Chem-CLIP of pre-miR-155. MDA-MB-231 cells at 80% confluency in 100 mm dish were treated with Chem-CLIP probe at the indicated concentration overnight. Then cells were washed with DPBS and harvested by scrapping. Total RNA was extracted by using the miRNeasy mini kit (Qiagen) according to the manufacture's protocol. Then, 50 g of total RNA was treated to 150 L of Dynabeads MyOne Streptavidin C1 (Invitrogen) slurry in 500 L of 1×DNA buffer (8.0 mM Na₂PO₄, 185 mM NaCl, pH=7.0) and shaked for 6 h. The beads then captured on a Magnetic rack and washed with DPBS for 4 times. The bound RNA was then cleaved from beads in 150 μL of 95% formamide with 10 mM EDTA at 95° C. for 30 min. Released RNA was then cleaned up by Zymo Quick-RNA MiniPrep kit per the manufacture's protocol. RT-qPCR was carried out on pre-miR-155 as previously described above by using 10 ng of input cDNA from before and after pulldown RNA samples. Enrichment of pre-miR-155 was calculated as previously reported¹⁵. Competitive Chem-CLIP was completed by pre-treating MDA-MB-231 cells with compound B for 2 h followed by dosing of Chem-CLIP at 100 nM overnight. Sample preparation and data analysis were performed as for Chem-CLIP.

Migration Assay: The Migration assay was performed the same as previous publication¹⁵. Briefly, MDA-MB-231 cells were serum starved for 12 h in RPMI medium without FBS. Then, samples of 50,000 cells treated with vehicle or 155-Ribotac in serum starved medium were seeded into Hanging cell culture Inserts with 8.0 m pores for 24 well plate with complete growth medium in the bottom well. After 20 h, the medium was aspirated and the inserts and the bottom wells were washed with DPBS twice. Then to the bottom well were added 400 L of 4% paraformaldehyde. After fixing for 20 min. The inserts and wells were washed with DPBS twice and then treated with 400 L of 0.1% crystal violet solution. After 20 min, the wells and inserts were washed with water twice and PBS once. Cotton swabs were used to remove the cells inside the inserts. Migration inserts were completely air-dried and imaged by using a Leica DMI3000 B upright fluorescent microscope.

Migration of MCF-10a cells were performed the same as for MDA-MB-231 cells after transfection of the indicated plasmids by Lipofectamine RNAiMAX per the manufacture's protocol.

Immunoprecipitation of RNase L. RNase L immunoprecipitation is performed according to published procedures.¹⁶ Briefly, at -60% confluency, MDA-MB-231 cells were treated with vehicle, 100 nM of 3 or 5 for 48 h. Cells were then washed with DPBS, and detached by scraper. The collected cells were lysed in 100 L of M-PER buffer(78503, Thermo Fisher) containing 1× Protease Inhibitor Cocktail III for Mammalian Cells (Research Products International Corp.) and 80 U of RNaseOUT Recombinant Ribonuclease Inhibitor (Invitrogen) per the manufacture's protocol. The samples were centrifuged at 13,000×g at 4° C. Protein concentration was determined by using a Pierce Mico BCA Protein Assay kit (Fisher Scientific). Approximately 200 g of the total protein per sample was incubated at 4° C. overnight with Dynabeads Protein A (Life Technologies) bound to either RNase L mouse primary antibody (Santa Cruz Biotechnology: sc-74405) or j-actin mouse primary antibody (Cell signaling: 8H10D10). Beads were then washed with 1× DPBST (1× DPBS supplemented with 0.02% Tween-20) for 3 times, after which RNA was extracted by using a miRNeasy Mini Kit (Qiagen) per the manufacturer's instructions. RT-qPCR was performed the same as mentioned above.

RT-qPCR analysis for Mature, Primary and Precursor miRNAs and mRNA levels part. Relative RNA expression was determined by using ΔΔCt method with 18S rRNA as an internal control. Normalized fold change was calculated with the equation as below:

$$Normalized Fold Change = \frac{Relative RNA Expression in RNase L fraction}{Relative RNA Expression in β - actin fraction}$$

Global proteomics profiling by using LC-MS/MS. MDA-MB-231 cells were resolubilized in 1× DPBS, lysed via sonication and protein samples were obtained by centrifuge. Protein concentration was measured using a Bradford assay (Bio-Rad). The protein samples (20 μg) obtained above were denatured with 6 M urea in 50 mM NH₄HCO₃, reduced with 10 mM tris(2-carboxyethyl)phosphine hydrochloride (TCEP) for 30 min, and then alkylated with 25 mM iodoacetamide for 30 min in dark. Then the samples were diluted to 2 M urea solution with 50 mM NH₄HCO₃, and then digested with trypsin (1 L of 0.5 g/L) in the presence of 1 mM CaCl₂) for 12 h at 37° C. Samples were acidified with acetic acid to a final concentration of 5%, desalted over a self-packed C18 spin column, and dried. Samples were analyzed by LC-MS/MS and the MS data was processed with MaxQuant as described previously¹⁵.

Global Proteomics. Global proteomics data were acquired as previously described.¹⁵ Data obtained from the LC-MS-MS run were analyzed as indicated below:

MaxQuant analysis. The MS data was analyzed with MaxQuant¹⁷ (V1.6.1.0) and searched against the human proteome (Uniprot) and a common list of contaminants (included in MaxQuant). The first peptide search tolerance was set at 20 ppm, 10 ppm was used for the main peptide search and fragment mass tolerance was set to 0.02 Da. The false discovery rate for peptides, proteins and sites identification was set to 1%. The minimum peptide length was set to 6 amino acids and peptide re-quantification and label-free quantification (MaxLFQ) were enabled. The minimal number of peptides per protein was set to two. Methionine oxidation was searched as a variable modification and carbamidomethylation of cysteines was searched as a fixed modification.

TABLE 1
Hit compounds from 2DCS studies.

| | | |
|---|---|---|
| 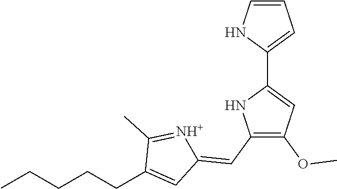 | C1 | Bipyrrol pyrrolium salts |
| 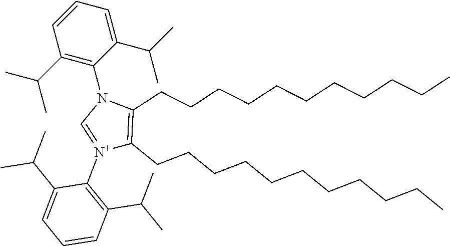 [SMILES: CCCCCCCCCCCc1c(CCCCCCCCCCC)[n+](-c2c(C(C)C)cccc2C(C)C)cn1-c1c(C(C)C)cccc1C(C)C] | C2 (1) | Azolium salts |
| 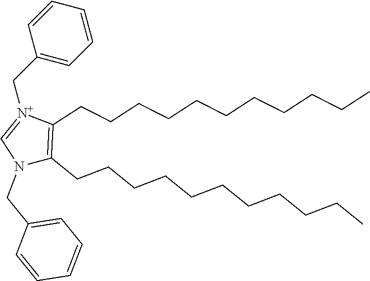 [SMILES: CCCCCCCCCCCc1c(CCCCCCCCCCC)[n+](Cc2ccccc2)cn1Cc1ccccc1] | C3 | Azolium salts |
| 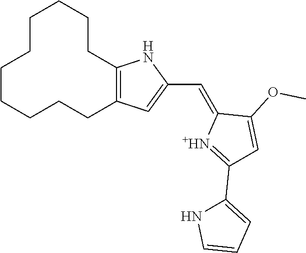 [SMILES: COC1=CC(c2ccc[nH]2)=[NH+]/C1=C\c1cc2c([nH]1)CCCCCCCCCC2] | C4 | Bipyrrolo pyrrolium salts |
| 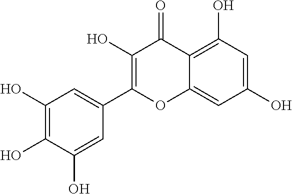 [SMILES: O=c1c(O)c(-c2cc(O)c(O)c(O)c2)oc2cc(O)cc(O)c12] | C5 | Chromone |
| 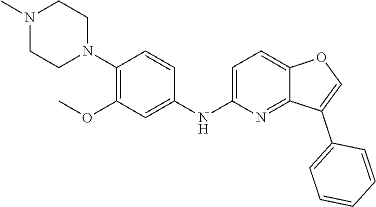 [SMILES: COc1cc(Nc2ccc3occ(-c4ccccc4)c3n2)ccc1N1CCN(C)CC1] | C6 | 3- phenylfuro[3,2- b]pyridine-5- amines |
| 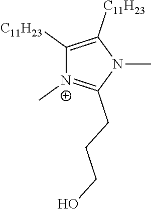 | C7 | Azolium salts |
| 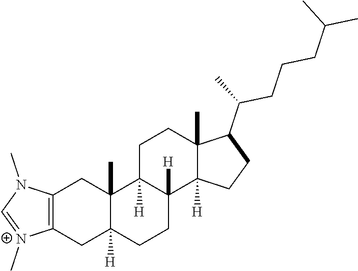 | C8 | Azolium salts |
| 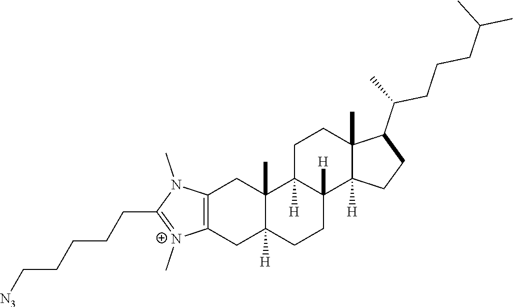 | C9 | Azolium salts |
| 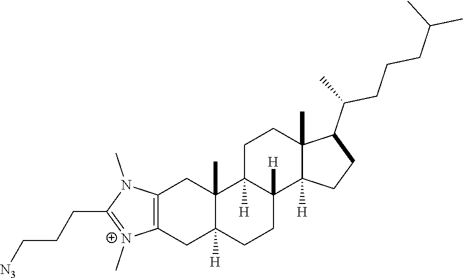 | C10 | Azolium salts |
| 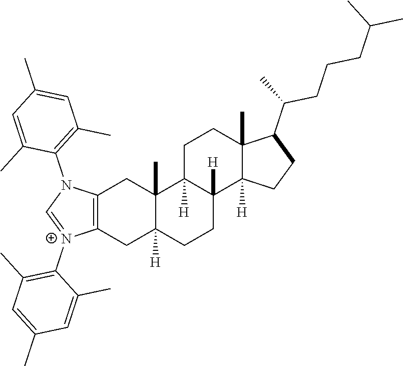 | C11 | Azolium salts |
| 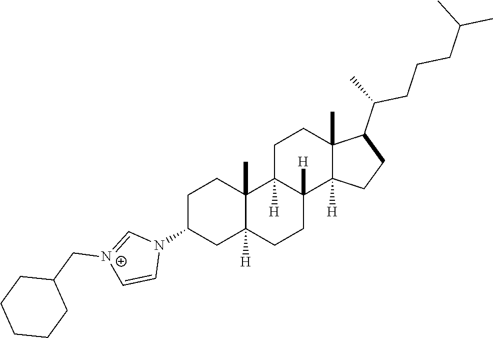 | C12 | Azolium salts |
| 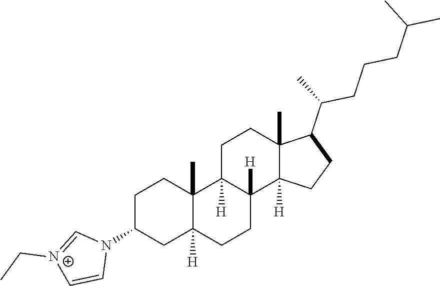 | C13 | Azolium salts |
| 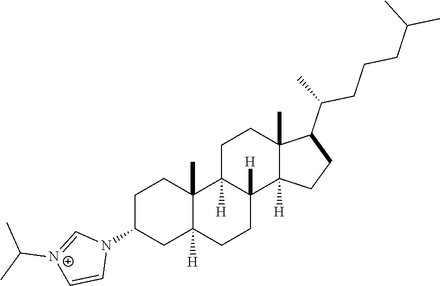 | C14 | Azolium salts |
| 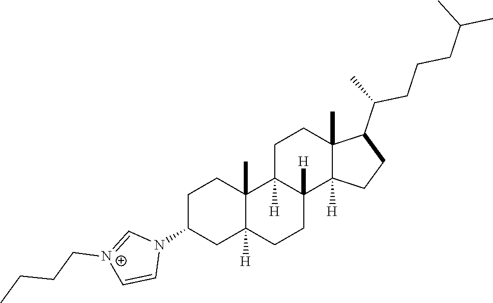 | C15 | Azolium salts |
| 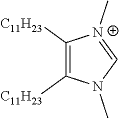 | C16 | Azolium salts |
| 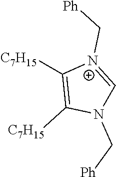 | C17 | Azolium salts |
| 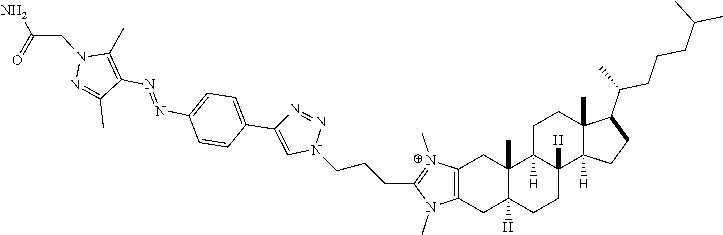 | C18 | Azolium salts |
| 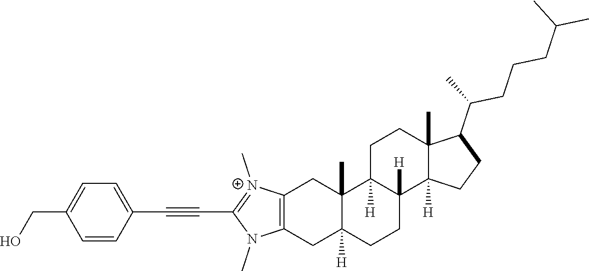 | C19 | Azolium salts |
| 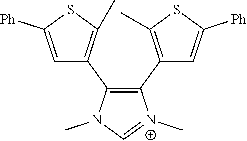 [SMILES: Cc1sc(-c2ccccc2)cc1-c1c(-c2cc(-c3ccccc3)sc2C)[n+](C)cn1C] | C20 | Azolium salts |

TABLE 2
Physicochemical properties of 2DCS hits and known RNA binders
found within Inforna and R-BIND, and protein binders.

| | | | | Protein |
|---|---|---|---|---|
| Property | All 2DCS Hits | Inforna | R-BIND | Binders |
| Molecule | 498.3 | 457.3 | 349 | 439.5 |
| Weight (Da) | | | | |
| cLogP | 5.8 | 0.2 | 1.7 | 3.26 |
| Total Polar | 33.4 | 156.1 | 74.6 | 92 |
| Surface Area | | | | |
| (Å²) | | | | |
| Number of H- | 0.8 | 5.2 | 2.7 | 1.8 |
| Bond Donor | | | | |
| Number of H- | 1.4 | 8.6 | 5.9 | 5.2 |
| Bond Acceptor | | | | |
| Number of | 10.4 | 7.4 | 5.5 | 6.0 |
| Rotatable Bonds | | | | |
| Number of | 4.2 | 4.2 | 0.24 | 0.96 |
| Chiral Center | | | | |
| Number of | 11.5 | 19.7 | 15 | 16.0 |
| Aromatic Atoms | | | | |
| Number of | 24.9 | 13 | 10.6 | 15.1 |
| Aliphatic Atoms | | | | |
| Number of | 4.4 | 3.9 | 3.7 | 4 |
| Rings | | | | |
| Number of | 62.2 | 32.7 | 25.6 | 40 |
| Heavy Atom | | | | |

Inforna (n = 402); R-BIND (n = 104); Protein Binders from Binding DB (n = 272,583).
Bold/Italic = comparison to Inforna, Underline = comparison to protein binders.

TABLE 3
Motif analysis of Inforna and hits obtained from screening.

| | | | Motifs in 3 × 3 | |
|---|---|---|---|---|
| | Motifs in | Motifs in | study by Haniff | Motifs in |
| Motif Type | Inforna | 2DCS Hits | et al.¹ | 3 × 3 ILL |
| 3′ 1 nt. Bulge | 2,647 | 275 | 769 | 220 |
| 3′ 2 nt. Bulge | 731 | 0 | 0 | 0 |
| 3′ 3 nt. Bulge | 89 | 0 | 0 | 0 |
| 3′ 4 nt. Bulge | 79 | 0 | 0 | 0 |
| 3′ 9 nt. Bulge | 3 | 0 | 0 | 0 |
| 5″ 1 nt. Bulge | 50,264 | 270 | 762 | 211 |
| 5′ 2 nt. Bulge | 1,009 | 0 | 0 | 0 |
| 5′ 3 nt. Bulge | 47 | 0 | 0 | 0 |
| 5′ 4 nt. Bulge | 31 | 0 | 0 | 0 |
| 5′ 5 nt. Bulge | 18 | 0 | 0 | 0 |
| 5′ 7 nt. Bulge | 14 | 0 | 0 | 0 |
| 5′ 8 nt. Bulge | 6 | 0 | 0 | 0 |
| HP 3 nt. | 665 | 0 | 0 | 0 |
| HP 4 nt. | 529 | 0 | 0 | 0 |
| HP 5 nt. | 3,126 | 0 | 0 | 0 |
| HP 6 nt. | 1,821 | 0 | 0 | 0 |
| HP 7 nt. | 6 | 0 | 0 | 0 |
| HP 8 nt. | 6 | 0 | 0 | 0 |
| HP 10 nt. | 2 | 0 | 0 | 0 |
| HP 12 nt. | 1 | 0 | 0 | 0 |
| HP 13 nt. | 1 | 0 | 0 | 0 |
| HP 26 nt. | 1 | 0 | 0 | 0 |
| 1 × 1 IL | 13,844 | 549 | 6,819 | 1036 |
| 1 × 2 IL | 31 | 0 | 0 | 0 |
| 1 × 3 IL | 42 | 0 | 0 | 0 |
| 2 × 1 IL | 15,827 | 20 | 0 | 0 |
| 2 × 2 IL | 2,439 | 1,501 | 1,634 | 1192 |
| 2 × 6 IL | 15 | 0 | 0 | 0 |
| 3 × 1 IL | 11 | 0 | 0 | 0 |
| 3 × 2 IL | 9,434 | 0 | 0 | 0 |
| 3 × 3 IL | 2,948 | 3,352 | 1,755 | 1437 |
| 4 × 3 IL | 6,324 | 0 | 0 | 0 |
| 4 × 4 IL | 28 | 0 | 0 | 0 |
| 3 × 1 IL | 2 | 0 | 0 | 0 |
| 4 × 6 IL | 2 | 0 | 0 | 0 |
| 1 × 9 IL | 1 | 0 | 0 | 0 |
| 2 × 3 IL | 36 | 0 | 0 | 0 |
| 3 × 4 IL | 96 | 0 | 0 | 0 |
| 2 × 7 IL | 2 | 0 | 0 | 0 |
| 3 × 6 IL | 1 | 0 | 0 | 0 |
| 6 × 5 IL | 1 | 0 | 0 | 0 |
| 11 × 1 IL | 3 | 0 | 0 | 0 |
| 12 × 2 IL | 4 | 0 | 0 | 0 |
| 6 × 1 IL | 12 | 0 | 0 | 0 |
| Total Motifs | 112,213 | 5,967 | 11,770 | 4,096 |

IL = Internal Loop, HP = Hairpin, and nt. = nucleotide;
****, p < 0.00001 for enrichment between hits in this 3 × 3 screen to those reported by Haniff et al.¹

### EXPERIMENTAL SECTION

### Synthetic Schemes and Methods for Compounds

General Methods. HATU was purchased from Oakwood Products, Inc. Dimethylformamide were purchased from Fisher Chemical. Acetic acid was purchased from Avantor Performance Materials. Propylamine and chlorambucil acid were purchased from Alfa Aesar. N,N-Diisopropylethylamine and n-Butyl Lithium were purchased from Sigma-Aldrich. N-Boc-Ethylenediamine, 5-Carboxyfluorescein, Boc-Lys(Ac)-OH, N-(2-Aminoethyl)biotinamide, and Boc-Lys-OH were purchased from Combi-Blocks. Ethyl bromoacetate, 2,3-Butanedione, and tetrahydrofuran were purchased from Fisher Scientific and 2,6-Diisopropylaniline was purchased from VWR. All chemicals were used as received without further purification.

Reactions were monitored by LC-MS or by thin layer chromatography (TLC, Agela Technologies) with bands visualized under UV light (254 nm). Products were purified by HPLC (waters 2487 and 1525) equipped with a SunFire® Prep C18 OBD™ 5 μm column (19×150 mm). The flow rate used for HPLC purification was 5 mL/min. The purity of the products was analyzed by analytical HPLC (Waters 2487 and 1525) equipped with a SunFire® C18 3.5 μm column (4.6×150 mm). The flow rate for the analytical HPLC is 1 mL/min. The gradients for both purification and purity analysis are from 100% of H₂O with 0.1% TFA to 100% MeOH with 0.1% TFA in 60 min.

NMR spectra were measured by using a 400 UltraShield™ (Bruker) (400 MHz for ¹H and 100 MHz for ¹³C) or an Ascend™ 600(Bruker) (600 MHz for ¹H and 150 MHz for ¹³C). Chemical shifts are reported in ppm with the residual solvents as the internal standards and coupling constants (J values) are reported in hertz. High resolution mass spectrometry was obtained by using an Agilent 1260 Infinity LC system coupled to an Agilent 6230 TOF(HR-ESI). The LC system was equipped with a Poroshell 120 EC-C18 column (Agilent, 50 mm×4.6 mm, 2.7 m). MALDI was performed on a 4800 Plus MALDI TOF/TOF Analyzer

### Synthetic Schemes and Methods

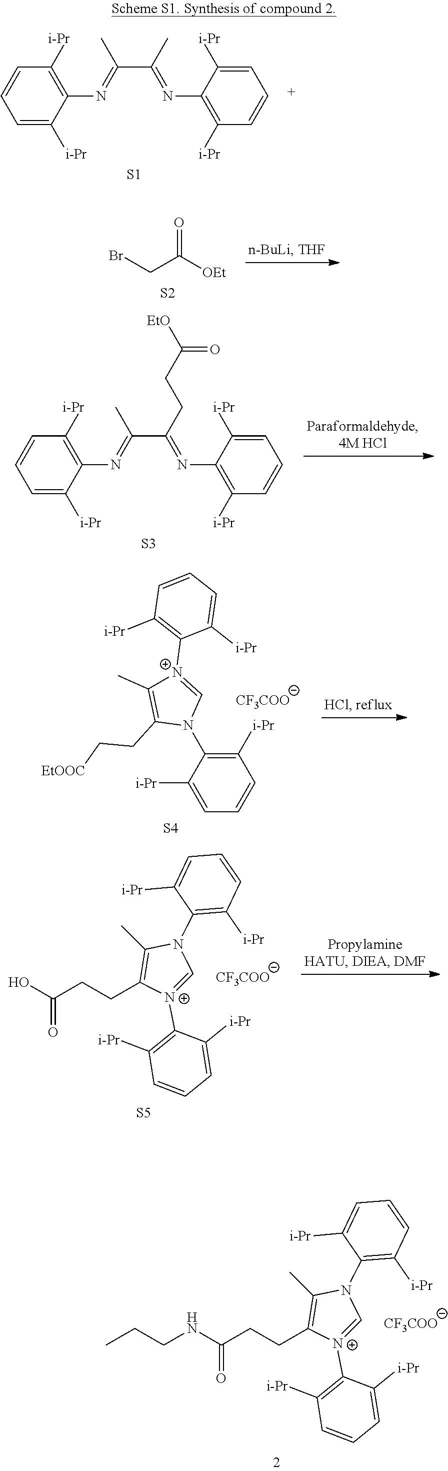

Compound S4. Compound S3 was synthesized as literature¹⁸. To a solution of S3 (404 mg, 1 mmol) in dry THF at −80° C. was added 500 L of n-BuLi (2 M, 500 μL, 1 mmol) and the mixture was allowed to stirred at −80° C. for 20 min and then at RT for 20 min. Then the mixture was cooled to −80° C. again, followed by the addition of ethyl bromoacetate (167 mg, 1 mmol). The mixture was allowed to warm to RT and stirred for another 4 h. The reaction mixture was then concentrated in vacuo and the residue was re-suspended in 4 M HCl in Dioxane, followed by addition of paraformaldehyde (58 mg, 2 mmol). The reaction mixture was stirred overnight at RT and concentrated in vacuo. The residue was purified by HPLC to give the ester as a white solid (100 mg, 0.162 mmol, 16%)¹H NMR (400 MHz, CDCl₃) δ (ppm) 9.38 (s, 1H), 7.60 (m, 2H) 7.38 (m, 4H) 4.09 (q, J=7.12 Hz, 2H), 2.83 (t, J=7.04 Hz, 2H), 2.38 (t, J=7.04 Hz, 2H), 2.27 (m, 4H), 2.17 (s, 3H), 1.33 (d, J=6.8 Hz, 6H), 1.25 (d, J=6.8 Hz, 6H), 1.22 (d, J=7.12 Hz, 3H), 1.17 (m, 12H); ¹³C NMR (100 MHz, CDCl₃) δ (ppm) 171.0, 145.2, 145.1, 136.7, 132.6, 132.4, 130.9, 130.9, 127.5, 125.2, 125.0, 61.1, 29.2, 29.1, 25.8, 24.7, 23.2, 22.5, 18.8, 14.1, 9.2; HRMS (m/z): calculated for C₃₃H₄₇N₂O₂ [M]⁺ 503.3632, found:.503.3597.

Compound S5. A solution of S4 (100 mg, 0.162 mmol) in 3 mL of conc. HCl was stirred at reflux overnight. The mixture was concentrated in vacuo to give compound S5 as a white solid without further purification.

Compound 2. A solution of S5 (3 mg, 0.005 mmol), propylamine (0.3 mg, 0.015 mmol), HATU (3.8 mg, 0.01 mmol) and DIEA (1.9 mg, 0.015 mmol) in DMF was stirred at RT for 2h. Compound 2 was purified by HPLC as a white solid(2.1 mg, 0.0033 mmol, 66%). ¹H NMR (400 MHz, CDCl₃) δ (ppm) 8.04 (s, 1H), 7.65 (br, 1H), 7.63 (m, 2H), 7.40 (m, 4H), 3.11 (m, 2H), 2.91 (t, J=7.8 Hz, 2H), 2.50 (t, J=7.8 Hz, 2H), 2.43 (m, 2H) 2.34 (m, 2H), 2.21 (s, 3H), 1.49 (m, 2H), 1.35 (d, J=6.7 Hz, 6H), 1.30 (d, J=6.8 Hz, 6H), 1.16 (d, J=6.9 Hz, 6H), 1.13 (d, J=6.9 Hz, 6H), 0.87 (t, J=7.4 Hz, 3H); ¹³C NMR (150 MHz, CDCl₃) δ (ppm) 170.8, 145.8, 145.7, 133.6, 133.0, 132.9, 132.7, 131.7, 127.5, 125.5, 125.3, 41.6, 33.3, 29.1, 29.1, 26.1, 25.3, 23.3, 22.8, 22.5, 19.9, 11.5, 9.2; HRMS (m/z): calculated for C₃₄H₅₀N₃O [M]⁺516.3948, found:516.3897.

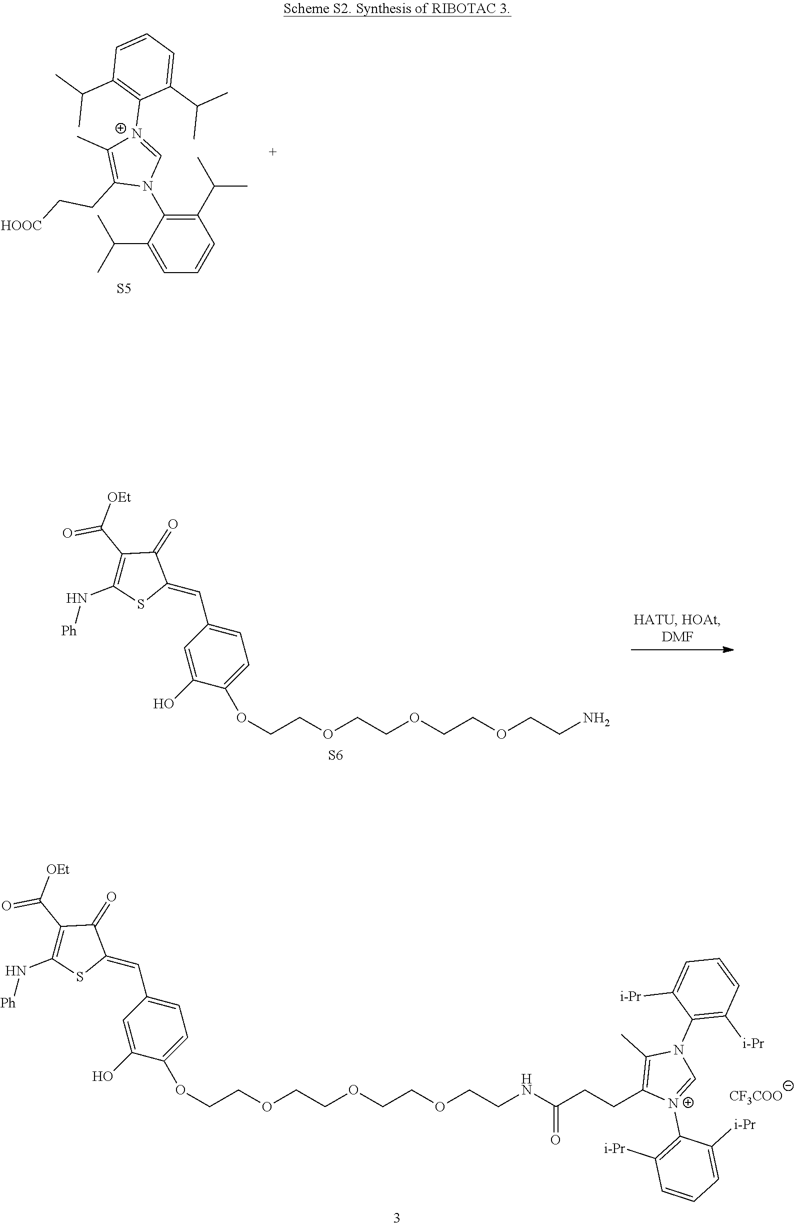

Compound 3. A solution of S5 (3 mg, 0.005 mmol), HATU(3.8 mg, 0.01 mmol) and DIEA(1.9 mg, 0.015 mmol) in 0.2 mL of DMF was stirred at RT for 20 min and then S6 (5.7 mg, 0.01 mmol) was added and the mixture was stirred at RT for another 2 h. The product was purified by HPLC to afford 3 as a TFA salt (1.3 mg, 0.0012 mmol, 23%). ¹H NMR (600 MHz, CD₃OD) δ (ppm) 9.7(s, 1H), 7.64-7.68 (m, 3H), 7.45-7.57 (m, 9H), 6.98-7.02 (m, 2H), 4.39(q, J=7.1 Hz, 2H), 4.16-4.22 (m, 2H), 3.84-3.88 (m, 2H), 3.68-3.72 (m, 2H), 3.63-3.66 (m, 2H), 3.59-3.63 (m, 2H), 3.54-3.58 (m, 2H), 3.48 (t, J=5.4 Hz, 2H), 3.28 (t, J=5.4 Hz, 2H), 2.81 (t, J=7.4 Hz, 2H), 2.41-2.47 (m, 2H), 2.35-2.41 (m, 2H), 2.32 (t, J=7.5 Hz, 2H), 2.13(s, 3H), 1.39 (t, J=7.1 Hz, 3H), 1.34 (d, J=6.7 Hz, 6H), 1.29 (d, J=6.8 Hz, 2H), 1.20 (d, J=6.9 Hz, 6H), 1.17 (d, J=6.9 Hz, 6H); ¹³C NMR (100 MHz, CD₃OD) δ (ppm) 184.5, 178.1, 172.5, 167.1, 150.2, 148.5, 147.1, 147.0, 138.8, 138.5, 133.7, 133.5, 133.3, 133.0, 132.1, 131.0, 129.5, 129.3, 129.3, 128.5, 126.4, 126.3, 126.2, 124.8, 117.5, 114.5, 99.0, 71.6, 71.6, 71.5, 71.2, 70.6, 70.4, 69.4, 61.4, 40.5, 33.8, 30.2, 20.2, 26.2, 25.4, 23.4, 22.9, 20.4, 14.8, 9.4.

HRMS (m/z): calculated for C₅₉H₇₅N₄O₉S [M]⁺ 1015.5249, found: 1015.5204.

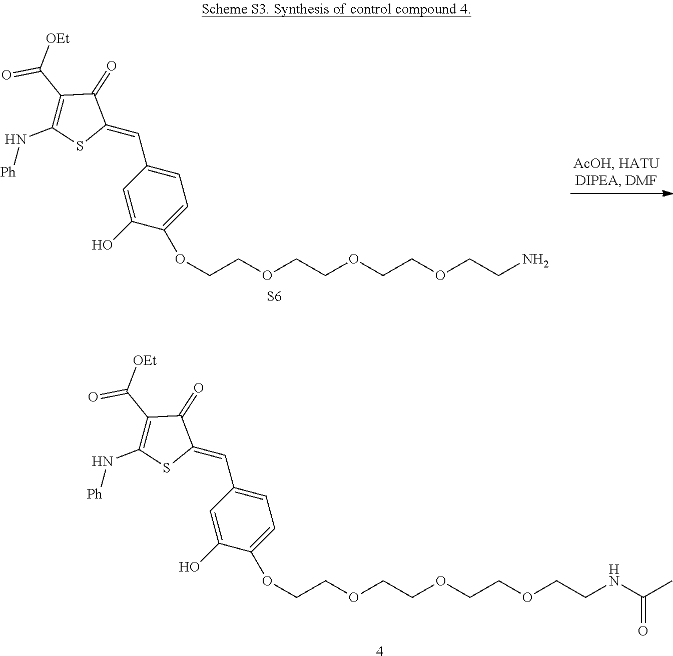

Compound 6. To a solution of DIPEA (5 uL, 0.03 mmol) and HATU (5.7 mg, 0.015 mmol) in 0.5 mL of DMF was added 10 μL of acetic acid solution (1 M of AcOH in DMF). After stirring at RT for 10 min, S6 (5.6 mg, 0.01 mmol) was added to the mixture. The reaction was stirred at RT overnight and then purified by HPLC to give 4 as a TFA salt (3.4 mg, 0.0056 mmol, 56%). ¹H NMR (600 MHz, CD₃OD) 7.66 (s, 1H), 7.53-7.58 (m, 2H), 7.44-7.53 (m, 3H), 6.99-7.07 (m, 3H), 4.38(q, J=7.1 Hz, 2H), 4.18-4.23 (m, 2H), 3.82-3.90 (m, 2H), 3.69-3.74 (m, 2H), 3.65-3.68 (m, 2H), 3.56-3.60 (m, 2H), 3.50 (t, J=5.6 Hz, 2H), 1.91 (s, 3H), 1.39 (t, J=7.1 Hz, 3H). ¹³C NMR (150 MHz, CD₃OD) δ (ppm) 183.1, 176.9, 172.0, 148.8, 147.1, 137.4, 131.7, 129.6, 128.1, 127.1, 125.0, 124.9, 123.4, 116.1, 113.0, 97.5, 70.2, 70.1, 69.8, 69.2, 69.1, 67.9, 60.0, 39.1, 21.1, 13.4. HRMS (m/z): calculated for C₃₀H₃₇N₂O₉S [M+H]⁺ 601.2214, found: 601.2122.

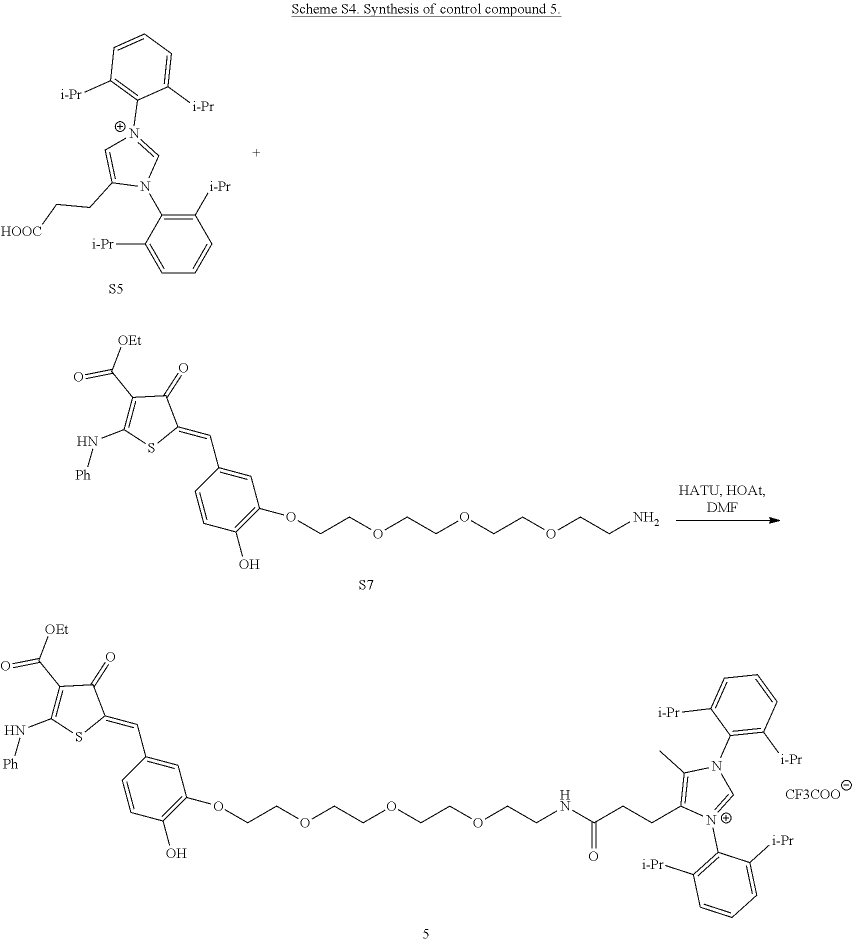

Compound 5. S5 (3 mg, 0.005 mmol), HATU (3.8 mg, 0.01 mmol) and DIEA (1.9 mg, 0.015 mmol) in 0.2 mL of DMF was stirred at RT for 20 min and then S6 (5.7 mg, 0.01 mmol) was added and the mixture was stirred at RT for another 2 h. The product was purified by HPLC to afford the 5 as a TFA salt (1.5 mg, 0.0014 mmol, 27%). ¹H NMR (600 MHz, CD₃OD) δ (ppm) 9.75 (s, 1H), 7.64-7.70 (m, 3H), 7.43-7.58 (m, 9H), 7.15 (d, J=2.0 Hz, 1H), 7.07(dd, J=8.3 Hz, 2 Hz, 1H), 6.83 (d, J=8.3 Hz, 1H), 4.38(q, J=7.1 Hz, 2H), 4.13-4.18 (m, 2H), 3.81-3.86 (m, 2H), 3.66-3.70 (m, 2H), 3.62-3.65 (m, 2H), 3.58-3.62 (m, 2H), 3.54-3.58 (m, 2H), 3.48 (t, J=5.4 Hz, 2H), 3.28 (t, J=5.2 Hz, 2H), 2.81 (t, J=7.4 Hz, 2H), 2.41-2.50 (m, 2H), 2.34-2.41 (m, 2H), 2.32 (t, J=7.4 Hz, 2H), 2.13 (s, 3H), 1.39 (t, J=7.1 Hz, 3H), 1.34 (d, J=6.8 Hz, 6H), 1.29 (d, J=6.8 Hz, 2H), 1.19 (d, J=6.8 Hz, 6H), 1.17 (d, J=6.8 Hz, 6H); ¹³C NMR (150 MHz, CD₃OD) δ (ppm) 184.5, 178.0, 172.5, 167.1, 151.0, 148.4, 147.1, 147.0, 138.8, 138.5 133.6, 133.5, 133.3, 133.3, 132.1, 131.0, 129.5, 129.3, 129.3, 126.9, 126.4, 126.3, 126.2, 125.7, 125.7, 117.4, 99.0, 71.6, 71.6, 71.5, 70.7, 70.4, 69.7, 61.4, 40.5, 33.8, 30.2, 30.2, 26.2, 25.4, 23.4, 22.9, 20.4, 14.8, 9.4. HRMS (m/z): calculated for C₅₉H₇₅N₄O₉S [M]⁺ 1015.5249, found: 1015.5161.

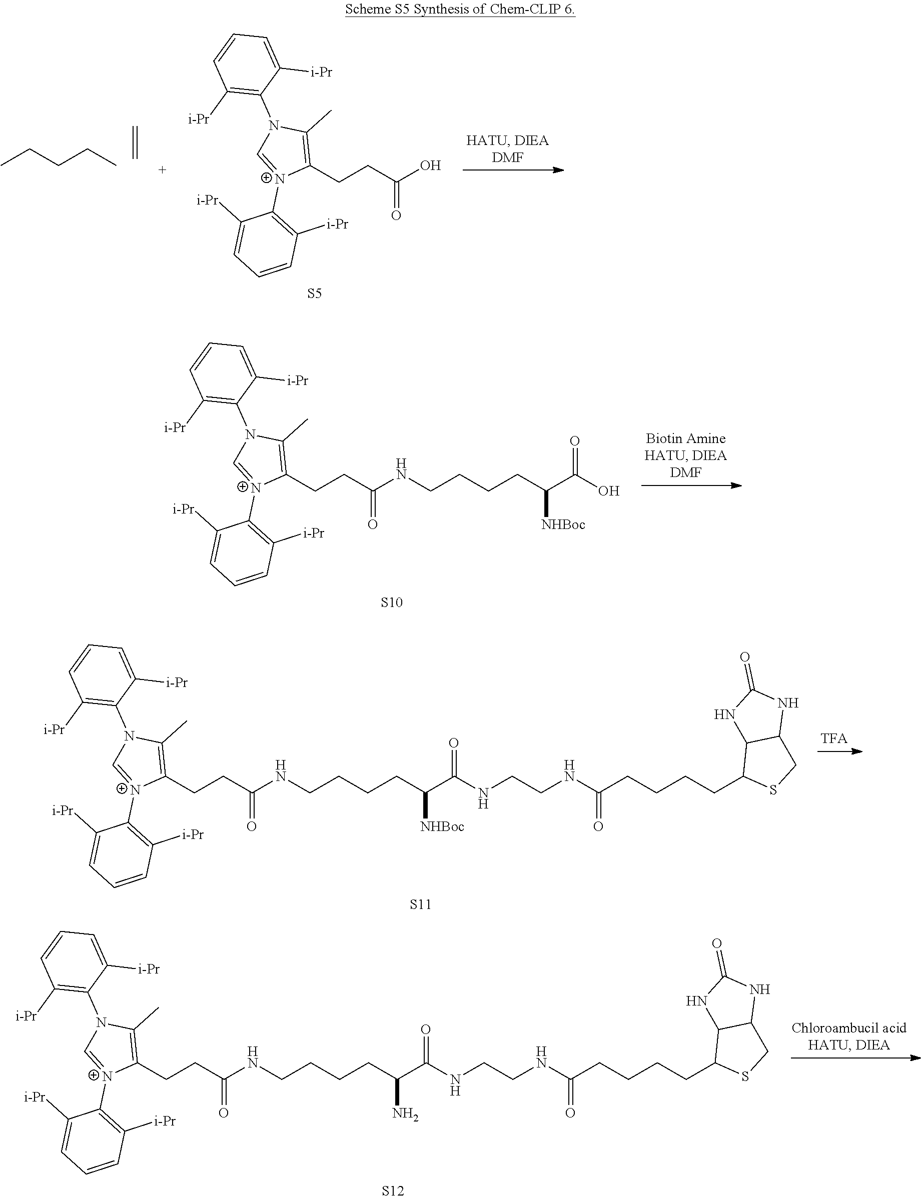

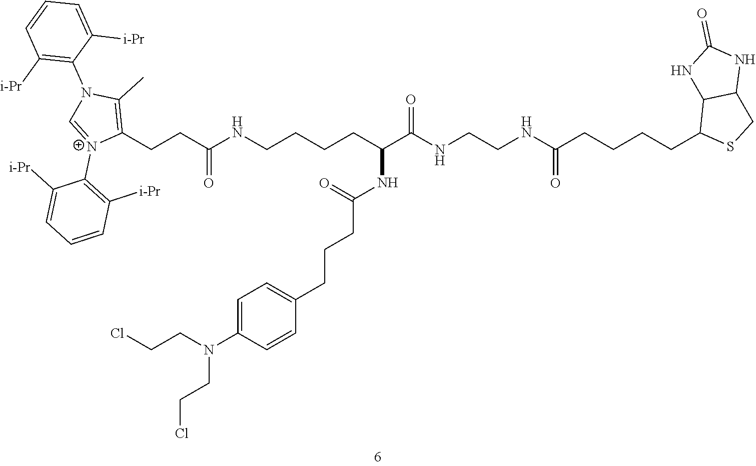 [SMILES: Cc1c(CCC(=O)NCCCC[C@H](NC(=O)CCCc2ccc(N(CCCl)CCCl)cc2)C(=O)NCCNC(=O)CCCCC2SCC3NC(=O)NC32)[n+](-c2c(C(C)C)cccc2C(C)C)cn1-c1c(C(C)C)cccc1C(C)C]

Compound 6. A solution of S5 (3 mg, 0.005 mmol), HATU (2.3 mg, 0.006 mmol) and DIEA (2 mg, 0.015 mmol) in 0.2 mL of DMF was stirred at r.t. for 30 min and then the Boc-Lys-OH was added and the mixture was stirred for another 30 min. The product was purified by HPLC to give 510. A solution of 510, biotin-amine (2.9 mg, 0.01 mmol), HATU (3.8 mg, 0.01 mmol) and DIEA (2 mg, 0.015 mmol) in 0.2 mL of DMF was stirred at r.t. for 2 h which was then subjected for HPLC purification to give 512. A solution of S12 in 30% TFA in DCM was stirred at RT for 30 min and concentrated in vacuo. The residue was directed used in the next step without further purification. A solution of chlorambucil acid (3 mg, 0.01 mmol), HATU (3.8 mg, 0.01 mmol) and DIEA (2 mg, 0.015 mmol) in 0.2 mL DMF was stirred at RT for 20 min and then the amine obtained above in 0.1 mL of DMF was added. The mixture was stirred at RT for another 30 min. The Chem-CLIP probe 6 was obtained by HPLC purification (0.6 mg, 0.45 mol).

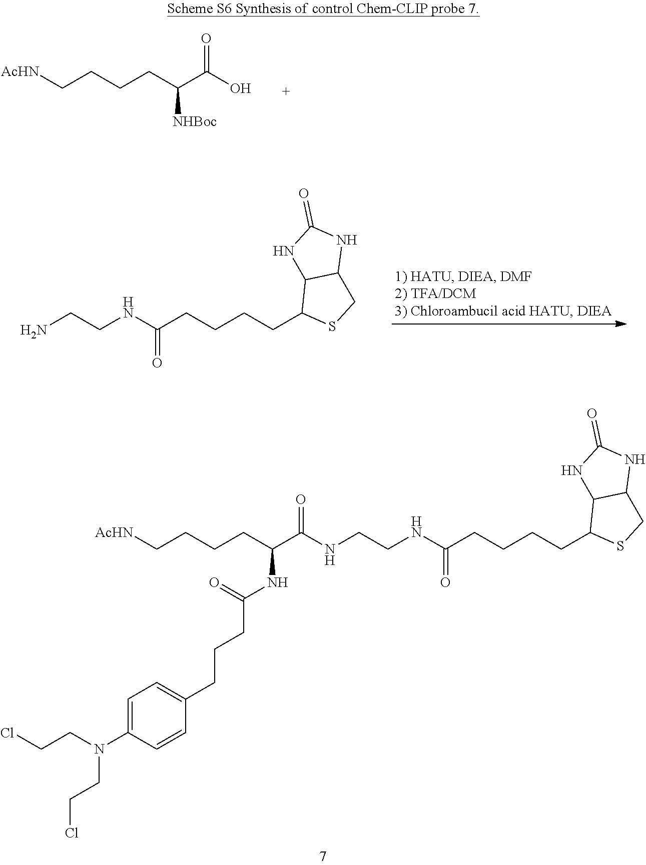 [SMILES: CC(=O)NCCCC[C@H](NC(=O)OC(C)(C)C)C(=O)O ; NCCNC(=O)CCCCC1SCC2NC(=O)NC21 ; CC(=O)NCCCC[C@H](NC(=O)CCCc1ccc(N(CCCl)CCCl)cc1)C(=O)NCCNC(=O)CCCCC1SCC2NC(=O)NC21]

Compound 7: A solution of Boc-Lys(Ac)-OH (5.7 mg, 0.02 mmol), Biotin amine(5.7 mg, 0.02 mmol), HATU (8.4 mg, 0.022 mmol) and DIPEA (3.97 mg, 0.03 mmol) in 0.11 mL of DMF was stirred at RT for 2 h and then the mixture was concentrated in vacuo and the residue was re-suspended in 1 mL of 30% TFA in DCM. The mixture was stirred at RT for another 30 min and then dried in vacuo. A solution of Chloroambucil acid (12.2 mg, 0.04 mmol), HATU(22.8 mg, 0.06 mmol) and DIPEA(9.9 L, 0.06 mmol) in 0.2 mL of DMF was stirred at RT for 20 min, followed by the addition of the crude primary amine. The mixture was stirred at RT for another 30 min and then purified by HPLC to give the control Chem-CLIP probe (0.3 mg, 0.34 mol).

### REFERENCES FOR METHODS AND EXPERIMENTAL SECTIONS

1. Hafeez, H.; Laurent, K.; Xiaohui, L.; Gogce, C.; Jonas, B.; Daniel, A.; Alexander, A.; Malin, L.; Matthew, D., Design of a Small Molecule That Stimulates VEGFA Informed from an Expanded Encyclopedia of RNA Fold-Small Molecule Interactions. Nat. Chem., 2020; p In Press.
2. Huang, Z.; Shi, J.; Gao, Y.; Cui, C.; Zhang, S.; Li, J.; Zhou, Y.; Cui, Q., HMDD v3.0: a database for experimentally supported human microRNA-disease associations. Nucleic Acids Res. 2018, 47 (D1), D1013-D1017.
3. Pathak, S.; Grillo, A. R.; Scarpa, M.; Brun, P.; D′Inca, R.; Nai, L.; Banerjee, A.; Cavallo, D.; Barzon, L.; Palu, G.; Sturniolo, G. C.; Buda, A.; Castagliuolo, I., MiR-155 modulates the inflammatory phenotype of intestinal myofibroblasts by targeting SOCS1 in ulcerative colitis. Exp. Mol. Med. 2015, 47 (5), e164.
4. Dacic, S.; Kelly, L.; Shuai, Y.; Nikiforova, M. N., miRNA expression profiling of lung adenocarcinomas: correlation with mutational status. Mod. Pathol. 2010, 23 (12), 1577-82.
5. Bofill-De Ros, X.; Santos, M.; Vila-Casadesus, M.; Villanueva, E.; Andreu, N.; Dierssen, M.; Fillat, C., Genome-wide miR-155 and miR-802 target gene identification in the hippocampus of Ts65Dn Down syndrome mouse model by miRNA sponges. BMC Genomics 2015, 16 (1), 907.
6. Guedes, J. R.; Custodia, C. M.; Silva, R. J.; de Almeida, L. P.; Pedroso de Lima, M. C.; Cardoso, A. L., Early miR-155 upregulation contributes to neuroinflammation in Alzheimer's disease triple transgenic mouse model. Hum. Mol. Genet. 2014, 23 (23), 6286-301.
7. Wang, Y.; Fu, J.; Jiang, M.; Zhang, X.; Cheng, L.; Xu, X.; Fan, Z.; Zhang, J.; Ye, Q.; Song, H., MiR-410 is overexpressed in liver and colorectal tumors and enhances tumor cell growth by silencing FHL1 via a direct/indirect mechanism. PLoS One 2014, 9 (10), e108708.
8. Velagapudi, S. P.; Gallo, S. M.; Disney, M. D., Sequence-based design of bioactive small molecules that target precursor microRNAs. Nat. Chem. Biol. 2014, 10 (4), 291-297.
9. Velagapudi, S. P.; Luo, Y.; Tran, T.; Haniff, H. S.; Nakai, Y.; Fallahi, M.; Martinez, G. J.; Childs-Disney, J. L.; Disney, M. D., Defining RNA-Small Molecule Affinity Landscapes Enables Design of a Small Molecule Inhibitor of an Oncogenic Noncoding RNA. ACS Cent. Sci. 2017, 3 (3), 205-216.
10. Velagapudi, S. P.; Costales, M. G.; Vummidi, B. R.; Nakai, Y.; Angelbello, A. J.; Tran, T.; Haniff, H. S.; Matsumoto, Y.; Wang, Z. F.; Chatterjee, A. K.; Childs-Disney, J. L.; Disney, M. D., Approved Anti-cancer Drugs Target Oncogenic Non-coding RNAs. Cell Chem. Biol. 2018, 25 (9), 1086-1094 e7.
11. Velagapudi, S. P.; Pushechnikov, A.; Labuda, L. P.; French, J. M.; Disney, M. D., Probing a 2-aminobenzimidazole library for binding to RNA internal loops via two-dimensional combinatorial screening. ACS Chem. Biol. 2012, 7 (11), 1902-9.
12. Morgan, B. S.; Forte, J. E.; Culver, R. N.; Zhang, Y.; Hargrove, A. E., Discovery of Key Physicochemical, Structural, and Spatial Properties of RNA-Targeted Bioactive Ligands. Angew. Chem. Int. Ed. Engl. 2017, 56 (43), 13498-13502.
13. Nettling, M.; Treutler, H.; Grau, J.; Keilwagen, J.; Posch, S.; Grosse, I., DiffLogo: a comparative visualization of sequence motifs. BMC bioinformatics 2015, 16 (1), 387.
14. Haniff, H. S.; Graves, A.; Disney, M. D., Selective small molecule recognition of RNA base pairs. ACS Combi. Sci. 2018, 20 (8), 482.
15. Costales, M. G.; Hoch, D. G.; Abegg, D.; Childs-Disney, J. L.; Velagapudi, S. P.; Adibekian, A.; Disney, M. D., A designed small molecule inhibitor of a non-coding RNA sensitizes HER2 negative cancers to herceptin. J. Am. Chem. Soc. 2019, 141 (7), 2960-2974.
16. Costales, M. G.; Matsumoto, Y.; Velagapudi, S. P.; Disney, M. D., Small molecule targeted recruitment of a nuclease to RNA. J. Am. Chem. Soc. 2018, 140 (22), 6741-6744.
17. Cox, J.; Mann, M., MaxQuant enables high peptide identification rates, individualized p.p.b.-range mass accuracies and proteome-wide protein quantification. Nat. Biotechnol. 2008, 26 (12), 1367-1372.
18. Keyes, A.; Basbug Alhan, H. E.; Ha, U.; Liu, Y.-S.; Smith, S. K.; Teets, T. S.; Beezer, D. B.; Harth, E., Light as a catalytic switch for block copolymer architectures: metal-organic insertion/light initiated radical (MILRad) polymerization. Macromolecules 2018, 51 (18), 7224-7232.

### SUMMARY STATEMENTS

The inventions, examples, biological assays and results described and claimed herein have may attributes and embodiments include, but not limited to, those set forth or described or referenced in this application.

All patents, publications, scientific articles, web sites and other documents and material referenced or mentioned herein are indicative of the levels of skill of those skilled in the art to which the invention pertains, and each such referenced document and material is hereby incorporated by reference to the same extent as if it had been incorporated verbatim and set forth in its entirety herein. The right is reserved to physically incorporate into this specification any and all materials and information from any such paten, publication, scientific article, web site, electronically available information, text-book or other referenced material or document.

The written description of this patent application includes all claims. All claims including all original claims are hereby incorporated by reference in their entirety into the written description portion of the specification and the right is reserved to physically incorporated into the written description or any other portion of the application any and all such claims. Thus, for example, under no circumstances may the patent be interpreted as allegedly not providing a written description for a claim on the assertion that the precise wording of the claim is not set forth in haec verba in written description portion of the patent.

While the invention has been described in conjunction with the detailed description thereof, the foregoing description is intended to illustrate and not limit the scope of the invention, which is defined by the scope of the appended claims. Thus, from the foregoing, it will be appreciated that, although specific nonlimiting embodiments of the invention have been described herein for the purpose of illustration, various modifications may be made without deviating from the spirit and scope of the invention. Other aspects, advantages, and modifications are within the scope of the following claims and the present invention is not limited except as by the appended claims.

The specific methods and compositions described herein are representative of preferred nonlimiting embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in nonlimiting embodiments or examples of the present invention, the terms “comprising”, “including”, “containing”, etc. are to be read expansively and without limitation. The methods and processes illustratively described herein suitably may be practiced in differing orders of steps, and that they are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by various nonlimiting embodiments and/or preferred nonlimiting embodiments and optional features, any and all modifications and variations of the concepts herein disclosed that may be resorted to by those skilled in the art are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

1. A method for conversion of a biologically inactive miRNA binding moiety A into a biologically active miRNA cleaving compound comprising combining moiety A with an RNase recruiting moiety B through a polyoxyethylene amine linker to form a compound of Formula V 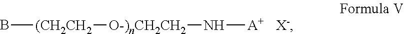
wherein n is an integer of 3 to 5, preferably 3; X⁻ is an organic or inorganic gegenion; and moieties A and B are:

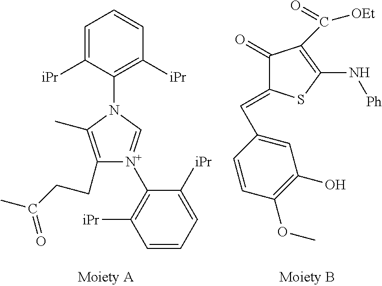 [SMILES: CC(=O)CCc1c(C)n(-c2c(C(C)C)cccc2C(C)C)c[n+]1-c1c(C(C)C)cccc1C(C)C ; CCOC(=O)C1=C(Nc2ccccc2)S/C(=C\c2ccc(OC)c(O)c2)C1=O]

2. A method according to claim 1 wherein Formula V with n as 3 is Formula I

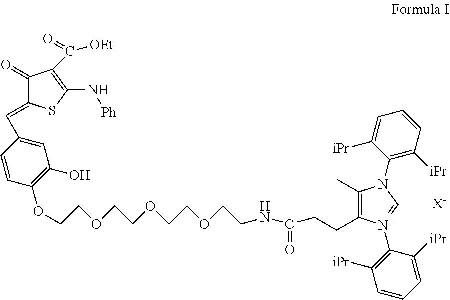

3. A method for cleaving target miRNA comprising contacting a mixture comprising at least an miRNA and RNase with a compound of Formula I of claim 2 wherein the miRNA is pri-miR-155 or pre-miR-155 or a combination thereof.

4. A method according to claim 3 wherein the miRNA is pre-miR-155.

5. A method according to claim 3 wherein the miRNA is a combination of pri-miR-155 and pre-miR-155.

6. A method according to claim 3 wherein the mixture is present in a cell and the mixture comprises a combination of pri-miR-155, pre-miR-155 and the cell further comprises the mature biogenesis product, miR-155.

7. A method according to claim 6 wherein the cell is an MDA-MD-231 cell.

8. A method according to claim 6 wherein the cell is a HUVEC.

9. A method according to claim 6 wherein of Formula I exhibits an IC so against pre-miR-155 at a concentration of no more than about 0.1 micromolar.

10. A method according to claim 6 wherein a dose of the compound of Formula I ranging from 1picomolar to 100 nanomolar decreases the cellular concentration of miR-155 in a dose related manner by at least 40%, preferably at least 60% more preferably at least 80%.

11. A method according to claim 7 er-9 wherein the cell is a breast cancer cell line.

12. A method according to claim 10 wherein the migratory ability of MDA-MD-231 cells is at least 50% inhibited by a concentration of the compound of Formula I of at least at 0.1 nanomolar.

13. A method according to claim 8 wherein the cell is a HUVEC.

14. A method according to claim 13 wherein the downstream proteins that are the targets of miR-155, including VHL, are depressed in HUVECs.

15. A method according to claim 13 wherein the tubule branching of HUVECs is at least 29% inhibited by a concentration of the compound of Formula I of at least at 0.1 nanomolar.

16. A method according to claim 1 in which the cell is affected by a disease in which miR-155 is over-expressed, including cancer, neuroinflammation, and neurodegeneration, amongst others

17. A method according to claim 16 wherein the complex is present in cells of an animal host.

18. A method according to claim 17 wherein the animal host is a rodent.

19. A method according to claim 1 wherein the complex is present in human patient-derived cells.

20. A method according to claim 1 wherein the complex is present in a human patient.

21. A method according to claim 1 wherein the gegenion is acetate, trifluoroacetate, mesylate, benzoate, chloride, sulfate, nitrate or phosphate.

22. A compound of Formula I, II, III, IV or VI 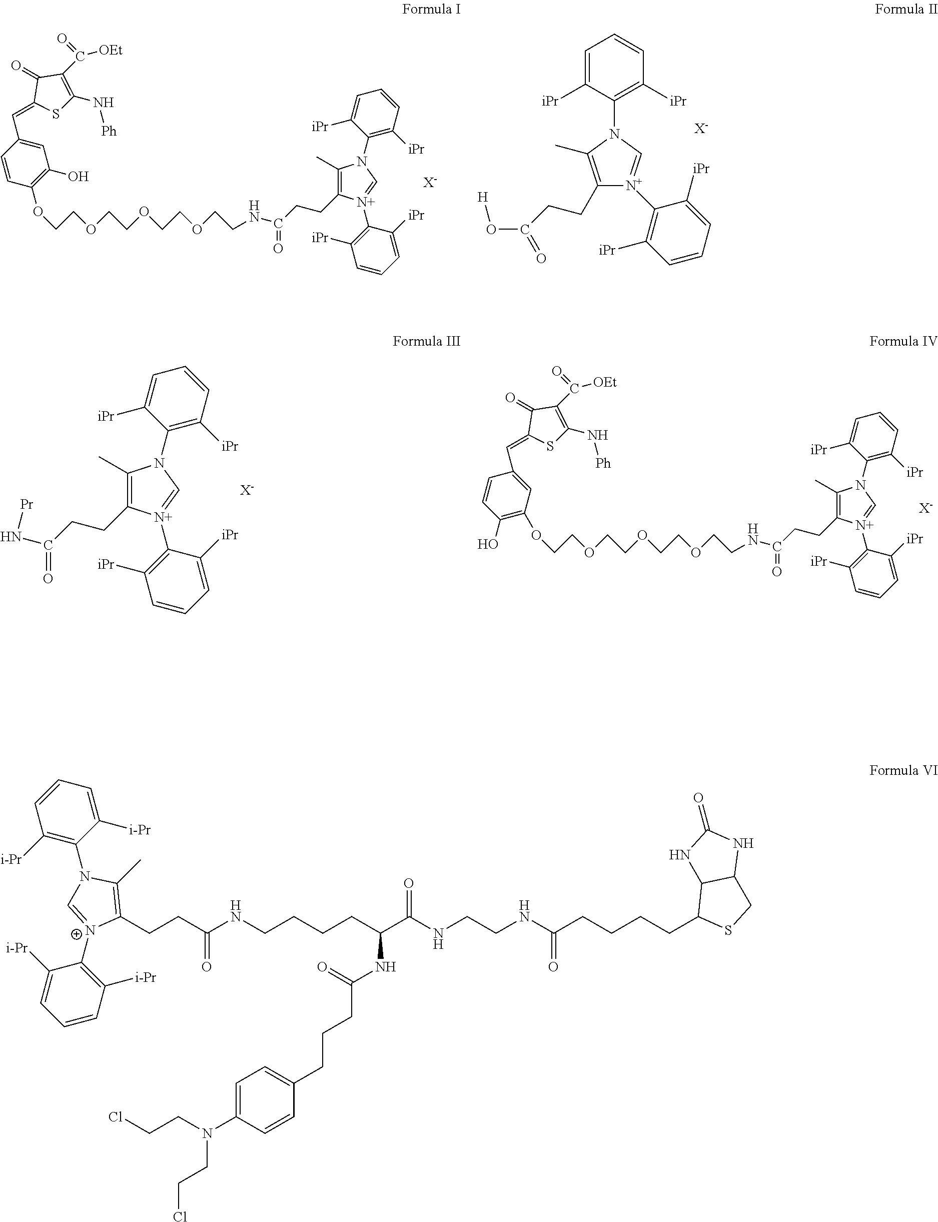
Wherein X is an organic or inorganic gegenion.

23. (canceled)

24. (canceled)

25. (canceled)

26. A compound according to claim 22, wherein the gegenion is acetate, trifluoroacetate, chloride, sulfate, nitrate or phosphate.

27. A pharmaceutical composition comprising a compound of Formula I of claim 22 in combination with a pharmaceutically acceptable carrier.
